SEQUENCE LISTING (68 sequences)

SEQ ID NO: 1 - DNA, 15 bp, Artificial Sequence
  Synthetic oligonucleotide
gaagaagaagaagaa

SEQ ID NO: 2 - DNA, 15 bp, Artificial Sequence
  Synthetic oligonucleotide
ttcttcttcttcttc

SEQ ID NO: 3 - DNA, 15 bp, Artificial Sequence
  Synthetic oligonucleotide
gggatcccttcagag

SEQ ID NO: 4 - DNA, 15 bp, Artificial Sequence
  Synthetic oligonucleotide
tggctggtacgccgc

SEQ ID NO: 5 - DNA, 15 bp, Artificial Sequence
  Synthetic oligonucleotide
acgccgcatgtatta

SEQ ID NO: 6 - DNA, 15 bp, Artificial Sequence
  Synthetic oligonucleotide
agatgaaagaggcag

SEQ ID NO: 7 - DNA, 15 bp, Artificial Sequence
  Synthetic oligonucleotide
gccacgtccaagcca

SEQ ID NO: 8 - DNA, 15 bp, Artificial Sequence
  Synthetic oligonucleotide
tatttgtgttgctct

SEQ ID NO: 9 - DNA, 15 bp, Artificial Sequence
  Synthetic oligonucleotide
ccggagtttgtactt

SEQ ID NO: 10 - DNA, 15 bp, Artificial Sequence
  Synthetic oligonucleotide
taggcttgaacttcc

SEQ ID NO: 11 - DNA, 15 bp, Artificial Sequence
  Synthetic oligonucleotide
cacacgtgttatttg

SEQ ID NO: 12 - DNA, 15 bp, Artificial Sequence
  Synthetic oligonucleotide
gcccacattgtgttt

SEQ ID NO: 13 - DNA, 15 bp, Artificial Sequence
  Synthetic oligonucleotide
gaagaaactttggga

SEQ ID NO: 14 - DNA, 15 bp, Artificial Sequence
  Synthetic oligonucleotide
ttggttgccagtgct

SEQ ID NO: 15 - DNA, 15 bp, Artificial Sequence
  Synthetic oligonucleotide
taaaagttaggactt

SEQ ID NO: 16 - DNA, 15 bp, Artificial Sequence
  Synthetic oligonucleotide
agaaaatggatttcc

SEQ ID NO: 17 - DNA, 15 bp, Artificial Sequence
  Synthetic oligonucleotide
tggcaggacgcggtg

SEQ ID NO: 18 - DNA, 15 bp, Artificial Sequence
  Synthetic oligonucleotide
ttagatctcctctag

SEQ ID NO: 19 - DNA, 15 bp, Artificial Sequence
  Synthetic oligonucleotide
gaaagcagacattta

SEQ ID NO: 20 - DNA, 15 bp, Artificial Sequence
  Synthetic oligonucleotide
ttacttggcttctgt

SEQ ID NO: 21 - DNA, 15 bp, Artificial Sequence
  Synthetic oligonucleotide
cactatctgagctgc

SEQ ID NO: 22 - DNA, 15 bp, Artificial Sequence
  Synthetic oligonucleotide
cacgtattgggcttc

SEQ ID NO: 23 - DNA, 15 bp, Artificial Sequence
  Synthetic oligonucleotide
cacccctgcctgtgt

SEQ ID NO: 24 - DNA, 15 bp, Artificial Sequence
  Synthetic oligonucleotide
ggacagcatgggttg

SEQ ID NO: 25 - DNA, 15 bp, Artificial Sequence
  Synthetic oligonucleotide
gtcagcagagttgtg

SEQ ID NO: 26 - DNA, 15 bp, Artificial Sequence
  Synthetic oligonucleotide
tggatttcccagcat

SEQ ID NO: 27 - DNA, 15 bp, Artificial Sequence
  Synthetic oligonucleotide
taggcaagtgtggcc

SEQ ID NO: 28 - DNA, 15 bp, Artificial Sequence
  Synthetic oligonucleotide
tggccatgatggtcc

SEQ ID NO: 29 - DNA, 15 bp, Artificial Sequence
  Synthetic oligonucleotide
ccggagttcaagact

SEQ ID NO: 30 - DNA, 15 bp, Artificial Sequence
  Synthetic oligonucleotide
aacccagtatctact

SEQ ID NO: 31 - DNA, 15 bp, Artificial Sequence
  Synthetic oligonucleotide
gttagccgggcgtgg

SEQ ID NO: 32 - DNA, 15 bp, Artificial Sequence
  Synthetic oligonucleotide
tgtaatcccagctac

SEQ ID NO: 33 - DNA, 15 bp, Artificial Sequence
  Synthetic oligonucleotide
tccagaggctgcggc

SEQ ID NO: 34 - DNA, 15 bp, Artificial Sequence
  Synthetic oligonucleotide
gaagaagaagaagaa

SEQ ID NO: 35 - DNA, 15 bp, Artificial Sequence
  Synthetic oligonucleotide
gaagaagaagaagaa

SEQ ID NO: 36 - DNA, 15 bp, Artificial Sequence
  Synthetic oligonucleotide
ttcttcttcttcttc

SEQ ID NO: 37 - DNA, 15 bp, Artificial Sequence
  Synthetic oligonucleotide
ttcttcttcttcttc

SEQ ID NO: 38 - DNA, 15 bp, Artificial Sequence
  Synthetic oligonucleotide
cttcttcttcttctt

SEQ ID NO: 39 - DNA, 15 bp, Artificial Sequence
  Synthetic oligonucleotide
cttcttcttcttctt

SEQ ID NO: 40 - DNA, 8 bp, Artificial Sequence
  Synthetic oligonucleotide
gaagaaga

SEQ ID NO: 41 - DNA, 8 bp, Artificial Sequence
  Synthetic oligonucleotide
aagaagaa

SEQ ID NO: 42 - DNA, 8 bp, Artificial Sequence
  Synthetic oligonucleotide
agaagaag

SEQ ID NO: 43 - DNA, 8 bp, Artificial Sequence
  Synthetic oligonucleotide
ttcttctt

SEQ ID NO: 44 - DNA, 8 bp, Artificial Sequence
  Synthetic oligonucleotide
cttcttct

SEQ ID NO: 45 - DNA, 15 bp, Artificial Sequence
  Synthetic oligonucleotide
aagaagaagaagaag

SEQ ID NO: 46 - DNA, 15 bp, Artificial Sequence
  Synthetic oligonucleotide
agaagaagaagaaga

SEQ ID NO: 47 - DNA, 15 bp, Artificial Sequence
  Synthetic oligonucleotide
tcttcttcttcttct

SEQ ID NO: 48 - DNA, 15 bp, Artificial Sequence
  Synthetic oligonucleotide
gaagaagaagaagaa

SEQ ID NO: 49 - DNA, 15 bp, Artificial Sequence
  Synthetic oligonucleotide
ttcttcttcttcttc

SEQ ID NO: 50 - DNA, 8 bp, Artificial Sequence
  Synthetic oligonucleotide
gaagaaga

SEQ ID NO: 51 - DNA, 8 bp, Artificial Sequence
  Synthetic oligonucleotide
aagaagaa

SEQ ID NO: 52 - DNA, 8 bp, Artificial Sequence
  Synthetic oligonucleotide
agaagaag

SEQ ID NO: 53 - DNA, 8 bp, Artificial Sequence
  Synthetic oligonucleotide
ttcttctt

SEQ ID NO: 54 - DNA, 8 bp, Artificial Sequence
  Synthetic oligonucleotide
cttcttct

SEQ ID NO: 55 - DNA, 8 bp, Artificial Sequence
  Synthetic oligonucleotide
tcttcttc

SEQ ID NO: 56 - DNA, 15 bp, Artificial Sequence
  Synthetic oligonucleotide
gaagaagaagaagaa

SEQ ID NO: 57 - DNA, 15 bp, Artificial Sequence
  Synthetic oligonucleotide
ttcttcttcttcttc

SEQ ID NO: 58 - DNA, 15 bp, Artificial Sequence
  Synthetic oligonucleotide
aagaagaagaagaag

SEQ ID NO: 59 - DNA, 15 bp, Artificial Sequence
  Synthetic oligonucleotide
agaagaagaagaaga

SEQ ID NO: 60 - DNA, 15 bp, Artificial Sequence
  Synthetic oligonucleotide
tcttcttcttcttct

SEQ ID NO: 61 - DNA, 15 bp, Artificial Sequence
  Synthetic oligonucleotide
cttcttcttcttctt

SEQ ID NO: 62 - DNA, 15 bp, Artificial Sequence
  Synthetic oligonucleotide
gaagaagaagaagaa

SEQ ID NO: 63 - DNA, 10201 bp, Homo sapiens
aaaaaaaaaaagagagagagagggagttagaaggaagatgcatcatttttatgacctggacttggaagtcaccaagcagcacttctgcagtaccctgttggttggaatagttgtagcccaaacccgaattcgaagggaggagaatagataacatccctgggtgacaggaatgtcaaagtcccaaacagcatatgacatgtgacaaatattggtgtggccttctttggaagatccaatcttccataccaggcaaagggatggaagactaaggaacaacatgagggatagccagagagggaaaaagcatcacttgttctaggaactacaaatagcttgaagaagcaaagatgtctagatgcctcccaatatgcagagtggggtgtacagaagagagtggtaagggcgctgggagagctaaggtgggcaagagagcttcctctgtcatgctaagaaagttggaatttatcttgatggtggtgaaagcagagggctatggttagattcacatttgagatttagatttttagatttaaaatgatcaccctggtgacactggcttaactcacaattttgcccaaggcctatgctaccacagtgcttctgaaactttaaagcacattagaatcacctggaggtcttgttaaaccatggattgctgggccttgaaaccccagagattctgattcagtagatcgagaatagggcctgagaatttgtatttctaacaagtttccaggtgatgctgaggctgctggcccagcgaccacatttgataatcatagccctctgataaatcctatcaaaatatcctaatggcagagcaagggaattctggtgatatcctcccctacccataacctgacagctattaggatctgcctacttgaggctaaaagcaaccaagagaggaacagctacagtgtaccacagagtccctcaacatctttgcccacgccacggtgccccagcttcttaccaagtgtgcctgattcctcttgactacctccaaggaagtggagaaagacaagttcttgcgaagccttcgtcttctctgatatgctattctatgtctatttctttggccaaaaagatggggcaatgatatcaactttgcagggagctggagcatttgctagtgacctttctatgccagaacttgctaagcatgctagctaataatgatgtagcacagggtgcggtggctcacgcctgtaatctcagcactttgggcggccgaggcgggcggatcacctgaggtcaggagttcgagaccagcctggccaacatgatgaaaccccatctctactaaaaatacaaaaattagccaggcgtggtggtgggcacctgcaatcccagctactctggaggctgagacagaatctcttgaacccaggaggtggagattgcagtgagcagagatggcaccactgcattccagcctgggcaacaaagcaagactctgtctcaaataataataataataataactaatgatgcagctttctctctctgagtatataatgcagttctgatgatgtgaggaagggcctcactgttggtgtggcagagtctgagaccatggctggcaatgaaaacactaccctttgatgcctatgggctctccctttatggtttcaaggagggcttctcaatcttggcagaattttggactggatagttctttgttgcacaggtggggggctgtcctgcacatcacaggatgtttcatccctggcctctacctactagatgccagtagaacatacccaccccacagctgcctgttgtgacaatcaaaagcatctccagatactttgcagggggaaaatgatttctccaggcctggcatatacataacagtatttaagcagctgcctagaattaattaaacacagaaggatgtctctcatccagaatgccctggaccacctctttgataggcaatcagatcccacctcctccaccctatttttgaaggccctgtgccaacaccacttcttccatgaatacttccttgattcccccatccctagctctatataaatctcccactcaacactcacacctgttagtttacattcctcttgacacttgtcatttagcatcctaagtatgtaaacatgtctctcttcacgattcacaaagtggctttggaagaactttagtaccttcccatcttctctgccatggaaagtgtacacaactgacattttctttttttttaagacagtatcttgctatgatggccgggctggaatgctgtggctattcacaggcacaatcatagctcactgcagccttgagctcccaggctcaagtgatcctcccgcctcagcctcctgagtagctgagatcacaggcatgcactaccacactcggctcacatttgacatcctctaaagcatatataaaatgtgaagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctgggcaacatagcgaaaccccctttctacaaaaaatacaaaaactagctgggtgtggtggtgcacacctgtagtcccagctacttggaaggctgaaatgggaagactgcttgagcccgggagggagaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgcagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcagtaagtatccgcgccgggaacagccgcgggccgcacgccgcgggccgcacgccgcacgcctgcgcagggaggcgccgcgcacgccggggtcgctccgggtacgcgcgctggactagctcaccccgctccttctcagggcggcccggcggaagcggccttgcaactcccttctctggttctcccggttgcatttacactggcttctgctttccgaaggaaaaggggacattttgtcctgcggtgcgactgcgggtcaaggcacgggcgaaggcagggcaggctggtggaggggaccggttccgaggggtgtgcggctgtctccatgcttgtcacttctctgcgataacttgtttcagtaatattaatagatggtatctgctagtatatacatacacataatgtgtgtgtctgtgtgtatctgtatatagcgtgtgtgttgtgtgtgtgtgtttgcgcgcacgggcgcgcgcacacctaatattttcaaggctggatttttttgaacgaaatgctttcctggaacgaggtgaaactttcagagctgcagaatagctagagcagcaggggccctggcttttggaaactgacccgacctttattccagattctgccccactccgcagagctgtgtgaccttgggggattcccctaacctctctgagacgtggctttgttttctgtagggagaagataaaggtgacgcccattttgcggacctggtgtgaggattaaatgggaataacatagataaagtcttcagaacttcaaattagttcccctttcttcctttggggggtacaaagaaatatctgacccagttacgccacggcttgaaaggaggaaacccaaagaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaagaagaagaagaagaagaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcccaaagtgctggaattacaggcgtgagccaccgcgtctggccatcagcagagtttttaatttaggagaatgacaagaggtggtacagttttttagatggtacctggtggctgttaagggctattgactgacaaacacacccaacttggcgctgccgcccaggaggtggacactgggtttctggatagatggttagcaacctctgtcaccagctgggcctctttttttctatactgaattaatcacatttgtttaacctgtctgttccatagttcccttgcacatcttgggtatttgaggagttgggtgggtggcagtggcaactggggccaccatcctgtttaattattttaaagccctgactgtcctggattgaccctaagctccccctggtctccaaaattcatcagaaactgagttcacttgaaggcctcttccccacccttttctccaccccttgcatctacttctaaagcagctgttcaacagaaacagaatgggagccacacacataattctacattttctagttaaaaagaaaaaaaaatcattttcaacaatatatttattcaacctagtacatacaaaatattatcattccaacatgtaatcagtattttaaaaatcagtaatgagaccaggcacggtggctcacgactgtaatcccaggactttgggaggccgaggcgagtggatcatctgagatcaggagttcaagaccagcctggccaacatggtgaaaccccatctctactaaaaactagctcagcatggtggtgggtgcctgtagtcccagctactcgggaggctgaggcatgagaatcacttgagcccaggaggcagaggttgcagtgagccaagattttgggggattctgtgacatacaaaaaaaatcagtaataagatatcttgcatactcttttcgtactcatatacttccagcatatctcaattcacaatttctaagtaaatgctctatctgtatttacttttataaaattcacaattaaaaatgaaggttcacatagtcaagttgttccaaacacacttaaatgtctcctaggctgggtgtggttgctcacacctgtaatcccagcactttgggaggctgagatgggcggatcacctgaggtcaggagtttgagaccagcctggccaacatggtgaaaccccgtctctactaaaaatacaaaaattagctggatgtggtggcactcacctgtaatcccagctactcaggaggctgaggcaggataattgcttgaacccgggaggtggtggaggttgcagtgagccgagatcgcaccactgccttccaacctgggcgacagagcgagactccgtctcaaaaaaaaaaaaaaggctcctaataactttattactttattatcacctcaaataattaaaattaaatgaagttgaaaatccaggtcctcagtcccattagccacatttctagtgctcagtagccacgggggctggtgaccaccacatgggacagcatatttagtacctgatcattggttctcagatctggctactcagcagaaccaagaatccacagaaacggcttttaaaagcacagccccacagcccccagccccagccttacctacctggaggctgggaaggactctgattccacgaggcagcctatgttttttgatggagggatgtgacaggggctgcatctttaacgtttcctcttaaatactggagacagcttcgaggaggagataactggatgtgtcttagtccatttgatggagggatgtgacggggctgcgtctttaacgtttcctcttaaataccggagacagcttcgagaaggagataactggatgtttcttagtccattttctgttgcttgtgacagaatacctgaaactgggcaatttatatggtaaaaaattttcttcttactgctctggaggctgagaagtccaaagtcaagtcccttcttgctggtggggactttgcagagtattgaggcggcaccgggcgtcatatggtaaggggctgagtgtgctacctcaggtgtctttttcttttcttataaagcctaactagtttcactcccatgataacccattaatctatgaatggattaatccattattgagggaagaaccttcatgacccagtcaccgcttaaaggccccacctctcaatactgccacatcgggaattaagtttcaacatgagtttcggaggtgacaaacattcaaaccatagcatgctgtctcttaaatgactcaataagctcctgtggcatccacttctgcatgccttgggcagcttttagacatctgtccattttcctagagggacaagaccaccacctgtgatcctatgaccttttggctttaggcctaacaagcaggttataccctcactcactttcaaatcatttttattgtcttgcagacaatttacacaagtttacacatagaaaaggatatgtaaatatttatacgctgccgggcgcggtggctcacgcctgtaatcccagcactttgggaggccgaggcaggtggatcacgagttcaggagatggagaccatcctggctaatacgatgaaaccccatctctactaaaaatacaaaaaattagccgggcgtggtgacgggtgcctgtagtccccactactcgggacgctgaggcaggagaatggcgtgaacccgggaggcagagcttgcagtgatccgagatcgtgccactgcactccagcctgggtgacagagcgagactgcatctcaaagaaaaaaataaataaataaataaatatttatactgcttataaactaataataaatgctatggtctgcatgtttgtgtcaccccaccattcatatgttaaaacctaatcaccaaagtgatattaggaggtggggcccttgggaggtgatgaggtatgagggtggagcccatatgattgggattagtgcccttctaaaatagcccaacggagcccagtgacaaggcatcatctatgaaccaggaaactggccctcaccagacaccaaagctgttggtgcattgatcttggatttcccaccctccaggactctaagaaacacatttctattgtttataagccacccagtggctggtattttgttataacatcccagactaagacaaataacaaatacttgtatccctgacaccaggttaagagatagaatttgtttgttcctctggaggcccttgtcttcaccccatcactgccctgtcctccctggaggaatctgccagcccgaattctgttcatcgtaccctccttttcttagagtttgacctcctctgtatctcccccaatccatgtattgcttatatacaaggtattctgctgtatctgttctgctatggcttgccccttttgttcaacactgtttttgtgcgtcatctgcattgatgcatgcagttgtcctttatttgttctcactgctggatagtatctggttgggtaaatatatcacactgtaaatcacactatccaggttcctttaggtgacatttggttgattgcagtgttctgttgttacgatggtgctgctgtgactgttcttgtgcatggacagaagttcctttcaggtgaatttctcagaatggaattgctgggcaaaggggcagccaataatcaactcatttgatgccaaaagtggtggtgccagttcatcctcccctgcgaggtatgggtcctgattcactcttcaagtgctgtggtttgacagggccgggggtgacaaggggacacctgggaaggaaagctgggctccctgctggccatccaggccagtccttaccagggggtaggcaatgattgggtcaagtggttcctgaccactgggcctgagacttcaggcccagaaactatctaatatttcctcaaatgcatcccatgagcaggcactgtgtgagtgagcacacacatctgaagcctcaagctaggcaagcctaccatgacttgtggtccaagggctcacgggtgacctggagttagagggagacatggctgccaggtggctttagaaagaacactcatcatggccaggtgcggtggcttacgcctgtaatcccagcactttgggaggccaaggtgggtggatcatgaggtcaggagtgagaccagcctgaccaacatgctgaaacctgtctctcctaaaaacacaaaaattagctgggcatggaggtgcacgcctgtaatcccagctactcaggaggctgaggcaggagaatcacttgaacccgggaggcggaggttgcaataagcctagattgtgccactgcattccagcctgggcaacagagcaagactccgtctcagaaaaaaaaaaaaaaaggaagaacactcatcctatgaccttgacctccaagctttgcctccctcaagcagaacagaatggagcctcccttaggcagaggcggaagttt

SEQ ID NO: 64 - DNA, 10201 bp, Homo sapiens
aaacttccgcctctgcctaagggaggctccattctgttctgcttgagggaggcaaagcttggaggtcaaggtcataggatgagtgttcttcctttttttttttttttctgagacggagtcttgctctgttgcccaggctggaatgcagtggcacaatctaggcttattgcaacctccgcctcccgggttcaagtgattctcctgcctcagcctcctgagtagctgggattacaggcgtgcacctccatgcccagctaatttttgtgtttttaggagagacaggtttcagcatgttggtcaggctggtctcactcctgacctcatgatccacccaccttggcctcccaaagtgctgggattacaggcgtaagccaccgcacctggccatgatgagtgttctttctaaagccacctggcagccatgtctccctctaactccaggtcacccgtgagcccttggaccacaagtcatggtaggcttgcctagcttgaggcttcagatgtgtgtgctcactcacacagtgcctgctcatgggatgcatttgaggaaatattagatagtttctgggcctgaagtctcaggcccagtggtcaggaaccacttgacccaatcattgcctaccccctggtaaggactggcctggatggccagcagggagcccagctttccttcccaggtgtccccttgtcacccccggccctgtcaaaccacagcacttgaagagtgaatcaggacccatacctcgcaggggaggatgaactggcaccaccacttttggcatcaaatgagttgattattggctgcccctttgcccagcaattccattctgagaaattcacctgaaaggaacttctgtccatgcacaagaacagtcacagcagcaccatcgtaacaacagaacactgcaatcaaccaaatgtcacctaaaggaacctggatagtgtgatttacagtgtgatatatttacccaaccagatactatccagcagtgagaacaaataaaggacaactgcatgcatcaatgcagatgacgcacaaaaacagtgttgaacaaaaggggcaagccatagcagaacagatacagcagaataccttgtatataagcaatacatggattgggggagatacagaggaggtcaaactctaagaaaaggagggtacgatgaacagaattcgggctggcagattcctccagggaggacagggcagtgatggggtgaagacaagggcctccagaggaacaaacaaattctatctcttaacctggtgtcagggatacaagtatttgttatttgtcttagtctgggatgttataacaaaataccagccactgggtggcttataaacaatagaaatgtgtttcttagagtcctggagggtgggaaatccaagatcaatgcaccaacagctttggtgtctggtgagggccagtttcctggttcatagatgatgccttgtcactgggctccgttgggctattttagaagggcactaatcccaatcatatgggctccaccctcatacctcatcacctcccaagggccccacctcctaatatcactttggtgattaggttttaacatatgaatggtggggtgacacaaacatgcagaccatagcatttattattagtttataagcagtataaatatttatttatttatttatttttttctttgagatgcagtctcgctctgtcacccaggctggagtgcagtggcacgatctcggatcactgcaagctctgcctcccgggttcacgccattctcctgcctcagcgtcccgagtagtggggactacaggcacccgtcaccacgcccggctaattttttgtatttttagtagagatggggtttcatcgtattagccaggatggtctccatctcctgaactcgtgatccacctgcctcggcctcccaaagtgctgggattacaggcgtgagccaccgcgcccggcagcgtataaatatttacatatccttttctatgtgtaaacttgtgtaaattgtctgcaagacaataaaaatgatttgaaagtgagtgagggtataacctgcttgttaggcctaaagccaaaaggtcataggatcacaggtggtggtcttgtccctctaggaaaatggacagatgtctaaaagctgcccaaggcatgcagaagtggatgccacaggagcttattgagtcatttaagagacagcatgctatggtttgaatgtttgtcacctccgaaactcatgttgaaacttaattcccgatgtggcagtattgagaggtggggcctttaagcggtgactgggtcatgaaggttcttccctcaataatggattaatccattcatagattaatgggttatcatgggagtgaaactagttaggctttataagaaaagaaaaagacacctgaggtagcacactcagccccttaccatatgacgcccggtgccgcctcaatactctgcaaagtccccaccagcaagaagggacttgactttggacttctcagcctccagagcagtaagaagaaaattttttaccatataaattgcccagtttcaggtattctgtcacaagcaacagaaaatggactaagaaacatccagttatctccttctcgaagctgtctccggtatttaagaggaaacgttaaagacgcagccccgtcacatccctccatcaaatggactaagacacatccagttatctcctcctcgaagctgtctccagtatttaagaggaaacgttaaagatgcagcccctgtcacatccctccatcaaaaaacataggctgcctcgtggaatcagagtccttcccagcctccaggtaggtaaggctggggctgggggctgtggggctgtgcttttaaaagccgtttctgtggattcttggttctgctgagtagccagatctgagaaccaatgatcaggtactaaatatgctgtcccatgtggtggtcaccagcccccgtggctactgagcactagaaatgtggctaatgggactgaggacctggattttcaacttcatttaattttaattatttgaggtgataataaagtaataaagttattaggagccttttttttttttttgagacggagtctcgctctgtcgcccaggttggaaggcagtggtgcgatctcggctcactgcaacctccaccacctcccgggttcaagcaattatcctgcctcagcctcctgagtagctgggattacaggtgagtgccaccacatccagctaatttttgtatttttagtagagacggggtttcaccatgttggccaggctggtctcaaactcctgacctcaggtgatccgcccatctcagcctcccaaagtgctgggattacaggtgtgagcaaccacacccagcctaggagacatttaagtgtgtttggaacaacttgactatgtgaaccttcatttttaattgtgaattttataaaagtaaatacagatagagcatttacttagaaattgtgaattgagatatgctggaagtatatgagtacgaaaagagtatgcaagatatcttattactgattttttttgtatgtcacagaatcccccaaaatcttggctcactgcaacctctgcctcctgggctcaagtgattctcatgcctcagcctcccgagtagctgggactacaggcacccaccaccatgctgagctagtttttagtagagatggggtttcaccatgttggccaggctggtcttgaactcctgatctcagatgatccactcgcctcggcctcccaaagtcctgggattacagtcgtgagccaccgtgcctggtctcattactgatttttaaaatactgattacatgttggaatgataatattttgtatgtactaggttgaataaatatattgttgaaaatgatttttttttctttttaactagaaaatgtagaattatgtgtgtggctcccattctgtttctgttgaacagctgctttagaagtagatgcaaggggtggagaaaagggtggggaagaggccttcaagtgaactcagtttctgatgaattttggagaccagggggagcttagggtcaatccaggacagtcagggctttaaaataattaaacaggatggtggccccagttgccactgccacccacccaactcctcaaatacccaagatgtgcaagggaactatggaacagacaggttaaacaaatgtgattaattcagtatagaaaaaaagaggcccagctggtgacagaggttgctaaccatctatccagaaacccagtgtccacctcctgggcggcagcgccaagttgggtgtgtttgtcagtcaatagcccttaacagccaccaggtaccatctaaaaaactgtaccacctcttgtcattctcctaaattaaaaactctgctgatggccagacgcggtggctcacgcctgtaattccagcactttgggagggcgaggtgggcggatcacctgaggttgggagttcgagaccagcctgaccaacatggagaaatctgtctctactaaaaatacaaaattagccgataatcccagctactcgggaggctgaggcaggagaatcacttgaacccaggaggcagaggttgcagtgagctgagactgagccactgcactccagcctgggcaacaagagggaaactctgtctcaaaaaaacaaaacaaaacacaactctgctgacaacccatgctgtccacacaggcaggggtggaagcccaatacgtggcagctcagatagtgcacagaagccaagtaataaatgtctgctttcctagaggagatctaaggaccatcatggccacacttgcctatttttccagagatgctgggaaatccattttattttttatttttatttattattattattttttgagacggagtcttgctctgtcgcccaggccggagtgcattgggcgatcttggcttaatgcaacctctgcctcccgggctcaagcgattctcctgccgcagcctctggagtagctgggattacaggcgcgcgacaccacgcccggctaacttttctttattttcttcttcttcttcttcttttttttttttttttgtattttttagtagatactgggtttcaccatgttggccaggttagtcttgaactccggacctcaggtgatccaccttcctaggcctcccaaagtgctgagattatgggcatgagccaccgcgtcctgccaggaaatccattttctaagtcctaacttttaagcactggcaaccaatcccaaagtttcttcaaacacaatgtgggccaaataacacgtgtgggaagttcaagcctaaagtacaaactccggagagcaacacaaatatggcttggacgtggcctgcctctttcatctcccctaatacatgcggcgtaccagccactctgaagggatccccttccgccttcctggcctttgcccagacggttccctcctcgtgaaacaccctctaccacttccttggcatcttcaagaccctcattaaataccatgtcctccccttgaggaatcttcctcatccccacagccattctttgggtttcctcctttcaagccgtggcgtaactgggtcagatatttctttgtaccccccaaaggaagaaaggggaactaatttgaagttctgaagactttatctatgttattcccatttaatcctcacaccaggtccgcaaaatgggcgtcacctttatcttctccctacagaaaacaaagccacgtctcagagaggttaggggaatcccccaaggtcacacagctctgcggagtggggcagaatctggaataaaggtcgggtcagtttccaaaagccagggcccctgctgctctagctattctgcagctctgaaagtttcacctcgttccaggaaagcatttcgttcaaaaaaatccagccttgaaaatattaggtgtgcgcgcgcccgtgcgcgcaaacacacacacacaacacacacgctatatacagatacacacagacacacacattatgtgtatgtatatactagcagataccatctattaatattactgaaacaagttatcgcagagaagtgacaagcatggagacagccgcacacccctcggaaccggtcccctccaccagcctgccctgccttcgcccgtgccttgacccgcagtcgcaccgcaggacaaaatgtccccttttccttcggaaagcagaagccagtgtaaatgcaaccgggagaaccagagaagggagttgcaaggccgcttccgccgggccgccctgagaaggagcggggtgagctagtccagcgcgcgtacccggagcgaccccggcgtgcgcggcgcctccctgcgcaggcgtgcggcgtgcggcccgcggcgtgcggcccgcggctgttcccggcgcggatacttactgcgcggcggggcgtgcaggtcgcatcgatgtcggtgcgcaggccacggcggccgcagagtggggccaactctgccggccgcgggacccgggtgagggtctgggcctgggctgggctgggtgacgccaggaggccggctactgcgcggcgcccgagagtccacatgctgctccgggtctgccgcccgctccgccctccagcgctgggtgctgcggcgacccctggtggccactggccgcaggcactcttctgtgggggagcagctagaggttagacctcaggaagaacttcccagcttagcactattcgtgcatttaacaaaaatggagagcctgctttgtgcaaagcacggagtgcaaccaggacccctgacccaagggagactgcagcctggtggcccgccgcttctaaaattctaaacaggaggaacttgggagctgctgtcttgctgggaagtgggtgcatgcacaaattgaggctgcttggccgccggtatgggtttacagcagttgggtatgtggggccaggagacggatgccttgtgtaaatctgtactctgcccacttcctagctgtgtgaccagagggcaagttaattaacctctctgtgccttgtttataaaatgcgttttataaaagttcctacttcataggattgtggtaaggataaaatgagttaagccctgtaaagttttttttggacagggcatgttcctgttaagtgcacaataaatattgttaatagattttattgattgattgattgattgagacggagtctcgctctgtcgcccaggctggagggcagtggtgcgatctcggctcactgcaagctctgcctcccggtttcacgccattctcctgcctcagcctcccgagtaactgggactacaggcgcccgccaccatgcctggctaattttttgtatttttttttttttttagtagagacgggtttcaccgtgttagccaggatggtctcgatctactgacctcgtgatccacccgcctcatttatttgtttgtttgagacagagtctcactctgttgcccaggctggagtgcagtggtgtggtcctggcttactgcaacttctccctcccgggctcaagcagtcttcccatttcagccttccaagtagctgggactacaggtgtgcaccaccacacccagctagtttttgtattttttgtagaaagggggtttcgctatgttgcccagggtggtcttgaacttctaggctcaaacgatctgcccgcctgggcctcacaaagtgctgagacgacaggcgtgagccaccgtgccttgcctattgttgatagattttaaaagagaattttatttctgttacatattacaaagggatgcaaattgtgaaagttttcttcacattttatatatgctttagaggatgtcaaatgtgagccgagtgtggtagtgcatgcctgtgatctcagctactcaggaggctgaggcgggaggatcacttgagcctgggagctcaaggctgcagtgagctatgattgtgcctgtgaatagccacagcattccagcccggccatcatagcaagatactgtcttaaaaaaaaagaaaatgtcagttgtgtacactttccatggcagagaagatgggaaggtactaaagttcttccaaagccactttgtgaatcgtgaagagagacatgtttacatacttaggatgctaaatgacaagtgtcaagaggaatgtaaactaacaggtgtgagtgttgagtgggagatttatatagagctagggatgggggaatcaaggaagtattcatggaagaagtggtgttggcacagggccttcaaaaatagggtggaggaggtgggatctgattgcctatcaaagaggtggtccagggcattctggatgagagacatccttctgtgtttaattaattctaggcagctgcttaaatactgttatgtatatgccaggcctggagaaatcattttccccctgcaaagtatctggagatgcttttgattgtcacaacaggcagctgtggggtgggtatgttctactggcatctagtaggtagaggccagggatgaaacatcctgtgatgtgcaggacagccccccacctgtgcaacaaagaactatccagtccaaaattctgccaagattgagaagccctccttgaaaccataaagggagagcccataggcatcaaagggtagtgttttcattgccagccatggtctcagactctgccacaccaacagtgaggcccttcctcacatcatcagaactgcattatatactcagagagagaaagctgcatcattagttattattattattattatttgagacagagtcttgctttgttgcccaggctggaatgcagtggtgccatctctgctcactgcaatctccacctcctgggttcaagagattctgtctcagcctccagagtagctgggattgcaggtgcccaccaccacgcctggctaatttttgtatttttagtagagatggggtttcatcatgttggccaggctggtctcgaactcctgacctcaggtgatccgcccgcctcggccgcccaaagtgctgagattacaggcgtgagccaccgcaccctgtgctacatcattattagctagcatgcttagcaagttctggcatagaaaggtcactagcaaatgctccagctccctgcaaagttgatatcattgccccatctttttggccaaagaaatagacatagaatagcatatcagagaagacgaaggcttcgcaagaacttgtctttctccacttccttggaggtagtcaagaggaatcaggcacacttggtaagaagctggggcaccgtggcgtgggcaaagatgttgagggactctgtggtacactgtagctgttcctctcttggttgcttttagcctcaagtaggcagatcctaatagctgtcaggttatgggtaggggaggatatcaccagaattcccttgctctgccattaggatattttgataggatttatcagagggctatgattatcaaatgtggtcgctgggccagcagcctcagcatcacctggaaacttgttagaaatacaaattctcaggccctattctcgatctactgaatcagaatctctggggtttcaaggcccagcaatccatggtttaacaagacctccaggtgattctaatgtgctttaaagtttcagaagcactgtggtagcataggccttgggcaaaattgtgagttaagccagtgtcaccagggtgatcattttaaatctaaaaatctaaatctcaaatgtgaatctaaccatagccctctgctttcaccaccatcaagataaattccaactttcttagcatgacagaggaagctctcttgcccaccttagctctcccagcgcccttaccactctcttctgtacaccccactctgcatattgggaggcatctagacatctttgcttcttcaagctatttgtagttcctagaacaagtgatgctttttccctctctggctatccctcatgttgttccttagtcttccatccctttgcctggtatggaagattggatcttccaaagaaggccacaccaatatttgtcacatgtcatatgctgtttgggactttgacattcctgtcacccagggatgttatctattctcctcccttcgaattcgggtttgggctacaactattccaaccaacagggtactgcagaagtgctgcttggtgacttccaagtccaggtcataaaaatgatgcatcttccttctaactccctctctctctcttttttttttt

SEQ ID NO: 65 - DNA, 4905 bp, Homo sapiens
aaaaaaaaaaagagagagagagggagttagaaggaagatgcatcatttttatgacctggacttggaagtcaccaagcagcacttctgcagtaccctgttggttggaatagttgtagcccaaacccgaattcgaagggaggagaatagataacatccctgggtgacaggaatgtcaaagtcccaaacagcatatgacatgtgacaaatattggtgtggccttctttggaagatccaatcttccataccaggcaaagggatggaagactaaggaacaacatgagggatagccagagagggaaaaagcatcacttgttctaggaactacaaatagcttgaagaagcaaagatgtctagatgcctcccaatatgcagagtggggtgtacagaagagagtggtaagggcgctgggagagctaaggtgggcaagagagcttcctctgtcatgctaagaaagttggaatttatcttgatggtggtgaaagcagagggctatggttagattcacatttgagatttagatttttagatttaaaatgatcaccctggtgacactggcttaactcacaattttgcccaaggcctatgctaccacagtgcttctgaaactttaaagcacattagaatcacctggaggtcttgttaaaccatggattgctgggccttgaaaccccagagattctgattcagtagatcgagaatagggcctgagaatttgtatttctaacaagtttccaggtgatgctgaggctgctggcccagcgaccacatttgataatcatagccctctgataaatcctatcaaaatatcctaatggcagagcaagggaattctggtgatatcctcccctacccataacctgacagctattaggatctgcctacttgaggctaaaagcaaccaagagaggaacagctacagtgtaccacagagtccctcaacatctttgcccacgccacggtgccccagcttcttaccaagtgtgcctgattcctcttgactacctccaaggaagtggagaaagacaagttcttgcgaagccttcgtcttctctgatatgctattctatgtctatttctttggccaaaaagatggggcaatgatatcaactttgcagggagctggagcatttgctagtgacctttctatgccagaacttgctaagcatgctagctaataatgatgtagcacagggtgcggtggctcacgcctgtaatctcagcactttgggcggccgaggcgggcggatcacctgaggtcaggagttcgagaccagcctggccaacatgatgaaaccccatctctactaaaaatacaaaaattagccaggcgtggtggtgggcacctgcaatcccagctactctggaggctgagacagaatctcttgaacccaggaggtggagattgcagtgagcagagatggcaccactgcattccagcctgggcaacaaagcaagactctgtctcaaataataataataataataactaatgatgcagctttctctctctgagtatataatgcagttctgatgatgtgaggaagggcctcactgttggtgtggcagagtctgagaccatggctggcaatgaaaacactaccctttgatgcctatgggctctccctttatggtttcaaggagggcttctcaatcttggcagaattttggactggatagttctttgttgcacaggtggggggctgtcctgcacatcacaggatgtttcatccctggcctctacctactagatgccagtagaacatacccaccccacagctgcctgttgtgacaatcaaaagcatctccagatactttgcagggggaaaatgatttctccaggcctggcatatacataacagtatttaagcagctgcctagaattaattaaacacagaaggatgtctctcatccagaatgccctggaccacctctttgataggcaatcagatcccacctcctccaccctatttttgaaggccctgtgccaacaccacttcttccatgaatacttccttgattcccccatccctagctctatataaatctcccactcaacactcacacctgttagtttacattcctcttgacacttgtcatttagcatcctaagtatgtaaacatgtctctcttcacgattcacaaagtggctttggaagaactttagtaccttcccatcttctctgccatggaaagtgtacacaactgacattttctttttttttaagacagtatcttgctatgatggccgggctggaatgctgtggctattcacaggcacaatcatagctcactgcagccttgagctcccaggctcaagtgatcctcccgcctcagcctcctgagtagctgagatcacaggcatgcactaccacactcggctcacatttgacatcctctaaagcatatataaaatgtgaagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctgggcaacatagcgaaaccccctttctacaaaaaatacaaaaactagctgggtgtggtggtgcacacctgtagtcccagctacttggaaggctgaaatgggaagactgcttgagcccgggagggagaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgcagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcagtaagtatccgcgccgggaacagccgcgggccgcacgccgcgggccgcacgccgcacgcctgcgcagggaggcgccgcgcacgccggggtcgctccgggtacgcgcgctggactagctcaccccgctccttctcagggcggcccggcggaagcggccttgcaactcccttctctggttctcccggttgcatttacactggcttctgctttccgaaggaaaaggggacattttgtcctgcggtgcgactgcgggtcaaggcacgggcgaaggcagggcaggctggtggaggggaccggttccgaggggtgtgcggctgtctccatgcttgtcacttctctgcgataacttgtttcagtaatattaatagatggtatctgctagtatatacatacacataatgtgtgtgtctgtgtgtatctgtatatagcgtgtgtgttgtgtgtgtgtgtttgcgcgcacgggcgcgcgcacacctaatattttcaaggctggatttttttgaacgaaatgctttcctggaacgaggtgaaactttcagagctgcagaatagctagagcagcaggggccctggcttttggaaactgacccgacctttattccagattctgccccactccgcagagctgtgtgaccttgggggattcccctaacctctctgagacgtggctttgttttctgtagggagaagataaaggtgacgcccattttgcggacctggtgtgaggattaaatgggaataacatagataaagtcttcagaacttcaaattagttcccctttcttcctttggggggtacaaagaaatatctgacccagttacgccacggcttgaaaggaggaaacccaaagaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcc

SEQ ID NO: 66 - DNA, 4905 bp, Homo sapiens
ggaagttcaagcctaaagtacaaactccggagagcaacacaaatatggcttggacgtggcctgcctctttcatctcccctaatacatgcggcgtaccagccactctgaagggatccccttccgccttcctggcctttgcccagacggttccctcctcgtgaaacaccctctaccacttccttggcatcttcaagaccctcattaaataccatgtcctccccttgaggaatcttcctcatccccacagccattctttgggtttcctcctttcaagccgtggcgtaactgggtcagatatttctttgtaccccccaaaggaagaaaggggaactaatttgaagttctgaagactttatctatgttattcccatttaatcctcacaccaggtccgcaaaatgggcgtcacctttatcttctccctacagaaaacaaagccacgtctcagagaggttaggggaatcccccaaggtcacacagctctgcggagtggggcagaatctggaataaaggtcgggtcagtttccaaaagccagggcccctgctgctctagctattctgcagctctgaaagtttcacctcgttccaggaaagcatttcgttcaaaaaaatccagccttgaaaatattaggtgtgcgcgcgcccgtgcgcgcaaacacacacacacaacacacacgctatatacagatacacacagacacacacattatgtgtatgtatatactagcagataccatctattaatattactgaaacaagttatcgcagagaagtgacaagcatggagacagccgcacacccctcggaaccggtcccctccaccagcctgccctgccttcgcccgtgccttgacccgcagtcgcaccgcaggacaaaatgtccccttttccttcggaaagcagaagccagtgtaaatgcaaccgggagaaccagagaagggagttgcaaggccgcttccgccgggccgccctgagaaggagcggggtgagctagtccagcgcgcgtacccggagcgaccccggcgtgcgcggcgcctccctgcgcaggcgtgcggcgtgcggcccgcggcgtgcggcccgcggctgttcccggcgcggatacttactgcgcggcggggcgtgcaggtcgcatcgatgtcggtgcgcaggccacggcggccgcagagtggggccaactctgccggccgcgggacccgggtgagggtctgggcctgggctgggctgggtgacgccaggaggccggctactgcgcggcgcccgagagtccacatgctgctccgggtctgccgcccgctccgccctccagcgctgggtgctgcggcgacccctggtggccactggccgcaggcactcttctgtgggggagcagctagaggttagacctcaggaagaacttcccagcttagcactattcgtgcatttaacaaaaatggagagcctgctttgtgcaaagcacggagtgcaaccaggacccctgacccaagggagactgcagcctggtggcccgccgcttctaaaattctaaacaggaggaacttgggagctgctgtcttgctgggaagtgggtgcatgcacaaattgaggctgcttggccgccggtatgggtttacagcagttgggtatgtggggccaggagacggatgccttgtgtaaatctgtactctgcccacttcctagctgtgtgaccagagggcaagttaattaacctctctgtgccttgtttataaaatgcgttttataaaagttcctacttcataggattgtggtaaggataaaatgagttaagccctgtaaagttttttttggacagggcatgttcctgttaagtgcacaataaatattgttaatagattttattgattgattgattgattgagacggagtctcgctctgtcgcccaggctggagggcagtggtgcgatctcggctcactgcaagctctgcctcccggtttcacgccattctcctgcctcagcctcccgagtaactgggactacaggcgcccgccaccatgcctggctaattttttgtatttttttttttttttagtagagacgggtttcaccgtgttagccaggatggtctcgatctactgacctcgtgatccacccgcctcatttatttgtttgtttgagacagagtctcactctgttgcccaggctggagtgcagtggtgtggtcctggcttactgcaacttctccctcccgggctcaagcagtcttcccatttcagccttccaagtagctgggactacaggtgtgcaccaccacacccagctagtttttgtattttttgtagaaagggggtttcgctatgttgcccagggtggtcttgaacttctaggctcaaacgatctgcccgcctgggcctcacaaagtgctgagacgacaggcgtgagccaccgtgccttgcctattgttgatagattttaaaagagaattttatttctgttacatattacaaagggatgcaaattgtgaaagttttcttcacattttatatatgctttagaggatgtcaaatgtgagccgagtgtggtagtgcatgcctgtgatctcagctactcaggaggctgaggcgggaggatcacttgagcctgggagctcaaggctgcagtgagctatgattgtgcctgtgaatagccacagcattccagcccggccatcatagcaagatactgtcttaaaaaaaaagaaaatgtcagttgtgtacactttccatggcagagaagatgggaaggtactaaagttcttccaaagccactttgtgaatcgtgaagagagacatgtttacatacttaggatgctaaatgacaagtgtcaagaggaatgtaaactaacaggtgtgagtgttgagtgggagatttatatagagctagggatgggggaatcaaggaagtattcatggaagaagtggtgttggcacagggccttcaaaaatagggtggaggaggtgggatctgattgcctatcaaagaggtggtccagggcattctggatgagagacatccttctgtgtttaattaattctaggcagctgcttaaatactgttatgtatatgccaggcctggagaaatcattttccccctgcaaagtatctggagatgcttttgattgtcacaacaggcagctgtggggtgggtatgttctactggcatctagtaggtagaggccagggatgaaacatcctgtgatgtgcaggacagccccccacctgtgcaacaaagaactatccagtccaaaattctgccaagattgagaagccctccttgaaaccataaagggagagcccataggcatcaaagggtagtgttttcattgccagccatggtctcagactctgccacaccaacagtgaggcccttcctcacatcatcagaactgcattatatactcagagagagaaagctgcatcattagttattattattattattatttgagacagagtcttgctttgttgcccaggctggaatgcagtggtgccatctctgctcactgcaatctccacctcctgggttcaagagattctgtctcagcctccagagtagctgggattgcaggtgcccaccaccacgcctggctaatttttgtatttttagtagagatggggtttcatcatgttggccaggctggtctcgaactcctgacctcaggtgatccgcccgcctcggccgcccaaagtgctgagattacaggcgtgagccaccgcaccctgtgctacatcattattagctagcatgcttagcaagttctggcatagaaaggtcactagcaaatgctccagctccctgcaaagttgatatcattgccccatctttttggccaaagaaatagacatagaatagcatatcagagaagacgaaggcttcgcaagaacttgtctttctccacttccttggaggtagtcaagaggaatcaggcacacttggtaagaagctggggcaccgtggcgtgggcaaagatgttgagggactctgtggtacactgtagctgttcctctcttggttgcttttagcctcaagtaggcagatcctaatagctgtcaggttatgggtaggggaggatatcaccagaattcccttgctctgccattaggatattttgataggatttatcagagggctatgattatcaaatgtggtcgctgggccagcagcctcagcatcacctggaaacttgttagaaatacaaattctcaggccctattctcgatctactgaatcagaatctctggggtttcaaggcccagcaatccatggtttaacaagacctccaggtgattctaatgtgctttaaagtttcagaagcactgtggtagcataggccttgggcaaaattgtgagttaagccagtgtcaccagggtgatcattttaaatctaaaaatctaaatctcaaatgtgaatctaaccatagccctctgctttcaccaccatcaagataaattccaactttcttagcatgacagaggaagctctcttgcccaccttagctctcccagcgcccttaccactctcttctgtacaccccactctgcatattgggaggcatctagacatctttgcttcttcaagctatttgtagttcctagaacaagtgatgctttttccctctctggctatccctcatgttgttccttagtcttccatccctttgcctggtatggaagattggatcttccaaagaaggccacaccaatatttgtcacatgtcatatgctgtttgggactttgacattcctgtcacccagggatgttatctattctcctcccttcgaattcgggtttgggctacaactattccaaccaacagggtactgcagaagtgctgcttggtgacttccaagtccaggtcataaaaatgatgcatcttccttctaactccctctctctctcttttttttttt

SEQ ID NO: 67 - DNA, 4795 bp, Homo sapiens
cttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcccaaagtgctggaattacaggcgtgagccaccgcgtctggccatcagcagagtttttaatttaggagaatgacaagaggtggtacagttttttagatggtacctggtggctgttaagggctattgactgacaaacacacccaacttggcgctgccgcccaggaggtggacactgggtttctggatagatggttagcaacctctgtcaccagctgggcctctttttttctatactgaattaatcacatttgtttaacctgtctgttccatagttcccttgcacatcttgggtatttgaggagttgggtgggtggcagtggcaactggggccaccatcctgtttaattattttaaagccctgactgtcctggattgaccctaagctccccctggtctccaaaattcatcagaaactgagttcacttgaaggcctcttccccacccttttctccaccccttgcatctacttctaaagcagctgttcaacagaaacagaatgggagccacacacataattctacattttctagttaaaaagaaaaaaaaatcattttcaacaatatatttattcaacctagtacatacaaaatattatcattccaacatgtaatcagtattttaaaaatcagtaatgagaccaggcacggtggctcacgactgtaatcccaggactttgggaggccgaggcgagtggatcatctgagatcaggagttcaagaccagcctggccaacatggtgaaaccccatctctactaaaaactagctcagcatggtggtgggtgcctgtagtcccagctactcgggaggctgaggcatgagaatcacttgagcccaggaggcagaggttgcagtgagccaagattttgggggattctgtgacatacaaaaaaaatcagtaataagatatcttgcatactcttttcgtactcatatacttccagcatatctcaattcacaatttctaagtaaatgctctatctgtatttacttttataaaattcacaattaaaaatgaaggttcacatagtcaagttgttccaaacacacttaaatgtctcctaggctgggtgtggttgctcacacctgtaatcccagcactttgggaggctgagatgggcggatcacctgaggtcaggagtttgagaccagcctggccaacatggtgaaaccccgtctctactaaaaatacaaaaattagctggatgtggtggcactcacctgtaatcccagctactcaggaggctgaggcaggataattgcttgaacccgggaggtggtggaggttgcagtgagccgagatcgcaccactgccttccaacctgggcgacagagcgagactccgtctcaaaaaaaaaaaaaaggctcctaataactttattactttattatcacctcaaataattaaaattaaatgaagttgaaaatccaggtcctcagtcccattagccacatttctagtgctcagtagccacgggggctggtgaccaccacatgggacagcatatttagtacctgatcattggttctcagatctggctactcagcagaaccaagaatccacagaaacggcttttaaaagcacagccccacagcccccagccccagccttacctacctggaggctgggaaggactctgattccacgaggcagcctatgttttttgatggagggatgtgacaggggctgcatctttaacgtttcctcttaaatactggagacagcttcgaggaggagataactggatgtgtcttagtccatttgatggagggatgtgacggggctgcgtctttaacgtttcctcttaaataccggagacagcttcgagaaggagataactggatgtttcttagtccattttctgttgcttgtgacagaatacctgaaactgggcaatttatatggtaaaaaattttcttcttactgctctggaggctgagaagtccaaagtcaagtcccttcttgctggtggggactttgcagagtattgaggcggcaccgggcgtcatatggtaaggggctgagtgtgctacctcaggtgtctttttcttttcttataaagcctaactagtttcactcccatgataacccattaatctatgaatggattaatccattattgagggaagaaccttcatgacccagtcaccgcttaaaggccccacctctcaatactgccacatcgggaattaagtttcaacatgagtttcggaggtgacaaacattcaaaccatagcatgctgtctcttaaatgactcaataagctcctgtggcatccacttctgcatgccttgggcagcttttagacatctgtccattttcctagagggacaagaccaccacctgtgatcctatgaccttttggctttaggcctaacaagcaggttataccctcactcactttcaaatcatttttattgtcttgcagacaatttacacaagtttacacatagaaaaggatatgtaaatatttatacgctgccgggcgcggtggctcacgcctgtaatcccagcactttgggaggccgaggcaggtggatcacgagttcaggagatggagaccatcctggctaatacgatgaaaccccatctctactaaaaatacaaaaaattagccgggcgtggtgacgggtgcctgtagtccccactactcgggacgctgaggcaggagaatggcgtgaacccgggaggcagagcttgcagtgatccgagatcgtgccactgcactccagcctgggtgacagagcgagactgcatctcaaagaaaaaaataaataaataaataaatatttatactgcttataaactaataataaatgctatggtctgcatgtttgtgtcaccccaccattcatatgttaaaacctaatcaccaaagtgatattaggaggtggggcccttgggaggtgatgaggtatgagggtggagcccatatgattgggattagtgcccttctaaaatagcccaacggagcccagtgacaaggcatcatctatgaaccaggaaactggccctcaccagacaccaaagctgttggtgcattgatcttggatttcccaccctccaggactctaagaaacacatttctattgtttataagccacccagtggctggtattttgttataacatcccagactaagacaaataacaaatacttgtatccctgacaccaggttaagagatagaatttgtttgttcctctggaggcccttgtcttcaccccatcactgccctgtcctccctggaggaatctgccagcccgaattctgttcatcgtaccctccttttcttagagtttgacctcctctgtatctcccccaatccatgtattgcttatatacaaggtattctgctgtatctgttctgctatggcttgccccttttgttcaacactgtttttgtgcgtcatctgcattgatgcatgcagttgtcctttatttgttctcactgctggatagtatctggttgggtaaatatatcacactgtaaatcacactatccaggttcctttaggtgacatttggttgattgcagtgttctgttgttacgatggtgctgctgtgactgttcttgtgcatggacagaagttcctttcaggtgaatttctcagaatggaattgctgggcaaaggggcagccaataatcaactcatttgatgccaaaagtggtggtgccagttcatcctcccctgcgaggtatgggtcctgattcactcttcaagtgctgtggtttgacagggccgggggtgacaaggggacacctgggaaggaaagctgggctccctgctggccatccaggccagtccttaccagggggtaggcaatgattgggtcaagtggttcctgaccactgggcctgagacttcaggcccagaaactatctaatatttcctcaaatgcatcccatgagcaggcactgtgtgagtgagcacacacatctgaagcctcaagctaggcaagcctaccatgacttgtggtccaagggctcacgggtgacctggagttagagggagacatggctgccaggtggctttagaaagaacactcatcatggccaggtgcggtggcttacgcctgtaatcccagcactttgggaggccaaggtgggtggatcatgaggtcaggagtgagaccagcctgaccaacatgctgaaacctgtctctcctaaaaacacaaaaattagctgggcatggaggtgcacgcctgtaatcccagctactcaggaggctgaggcaggagaatcacttgaacccgggaggcggaggttgcaataagcctagattgtgccactgcattccagcctgggcaacagagcaagactccgtctcagaaaaaaaaaaaaaaaggaagaacactcatcctatgaccttgacctccaagctttgcctccctcaagcagaacagaatggagcctcccttaggcagaggcggaagttt

SEQ ID NO: 68 - DNA, 4795 bp, Homo sapiens
aaacttccgcctctgcctaagggaggctccattctgttctgcttgagggaggcaaagcttggaggtcaaggtcataggatgagtgttcttcctttttttttttttttctgagacggagtcttgctctgttgcccaggctggaatgcagtggcacaatctaggcttattgcaacctccgcctcccgggttcaagtgattctcctgcctcagcctcctgagtagctgggattacaggcgtgcacctccatgcccagctaatttttgtgtttttaggagagacaggtttcagcatgttggtcaggctggtctcactcctgacctcatgatccacccaccttggcctcccaaagtgctgggattacaggcgtaagccaccgcacctggccatgatgagtgttctttctaaagccacctggcagccatgtctccctctaactccaggtcacccgtgagcccttggaccacaagtcatggtaggcttgcctagcttgaggcttcagatgtgtgtgctcactcacacagtgcctgctcatgggatgcatttgaggaaatattagatagtttctgggcctgaagtctcaggcccagtggtcaggaaccacttgacccaatcattgcctaccccctggtaaggactggcctggatggccagcagggagcccagctttccttcccaggtgtccccttgtcacccccggccctgtcaaaccacagcacttgaagagtgaatcaggacccatacctcgcaggggaggatgaactggcaccaccacttttggcatcaaatgagttgattattggctgcccctttgcccagcaattccattctgagaaattcacctgaaaggaacttctgtccatgcacaagaacagtcacagcagcaccatcgtaacaacagaacactgcaatcaaccaaatgtcacctaaaggaacctggatagtgtgatttacagtgtgatatatttacccaaccagatactatccagcagtgagaacaaataaaggacaactgcatgcatcaatgcagatgacgcacaaaaacagtgttgaacaaaaggggcaagccatagcagaacagatacagcagaataccttgtatataagcaatacatggattgggggagatacagaggaggtcaaactctaagaaaaggagggtacgatgaacagaattcgggctggcagattcctccagggaggacagggcagtgatggggtgaagacaagggcctccagaggaacaaacaaattctatctcttaacctggtgtcagggatacaagtatttgttatttgtcttagtctgggatgttataacaaaataccagccactgggtggcttataaacaatagaaatgtgtttcttagagtcctggagggtgggaaatccaagatcaatgcaccaacagctttggtgtctggtgagggccagtttcctggttcatagatgatgccttgtcactgggctccgttgggctattttagaagggcactaatcccaatcatatgggctccaccctcatacctcatcacctcccaagggccccacctcctaatatcactttggtgattaggttttaacatatgaatggtggggtgacacaaacatgcagaccatagcatttattattagtttataagcagtataaatatttatttatttatttatttttttctttgagatgcagtctcgctctgtcacccaggctggagtgcagtggcacgatctcggatcactgcaagctctgcctcccgggttcacgccattctcctgcctcagcgtcccgagtagtggggactacaggcacccgtcaccacgcccggctaattttttgtatttttagtagagatggggtttcatcgtattagccaggatggtctccatctcctgaactcgtgatccacctgcctcggcctcccaaagtgctgggattacaggcgtgagccaccgcgcccggcagcgtataaatatttacatatccttttctatgtgtaaacttgtgtaaattgtctgcaagacaataaaaatgatttgaaagtgagtgagggtataacctgcttgttaggcctaaagccaaaaggtcataggatcacaggtggtggtcttgtccctctaggaaaatggacagatgtctaaaagctgcccaaggcatgcagaagtggatgccacaggagcttattgagtcatttaagagacagcatgctatggtttgaatgtttgtcacctccgaaactcatgttgaaacttaattcccgatgtggcagtattgagaggtggggcctttaagcggtgactgggtcatgaaggttcttccctcaataatggattaatccattcatagattaatgggttatcatgggagtgaaactagttaggctttataagaaaagaaaaagacacctgaggtagcacactcagccccttaccatatgacgcccggtgccgcctcaatactctgcaaagtccccaccagcaagaagggacttgactttggacttctcagcctccagagcagtaagaagaaaattttttaccatataaattgcccagtttcaggtattctgtcacaagcaacagaaaatggactaagaaacatccagttatctccttctcgaagctgtctccggtatttaagaggaaacgttaaagacgcagccccgtcacatccctccatcaaatggactaagacacatccagttatctcctcctcgaagctgtctccagtatttaagaggaaacgttaaagatgcagcccctgtcacatccctccatcaaaaaacataggctgcctcgtggaatcagagtccttcccagcctccaggtaggtaaggctggggctgggggctgtggggctgtgcttttaaaagccgtttctgtggattcttggttctgctgagtagccagatctgagaaccaatgatcaggtactaaatatgctgtcccatgtggtggtcaccagcccccgtggctactgagcactagaaatgtggctaatgggactgaggacctggattttcaacttcatttaattttaattatttgaggtgataataaagtaataaagttattaggagccttttttttttttttgagacggagtctcgctctgtcgcccaggttggaaggcagtggtgcgatctcggctcactgcaacctccaccacctcccgggttcaagcaattatcctgcctcagcctcctgagtagctgggattacaggtgagtgccaccacatccagctaatttttgtatttttagtagagacggggtttcaccatgttggccaggctggtctcaaactcctgacctcaggtgatccgcccatctcagcctcccaaagtgctgggattacaggtgtgagcaaccacacccagcctaggagacatttaagtgtgtttggaacaacttgactatgtgaaccttcatttttaattgtgaattttataaaagtaaatacagatagagcatttacttagaaattgtgaattgagatatgctggaagtatatgagtacgaaaagagtatgcaagatatcttattactgattttttttgtatgtcacagaatcccccaaaatcttggctcactgcaacctctgcctcctgggctcaagtgattctcatgcctcagcctcccgagtagctgggactacaggcacccaccaccatgctgagctagtttttagtagagatggggtttcaccatgttggccaggctggtcttgaactcctgatctcagatgatccactcgcctcggcctcccaaagtcctgggattacagtcgtgagccaccgtgcctggtctcattactgatttttaaaatactgattacatgttggaatgataatattttgtatgtactaggttgaataaatatattgttgaaaatgatttttttttctttttaactagaaaatgtagaattatgtgtgtggctcccattctgtttctgttgaacagctgctttagaagtagatgcaaggggtggagaaaagggtggggaagaggccttcaagtgaactcagtttctgatgaattttggagaccagggggagcttagggtcaatccaggacagtcagggctttaaaataattaaacaggatggtggccccagttgccactgccacccacccaactcctcaaatacccaagatgtgcaagggaactatggaacagacaggttaaacaaatgtgattaattcagtatagaaaaaaagaggcccagctggtgacagaggttgctaaccatctatccagaaacccagtgtccacctcctgggcggcagcgccaagttgggtgtgtttgtcagtcaatagcccttaacagccaccaggtaccatctaaaaaactgtaccacctcttgtcattctcctaaattaaaaactctgctgatggccagacgcggtggctcacgcctgtaattccagcactttgggagggcgaggtgggcggatcacctgaggttgggagttcgagaccagcctgaccaacatggagaaatctgtctctactaaaaatacaaaattagccgataatcccagctactcgggaggctgaggcaggagaatcacttgaacccaggaggcagaggttgcagtgagctgagactgagccactgcactccagcctgggcaacaagagggaaactctgtctcaaaaaaacaaaacaaaacacaactctgctgacaacccatgctgtccacacaggcaggggtggaagcccaatacgtggcagctcagatagtgcacagaagccaagtaataaatgtctgctttcctagaggagatctaag

(19) United States
(12) Patent Application Publication
(10) Pub. No.: US 2015/0225722 A1
(43) Pub. Date: Aug. 13, 2015

(54) METHODS FOR SELECTIVE TARGETING OF HETEROCHROMATIN FORMING NON-CODING RNA

(71) Applicant: RaNA Therapeutics, Inc., Cambridge, MA (US)

(72) Inventor: Fatih Ozsolak, Boston, MA (US)

(73) Assignee: RaNA Therapeutics, Inc., Cambridge, MA (US)

(21) Appl. No.: 14/699,130

(22) Filed: Apr. 29, 2015

Related U.S. Application Data
(63) Continuation of application No. PCT/US2014/051257, filed on Aug. 15, 2014.
(60) Provisional application No. 61/866,894, filed on Aug. 16, 2013.

(51) Int. Cl. C12N 15/113

(52) U.S. Cl. CPC C12N 15/113; C12N 2310/113; C12N 2310/341; C12N 2310/3231; C12N 2310/321

(57) ABSTRACT

Provided herein are oligonucleotides that are useful for modulating the heterochromatin state of genes; related compositions and methods are also provided. In some embodiments, methods are provided for treating a disease associated with heterochromatin formation, including diseases associated with repeat expansion within genes.

### CROSS-REFERENCE TO RELATED APPLICATIONS

This application claims the benefit under 35 U.S.C. §119(e) of U.S. Provisional Application No. 61/866,894, entitled “HETEROCHROMATIN FORMING NON-CODING RNAS”, filed Aug. 16, 2013, the contents of which are incorporated herein by reference in its entirety.

### FIELD OF THE INVENTION

The invention relates in part to oligonucleotide based compositions, as well as methods of using oligonucleotide based compositions to modulate gene expression.

### BACKGROUND OF THE INVENTION

A considerable portion of human diseases can be treated by selectively altering protein and/or RNA levels of disease-associated transcription units. Such methods typically involve blocking translation of mRNAs or causing degradation of target RNAs. However, additional approaches for modulating gene expression are desirable, including methods for increasing expression levels as limited approaches.

### SUMMARY OF THE INVENTION

According to some aspects of the invention, compositions and methods are provided for increasing gene expression in a targeted and specific manner. In some embodiments, it has been discovered that oligonucleotides complementary to sequences in a genomic region encoding heterochromatin forming non-coding RNAs are useful for eliminating or reversing heterochromatin at genes regulated by the non-coding RNAs. Accordingly, in some embodiments, methods are provided for increasing expression of genes that have been downregulated or silenced due to heterochromatin formation. In some embodiments, methods are provided for treating a condition or disease associated with decreased levels of a gene due to heterochromatin formation. In some embodiments, the genes of interest contain repetitive sequences (e.g., triplet repeats) that are associated with the heterchromatin formation. Thus, in some embodiments, methods are provided for treating diseases or conditions associated with repetitive sequences (e.g., triplet repeat expansion genes). In some embodiments, oligonucleotides are provided that are complementary with a heterochromatin forming non-coding RNA or a reverse complement thereof and that have chemistries suitable for delivery, hybridization and stability within cells. In some embodiments, oligonucleotide chemistries are provided that are useful for controlling the pharmacokinetics, biodistribution, bioavailability and/or efficacy of the oligonucleotides in vivo.

Aspects of the invention relate to methods for treating a disease associated with heterochromatic down regulation of expression of a gene. In some embodiments, the methods involve administering to a subject an effective amount of an oligonucleotide for increasing expression of the gene, in which the oligonucleotide is complementary to a heterochromatin forming non-coding RNA associated with the gene. In some embodiments, the oligonucleotide is a cleavage promoting oligonucleotide. In some embodiments, the cleavage promoting oligonucleotide is a gapmer. In some embodiments, the cleavage promoting oligonucleotide is an siRNA. In some embodiments, the oligonucleotide is not cleavage promoting (e.g., a mixmer, siRNA, single stranded RNA or double stranded RNA). In certain embodiments, the RNA is a long non-coding RNA (lncRNA). In some embodiments, the lncRNA is antisense to the gene.

In certain embodiments, the gene comprises a repeat region. In some embodiments, the repeat is a triplet repeat. In certain embodiments, the triplet repeat is selected from the group consisting of GAA, CTG, CGG, and CCG. In some embodiments, the repeat is ATTCT. In certain embodiments, the repeat is CCCC.

In some embodiments, the gene is selected from the group consisting of DMPK, CNBP, CSTB, FMR1, AFF2/FMR3, DIP2B, FXN, ATXN10, ATXN8/ATXN8OS, JPH3, and PPP2R2B.

In certain embodiments, the oligonucleotide has the sequence (X₁X₂X₃)ₙ, wherein X is any nucleotide, wherein n is 4-20, wherein the oligonucleotide is 12-60 nucleotides in length. In some embodiments, the oligonucleotide has a terminal flanking sequence.

In certain embodiments, the disease associated with heterochromatin regulation is selected from Angelman syndrome, myotonic dystrophy type 1, Friedreich's ataxia, fragile x syndrome, Prader-Willi syndrome and cancer associated with heterochromatin silencing of tumor suppressor genes.

According to some aspects of the invention methods are provided for treating a disease associated with repeat expansion in a gene. In some embodiments, the methods involve administering to a subject an effective amount of an oligonucleotide for increasing expression of the gene, in which the oligonucleotide is a gapmer that is complementary to a repetitive sequence in a non-coding RNA, the repetitive sequence being a repeating set of nucleotides in which the set is 3-5 nucleotides in length and includes at least 4 repeats. In certain embodiments, the oligonucleotide has the sequence (X₁X₂X₃)ₙ, wherein X is any nucleotide, wherein n is 4-20, wherein the oligonucleotide is 12-60 nucleotides in length. In some embodiments, the oligonucleotide has a terminal flanking sequence. In some embodiments, the RNA is a long non-coding RNA (lncRNA). In certain embodiments, the lncRNA is antisense to the gene. In some embodiments, the repeat is a triplet repeat. In certain embodiments, the triplet repeat is selected from the group consisting of GAA, CTG, CGG, and CCG. In some embodiments, the repeat is ATTCT. In certain embodiments, the repeat is CCCC or CCTG. In some embodiments, the gene is selected from the group consisting of DMPK, CNBP, CSTB, FMR1, AFF2/FMR3, DIP2B, FXN, ATXN10, ATXN8/ATXN8OS, JPH3, and PPP2R2B. In some embodiments, the gene is selected from the group consisting of DMPK, CNBP, CSTB, FMR1, AFF2/FMR3, DIP2B, FXN, and ATXN10.

According to some aspects of the invention, oligonucleotides are provided that comprise (X₁X₂X₃)ₙ, in which X is any nucleotide, in which n is 4-20, in which the oligonucleotide is 12-60 nucleotides in length, and in which the oligonucleotide is cleavage promoting oligonucleotide. In some embodiments, the oligonucleotide includes a terminal flanking sequence. In certain embodiments, the oligonucleotide is a gapmer.

According to some aspects of the invention, a method for treating a disease associated with heterochromatic down regulation of expression of a gene is provide, the method comprising administering to a subject an effective amount of an oligonucleotide for increasing expression of the gene, wherein the oligonucleotide is complementary to a heterochromatin forming non-coding RNA associated with the gene, and wherein the oligonucleotide is a siRNA. In some embodiments, the siRNA is single stranded. In some embodiments, the siRNA is double stranded. In some embodiments, the RNA is a long non-coding RNA (lncRNA). In some embodiments, the lncRNA is antisense to the gene. In some embodiments, the gene comprises a repeat region, optionally wherein the repeat is a triplet repeat. In some embodiments, the triplet repeat is selected from the group consisting of GAA, CTG, CGG, and CCG. In some embodiments, the repeat is ATTCT. In some embodiments, the repeat is CCCC. In some embodiments, the gene is selected from the group consisting of DMPK, CNBP, CSTB, FMR1, AFF2/FMR3, DIP2B, FXN, ATXN10, ATXN8/ATXN8OS, JPH3, and PPP2R2B. In some embodiments, the siRNA has the sequence (X₁X₂X₃)n, wherein X is any nucleotide, wherein n is 4-20, wherein the oligonucleotide is 12-60 nucleotides in length. In some embodiments, the siRNA has a terminal flanking sequence. In some embodiments, the disease associated with heterochromatin regulation is selected from Angelman syndrome, myotonic dystrophy type 1, Friedreich's ataxia, fragile x syndrome, Prader-Willi syndrome and cancer associated with heterochromatin silencing of tumor suppressor genes.

According to other aspects of the invention, a method for treating a disease associated with heterochromatic down regulation of expression of a gene is provided, the method comprising administering to a subject an effective amount of an oligonucleotide for increasing expression of the gene, wherein the oligonucleotide is complementary to a heterochromatin forming non-coding RNA associated with the gene, and wherein the oligonucleotide is a oligonucleotide that does not promote cleavage of the heterochromatin forming non-coding RNA. In some embodiments, the oligonucleotide is a mixmer. In some embodiments, the RNA is a long non-coding RNA (lncRNA). In some embodiments, the lncRNA is antisense to the gene. In some embodiments, the gene comprises a repeat region, optionally wherein the repeat is a triplet repeat. In some embodiments, the triplet repeat is selected from the group consisting of GAA, CTG, CGG, and CCG. In some embodiments, the repeat is ATTCT. In some embodiments, the repeat is CCCC. In some embodiments, the gene is selected from the group consisting of DMPK, CNBP, CSTB, FMR1, AFF2/FMR3, DIP2B, FXN, ATXN10, ATXN8/ATXN8OS, JPH3, and PPP2R2B. In some embodiments, the oligonucleotide has the sequence (X₁X₂X₃)n, wherein X is any nucleotide, wherein n is 4-20, wherein the oligonucleotide is 12-60 nucleotides in length. In some embodiments, the oligonucleotide has a terminal flanking sequence. In some embodiments, the disease associated with heterochromatin regulation is selected from Angelman syndrome, myotonic dystrophy type 1, Friedreich's ataxia, fragile x syndrome, Prader-Willi syndrome and cancer associated with heterochromatin silencing of tumor suppressor genes.

According to other aspects of the invention, an oligonucleotide comprising a sequence as set forth in Table 5 is provided. In some embodiments, the oligonucleotide is 12-60 nucleotides in length.

According to other aspects of the invention, an oligonucleotide comprising at least 8 amino acids of a sequence as set for in Table 5 is provided. In some embodiments, the oligonucleotide is 12-60 nucleotides in length.

The details of one or more embodiments of the invention are set forth in the description below. Other features or advantages of the present invention will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a graph depicting the heterochromatin markers present at different locations along the Frataxin (FXN) gene locus. Heterochromatin-like structures were identified around the repeat region in Friedreich's Ataxia (FRDA) patient cells.

FIG. 2 is diagram depicting the location of a potential RNA transcript in the first intron of FXN based on RNA sequencing data from FRDA patient cells.

FIG. 3 is a diagram depicting the location of RNA transcripts identified using RNA sequencing of RNA from normal cells (GM15851) and cells with high numbers of GAA repeats (GM15850, GM16209, and GM16228). The blue bar indicates the location of RNA transcripts. The arrow underneath each bar indicates the direction of transcription of each RNA transcript.

FIGS. 4A and 4B are a set of graphs depicting the inverse relationship between GAA repeat transcription and FXN mRNA levels as measured in two separate experiments.

FIGS. 5A and 5B are a set of graphs depicting the results of experiments in cells using gapmers specific for the GAA repeat (10 nM or 30 nM). mRNA and protein levels of FXN are shown at days 3, 6, and 9. FIG. 5A shows that treatment of cells with gapmers specific for the GAA repeat increased FXN mRNA levels compared to treatment with a control gapmer to GAPDH. FIG. 5B shows that treatment of cells with gapmers specific for the GAA repeat increased FXN protein levels compared to treatment with a control gapmer to GAPDH or no treatment.

FIGS. 6A and 6B are a set of graphs depicting the results of experiments in cells using gapmers specific for the GAA or TTC repeats (10 nM or 30 nM). mRNA levels of FXN are shown at days 3, 6, and 9. Protein levels of FXN are shown at days 3 and 6. FIG. 6A shows that treatment of cells with gapmers specific for the GAA and TTC repeats increased FXN mRNA levels compared to treatment with a control gapmer to GAPDH. FIG. 6B shows that treatment of cells with gapmers specific for the GAA and TTC repeats increased FXN protein levels compared to treatment with a control gapmer to GAPDH or no treatment.

FIGS. 7A and 7B are a set of graphs depicting the results of experiments in a Friedreich's ataxia mouse model using gapmers specific for GAA repeats (100 mg/kg). mRNA levels of FXN are shown. The FXN RNA levels were normalized to three housekeeper genes (B2M, RPL19 & RPL2). FIG. 7A shows overall averages of FXN mRNA expression for all animals in either the treatment group or the vehicle control group. FIG. 7B shows the values for each animal in the treatment or vehicle control groups as a square, circle, or triangle.

FIG. 8A is a diagram of the FXN gene showing the location of the GAA-repeat in the FXN gene.

FIGS. 8B-8I are a series of graphs showing FXN mRNA levels relative to control wells at day 3 or day 6 post-treatment of cells with oligos designed to target regions flanking the GAA-repeat region.

FIG. 9 is two graphs showing Argonaute (Ago) recruitment within the FXN gene in FRDA diseased (GM15850, GM16209) cells relative to normal (GM15851) cells. The upper graph shows ChIP data obtained using a H3K27me3 antibody. The lower graph shows ChIP data obtained using a Pan-Ago antibody.

### DETAILED DESCRIPTION OF THE INVENTION

Aspects of the invention relate to compositions and methods for increasing expression of genes that have been downregulated or silenced due to heterochromatin formation. In some embodiments, the invention relates to the discovery of non-coding RNAs that induce and/or maintain the heterochromatin state of genes (e.g., mammalian genes) referred to herein as “heterochromatin forming non-coding RNAs”. Such non-coding RNAs are typically expressed from within genomic regions comprising the genes.

Without wishing to be bound by theory, it is believed that in some embodiments these non-coding RNAs generate siRNAs that are incorporated into an RNAi-induced transcriptional silencing (RITS) complex and direct the complex to nascent homologous transcripts expressed from the genes. In some embodiments, this activity of RITS complex leads to recruitment of histone methyltransferases that promote H3K9 methylation and other factors that induce heterochromatin formation at the gene region.

In some embodiments, it has been discovered that oligonucleotides complementary to sequences in a genomic region encoding a heterochromatin forming non-coding RNA are useful for eliminating or reversing heterochromatin at the gene and thereby activating or inducing expression of the gene. In some embodiments, the oligonucleotides are complementary to a sequence of the heterochromatin forming non-coding RNA. In some embodiments, the oligonucleotides are complementary to the reverse complement of a sequence of the heterochromatin forming non-coding RNA. In some embodiments, the oligonucleotides inhibit formation of endogenous siRNAs that are incorporated into a RITS complex and direct the complex to nascent homologous transcripts expressed from the genes and thereby prevent the formation or maintenance of heterochromatin at the genes. Accordingly, in some embodiments, methods are provided for inducing gene expression that involve delivering to a cell an effective amount of an oligonucleotide complementary to sequence in a genomic region encoding a heterochromatin forming non-coding RNA.

In some embodiments, the non-coding RNA is a long non-coding RNA (lncRNA). In some embodiments, the lncRNA is a singled-stranded or double-stranded. In some embodiments, the sequence of the non-coding RNA is sense relative to the gene that it regulates. In some embodiments, the sequence of the non-coding RNA is antisense relative to the gene that it regulates. In some embodiments, the non-coding RNA is expressed from a genomic region corresponding to a non-coding portion of the gene that it regulates. In some embodiments, the non-coding portion is a promoter, intron, 3′ UTR or 5′ UTR or an upstream or downstream regulatory region. In some embodiments, the non-coding RNA is expressed from a genomic region corresponding to a coding portion (e.g., an exon) of the gene that it regulates. However, it should be appreciated that the methods are not limited to modulating the heterochromatin state of protein coding genes. In some embodiments, the methods may be used to modulate the heterochromatin state of non-protein coding genes (e.g., lncRNAs, miRNAs, etc.)

In some embodiments, a gene regulated by a heterochromatin forming non-coding RNA comprises a triplet repeat region or other repeat sequences (e.g., Alu Repeats, mammalian-wide interspersed repeats, LINEs, SINEs, etc.). In some embodiments, the triplet repeat is selected from the group consisting of GAA, CTG, CGG, and CCG.

In some embodiments, the heterochromatin forming non-coding RNA comprises a sequence that is encoded from within a repeat region of a gene that it regulates. According, in some embodiments, the heterochromatin forming non-coding RNAs comprise triplet repeat sequences. In some embodiments, heterochromatin forming non-coding RNAs comprising triplet repeat sequences are expressed at high levels or are highly active when the number of repeats exceeds a certain threshold (e.g., greater than 25 or more repeats). Therefore, in some embodiments, expression of a gene is reduced or silenced as a result of heterochromatin formation in cells that have an triplet repeat or other repetitive sequence that exceeds a certain length threshold. In some embodiments, the length of the repeat is 10 to 50 repeats, 25 to 100 repeats, 50 to 150 repeats, 100 to 500 repeats, 100 to 1000 repeats or more. In some embodiments, the length of the repeat is at least 10, at least 25, at least 50, at least 100, at least 150, at least 250, at least 500 or more.

Oligonucleotides disclosed herein may target the repeat region or a sequence occurring at a position adjacent to the repeat region. In some embodiments, the oligonucleotide targets a region within 10, 20, 30, 40, 50, 100, 200, 300, 400, 500 or more nucleotides from an end of the repeat region. In some embodiments, oligonucleotides may have a portion targeting a repeat region and a portion targeting an adjacent non-repeat region. Such oligonucleotides may be useful for selectively targeting genes that have repeat regions, whereby the portion of the oligonucleotide that does not target the repeat is a gene specific portion of sufficient length and sequence complexity so as to confer target specific on the oligonucleotide. Such oligonucleotides may be particularly advantageous where the repeat region occurs elsewhere within the genome of a cell harboring the gene.

In some embodiments, an oligonucleotide disclosed herein targets a region within 100 kb, 50 kb, 10 kb, or 5 kb from the end of a repeat region (e.g., a repeat region of FXN). In some embodiments, the oligonucleotide targets a region within 5 kb from the end of a repeat region of FXN (e.g., a repeat region within the 1st intron of FXN). In some embodiments, the oligonucleotide targets one or more of the regions listed below (SEQ ID NOs: 63-68), which are the plus and minus strands of a repeat region of FXN located within the 1st intron of FXN as well as the flanking regions of the repeat region (SEQ ID NOs: 63 and 64, respectively) and the plus and minus strands of the flanking regions alone (SEQ ID NOs: 65-68). In some embodiments, the oligonucleotide comprises a sequence as set forth in Table 5, or a fragment thereof. In some embodiments, the region of complementarity of an oligonucleotide is complementary with at least 5 to 15, 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of one or both of the sequences listed below (SEQ ID NOs: 63-68). In some embodiments, the region of complementarity is complementary with at least 5 or at least 8 consecutive nucleotides of one or both of the sequences listed below (SEQ ID NOs: 63-68). The oligonucleotide may be at least 80% complementary to (optionally one of at least 85%, 90%, 91%, 92%, 93%, 94%, 95%, 96%, 97%, 98%, 99% or 100% complementary to) the consecutive nucleotides of one or both of the sequences listed below (SEQ ID NOs: 63-68). In some embodiments the oligonucleotide may contain 1, 2 or 3 base mismatches compared to the portion of the consecutive nucleotides of one or both of the sequences listed below (SEQ ID NOs: 63-68). In some embodiments the oligonucleotide may have up to 3 mismatches over 15 bases, or up to 2 mismatches over 10 bases.

>hg19_dna range = chr9:
71647062-71657262, strand = +

(SEQ ID NO: 63)

AAAAAAAAAAAGAGAGAGAGAGGGAGTTAGAAGGAAGATGCATCATTTTT
ATGACCTGGACTTGGAAGTCACCAAGCAGCACTTCTGCAGTACCCTGTTG
GTTGGAATAGTTGTAGCCCAAACCCGAATTCGAAGGGAGGAGAATAGATA
ACATCCCTGGGTGACAGGAATGTCAAAGTCCCAAACAGCATATGACATGT
GACAAATATTGGTGTGGCCTTCTTTGGAAGATCCAATCTTCCATACCAGG
CAAAGGGATGGAAGACTAAGGAACAACATGAGGGATAGCCAGAGAGGGAA
AAAGCATCACTTGTTCTAGGAACTACAAATAGCTTGAAGAAGCAAAGATG
TCTAGATGCCTCCCAATATGCAGAGTGGGGTGTACAGAAGAGAGTGGTAA
GGGCGCTGGGAGAGCTAAGGTGGGCAAGAGAGCTTCCTCTGTCATGCTAA
GAAAGTTGGAATTTATCTTGATGGTGGTGAAAGCAGAGGGCTATGGTTAG
ATTCACATTTGAGATTTAGATTTTTAGATTTAAAATGATCACCCTGGTGA
CACTGGCTTAACTCACAATTTTGCCCAAGGCCTATGCTACCACAGTGCTT
CTGAAACTTTAAAGCACATTAGAATCACCTGGAGGTCTTGTTAAACCATG
GATTGCTGGGCCTTGAAACCCCAGAGATTCTGATTCAGTAGATCGAGAAT
AGGGCCTGAGAATTTGTATTTCTAACAAGTTTCCAGGTGATGCTGAGGCT
GCTGGCCCAGCGACCACATTTGATAATCATAGCCCTCTGATAAATCCTAT
CAAAATATCCTAATGGCAGAGCAAGGGAATTCTGGTGATATCCTCCCCTA
CCCATAACCTGACAGCTATTAGGATCTGCCTACTTGAGGCTAAAAGCAAC
CAAGAGAGGAACAGCTACAGTGTACCACAGAGTCCCTCAACATCTTTGCC
CACGCCACGGTGCCCCAGCTTCTTACCAAGTGTGCCTGATTCCTCTTGAC
TACCTCCAAGGAAGTGGAGAAAGACAAGTTCTTGCGAAGCCTTCGTCTTC
TCTGATATGCTATTCTATGTCTATTTCTTTGGCCAAAAAGATGGGGCAAT
GATATCAACTTTGCAGGGAGCTGGAGCATTTGCTAGTGACCTTTCTATGC
CAGAACTTGCTAAGCATGCTAGCTAATAATGATGTAGCACAGGGTGCGGT
GGCTCACGCCTGTAATCTCAGCACTTTGGGCGGCCGAGGCGGGCGGATCA
CCTGAGGTCAGGAGTTCGAGACCAGCCTGGCCAACATGATGAAACCCCAT
CTCTACTAAAAATACAAAAATTAGCCAGGCGTGGTGGTGGGCACCTGCAA
TCCCAGCTACTCTGGAGGCTGAGACAGAATCTCTTGAACCCAGGAGGTGG
AGATTGCAGTGAGCAGAGATGGCACCACTGCATTCCAGCCTGGGCAACAA
AGCAAGACTCTGTCTCAAATAATAATAATAATAATAACTAATGATGCAGC
TTTCTCTCTCTGAGTATATAATGCAGTTCTGATGATGTGAGGAAGGGCCT
CACTGTTGGTGTGGCAGAGTCTGAGACCATGGCTGGCAATGAAAACACTA
CCCTTTGATGCCTATGGGCTCTCCCTTTATGGTTTCAAGGAGGGCTTCTC
AATCTTGGCAGAATTTTGGACTGGATAGTTCTTTGTTGCACAGGTGGGGG
GCTGTCCTGCACATCACAGGATGTTTCATCCCTGGCCTCTACCTACTAGA
TGCCAGTAGAACATACCCACCCCACAGCTGCCTGTTGTGACAATCAAAAG
CATCTCCAGATACTTTGCAGGGGGAAAATGATTTCTCCAGGCCTGGCATA
TACATAACAGTATTTAAGCAGCTGCCTAGAATTAATTAAACACAGAAGGA
TGTCTCTCATCCAGAATGCCCTGGACCACCTCTTTGATAGGCAATCAGAT
CCCACCTCCTCCACCCTATTTTTGAAGGCCCTGTGCCAACACCACTTCTT
CCATGAATACTTCCTTGATTCCCCCATCCCTAGCTCTATATAAATCTCCC
ACTCAACACTCACACCTGTTAGTTTACATTCCTCTTGACACTTGTCATTT
AGCATCCTAAGTATGTAAACATGTCTCTCTTCACGATTCACAAAGTGGCT
TTGGAAGAACTTTAGTACCTTCCCATCTTCTCTGCCATGGAAAGTGTACA
CAACTGACATTTTCTTTTTTTTTAAGACAGTATCTTGCTATGATGGCCGG
GCTGGAATGCTGTGGCTATTCACAGGCACAATCATAGCTCACTGCAGCCT
TGAGCTCCCAGGCTCAAGTGATCCTCCCGCCTCAGCCTCCTGAGTAGCTG
AGATCACAGGCATGCACTACCACACTCGGCTCACATTTGACATCCTCTAA
AGCATATATAAAATGTGAAGAAAACTTTCACAATTTGCATCCCTTTGTAA
TATGTAACAGAAATAAAATTCTCTTTTAAAATCTATCAACAATAGGCAAG
GCACGGTGGCTCACGCCTGTCGTCTCAGCACTTTGTGAGGCCCAGGCGGG
CAGATCGTTTGAGCCTAGAAGTTCAAGACCACCCTGGGCAACATAGCGAA
ACCCCCTTTCTACAAAAAATACAAAAACTAGCTGGGTGTGGTGGTGCACA
CCTGTAGTCCCAGCTACTTGGAAGGCTGAAATGGGAAGACTGCTTGAGCC
CGGGAGGGAGAAGTTGCAGTAAGCCAGGACCACACCACTGCACTCCAGCC
TGGGCAACAGAGTGAGACTCTGTCTCAAACAAACAAATAAATGAGGCGGG
TGGATCACGAGGTCAGTAGATCGAGACCATCCTGGCTAACACGGTGAAAC
CCGTCTCTACTAAAAAAAAAAAAAAATACAAAAAATTAGCCAGGCATGGT
GGCGGGCGCCTGTAGTCCCAGTTACTCGGGAGGCTGAGGCAGGAGAATGG
CGTGAAACCGGGAGGCAGAGCTTGCAGTGAGCCGAGATCGCACCACTGCC
CTCCAGCCTGGGCGACAGAGCGAGACTCCGTCTCAATCAATCAATCAATC
AATAAAATCTATTAACAATATTTATTGTGCACTTAACAGGAACATGCCCT
GTCCAAAAAAAACTTTACAGGGCTTAACTCATTTTATCCTTACCACAATC
CTATGAAGTAGGAACTTTTATAAAACGCATTTTATAAACAAGGCACAGAG
AGGTTAATTAACTTGCCCTCTGGTCACACAGCTAGGAAGTGGGCAGAGTA
CAGATTTACACAAGGCATCCGTCTCCTGGCCCCACATACCCAACTGCTGT
AAACCCATACCGGCGGCCAAGCAGCCTCAATTTGTGCATGCACCCACTTC
CCAGCAAGACAGCAGCTCCCAAGTTCCTCCTGTTTAGAATTTTAGAAGCG
GCGGGCCACCAGGCTGCAGTCTCCCTTGGGTCAGGGGTCCTGGTTGCACT
CCGTGCTTTGCACAAAGCAGGCTCTCCATTTTTGTTAAATGCACGAATAG
TGCTAAGCTGGGAAGTTCTTCCTGAGGTCTAACCTCTAGCTGCTCCCCCA
CAGAAGAGTGCCTGCGGCCAGTGGCCACCAGGGGTCGCCGCAGCACCCAG
CGCTGGAGGGCGGAGCGGGCGGCAGACCCGGAGCAGCATGTGGACTCTCG
GGCGCCGCGCAGTAGCCGGCCTCCTGGCGTCACCCAGCCCAGCCCAGGCC
CAGACCCTCACCCGGGTCCCGCGGCCGGCAGAGTTGGCCCCACTCTGCGG
CCGCCGTGGCCTGCGCACCGACATCGATGCGACCTGCACGCCCCGCCGCG
CAGTAAGTATCCGCGCCGGGAACAGCCGCGGGCCGCACGCCGCGGGCCGC
ACGCCGCACGCCTGCGCAGGGAGGCGCCGCGCACGCCGGGGTCGCTCCGG
GTACGCGCGCTGGACTAGCTCACCCCGCTCCTTCTCAGGGCGGCCCGGCG
GAAGCGGCCTTGCAACTCCCTTCTCTGGTTCTCCCGGTTGCATTTACACT
GGCTTCTGCTTTCCGAAGGAAAAGGGGACATTTTGTCCTGCGGTGCGACT
GCGGGTCAAGGCACGGGCGAAGGCAGGGCAGGCTGGTGGAGGGGACCGGT
TCCGAGGGGTGTGCGGCTGTCTCCATGCTTGTCACTTCTCTGCGATAACT
TGTTTCAGTAATATTAATAGATGGTATCTGCTAGTATATACATACACATA
ATGTGTGTGTCTGTGTGTATCTGTATATAGCGTGTGTGTTGTGTGTGTGT
GTTTGCGCGCACGGGCGCGCGCACACCTAATATTTTCAAGGCTGGATTTT
TTTGAACGAAATGCTTTCCTGGAACGAGGTGAAACTTTCAGAGCTGCAGA
ATAGCTAGAGCAGCAGGGGCCCTGGCTTTTGGAAACTGACCCGACCTTTA
TTCCAGATTCTGCCCCACTCCGCAGAGCTGTGTGACCTTGGGGGATTCCC
CTAACCTCTCTGAGACGTGGCTTTGTTTTCTGTAGGGAGAAGATAAAGGT
GACGCCCATTTTGCGGACCTGGTGTGAGGATTAAATGGGAATAACATAGA
TAAAGTCTTCAGAACTTCAAATTAGTTCCCCTTTCTTCCTTTGGGGGGTA
CAAAGAAATATCTGACCCAGTTACGCCACGGCTTGAAAGGAGGAAACCCA
AAGAATGGCTGTGGGGATGAGGAAGATTCCTCAAGGGGAGGACATGGTAT
TTAATGAGGGTCTTGAAGATGCCAAGGAAGTGGTAGAGGGTGTTTCACGA
GGAGGGAACCGTCTGGGCAAAGGCCAGGAAGGCGGAAGGGGATCCCTTCA
GAGTGGCTGGTACGCCGCATGTATTAGGGGAGATGAAAGAGGCAGGCCAC
GTCCAAGCCATATTTGTGTTGCTCTCCGGAGTTTGTACTTTAGGCTTGAA
CTTCCCACACGTGTTATTTGGCCCACATTGTGTTTGAAGAAACTTTGGGA
TTGGTTGCCAGTGCTTAAAAGTTAGGACTTAGAAAATGGATTTCCTGGCA
GGACGCGGTGGCTCATGCCCATAATCTCAGCACTTTGGGAGGCCTAGGAA
GGTGGATCACCTGAGGTCCGGAGTTCAAGACTAACCTGGCCAACATGGTG
AAACCCAGTATCTACTAAAAAATACAAAAAAAAAAAAAAAAGAAGAAGAA
GAAGAAGAAAATAAAGAAAAGTTAGCCGGGCGTGGTGTCGCGCGCCTGTA
ATCCCAGCTACTCCAGAGGCTGCGGCAGGAGAATCGCTTGAGCCCGGGAG
GCAGAGGTTGCATTAAGCCAAGATCGCCCAATGCACTCCGGCCTGGGCGA
CAGAGCAAGACTCCGTCTCAAAAAATAATAATAATAAATAAAAATAAAAA
ATAAAATGGATTTCCCAGCATCTCTGGAAAAATAGGCAAGTGTGGCCATG
ATGGTCCTTAGATCTCCTCTAGGAAAGCAGACATTTATTACTTGGCTTCT
GTGCACTATCTGAGCTGCCACGTATTGGGCTTCCACCCCTGCCTGTGTGG
ACAGCATGGGTTGTCAGCAGAGTTGTGTTTTGTTTTGTTTTTTTGAGACA
GAGTTTCCCTCTTGTTGCCCAGGCTGGAGTGCAGTGGCTCAGTCTCAGCT
CACTGCAACCTCTGCCTCCTGGGTTCAAGTGATTCTCCTGCCTCAGCCTC
CCGAGTAGCTGGGATTATCGGCTAATTTTGTATTTTTAGTAGAGACAGAT
TTCTCCATGTTGGTCAGGCTGGTCTCGAACTCCCAACCTCAGGTGATCCG
CCCACCTCGCCCTCCCAAAGTGCTGGAATTACAGGCGTGAGCCACCGCGT
CTGGCCATCAGCAGAGTTTTTAATTTAGGAGAATGACAAGAGGTGGTACA
GTTTTTTAGATGGTACCTGGTGGCTGTTAAGGGCTATTGACTGACAAACA
CACCCAACTTGGCGCTGCCGCCCAGGAGGTGGACACTGGGTTTCTGGATA
GATGGTTAGCAACCTCTGTCACCAGCTGGGCCTCTTTTTTTCTATACTGA
ATTAATCACATTTGTTTAACCTGTCTGTTCCATAGTTCCCTTGCACATCT
TGGGTATTTGAGGAGTTGGGTGGGTGGCAGTGGCAACTGGGGCCACCATC
CTGTTTAATTATTTTAAAGCCCTGACTGTCCTGGATTGACCCTAAGCTCC
CCCTGGTCTCCAAAATTCATCAGAAACTGAGTTCACTTGAAGGCCTCTTC
CCCACCCTTTTCTCCACCCCTTGCATCTACTTCTAAAGCAGCTGTTCAAC
AGAAACAGAATGGGAGCCACACACATAATTCTACATTTTCTAGTTAAAAA
GAAAAAAAAATCATTTTCAACAATATATTTATTCAACCTAGTACATACAA
AATATTATCATTCCAACATGTAATCAGTATTTTAAAAATCAGTAATGAGA
CCAGGCACGGTGGCTCACGACTGTAATCCCAGGACTTTGGGAGGCCGAGG
CGAGTGGATCATCTGAGATCAGGAGTTCAAGACCAGCCTGGCCAACATGG
TGAAACCCCATCTCTACTAAAAACTAGCTCAGCATGGTGGTGGGTGCCTG
TAGTCCCAGCTACTCGGGAGGCTGAGGCATGAGAATCACTTGAGCCCAGG
AGGCAGAGGTTGCAGTGAGCCAAGATTTTGGGGGATTCTGTGACATACAA
AAAAAATCAGTAATAAGATATCTTGCATACTCTTTTCGTACTCATATACT
TCCAGCATATCTCAATTCACAATTTCTAAGTAAATGCTCTATCTGTATTT
ACTTTTATAAAATTCACAATTAAAAATGAAGGTTCACATAGTCAAGTTGT
TCCAAACACACTTAAATGTCTCCTAGGCTGGGTGTGGTTGCTCACACCTG
TAATCCCAGCACTTTGGGAGGCTGAGATGGGCGGATCACCTGAGGTCAGG
AGTTTGAGACCAGCCTGGCCAACATGGTGAAACCCCGTCTCTACTAAAAA
TACAAAAATTAGCTGGATGTGGTGGCACTCACCTGTAATCCCAGCTACTC
AGGAGGCTGAGGCAGGATAATTGCTTGAACCCGGGAGGTGGTGGAGGTTG
CAGTGAGCCGAGATCGCACCACTGCCTTCCAACCTGGGCGACAGAGCGAG
ACTCCGTCTCAAAAAAAAAAAAAAGGCTCCTAATAACTTTATTACTTTAT
TATCACCTCAAATAATTAAAATTAAATGAAGTTGAAAATCCAGGTCCTCA
GTCCCATTAGCCACATTTCTAGTGCTCAGTAGCCACGGGGGCTGGTGACC
ACCACATGGGACAGCATATTTAGTACCTGATCATTGGTTCTCAGATCTGG
CTACTCAGCAGAACCAAGAATCCACAGAAACGGCTTTTAAAAGCACAGCC
CCACAGCCCCCAGCCCCAGCCTTACCTACCTGGAGGCTGGGAAGGACTCT
GATTCCACGAGGCAGCCTATGTTTTTTGATGGAGGGATGTGACAGGGGCT
GCATCTTTAACGTTTCCTCTTAAATACTGGAGACAGCTTCGAGGAGGAGA
TAACTGGATGTGTCTTAGTCCATTTGATGGAGGGATGTGACGGGGCTGCG
TCTTTAACGTTTCCTCTTAAATACCGGAGACAGCTTCGAGAAGGAGATAA
CTGGATGTTTCTTAGTCCATTTTCTGTTGCTTGTGACAGAATACCTGAAA
CTGGGCAATTTATATGGTAAAAAATTTTCTTCTTACTGCTCTGGAGGCTG
AGAAGTCCAAAGTCAAGTCCCTTCTTGCTGGTGGGGACTTTGCAGAGTAT
TGAGGCGGCACCGGGCGTCATATGGTAAGGGGCTGAGTGTGCTACCTCAG
GTGTCTTTTTCTTTTCTTATAAAGCCTAACTAGTTTCACTCCCATGATAA
CCCATTAATCTATGAATGGATTAATCCATTATTGAGGGAAGAACCTTCAT
GACCCAGTCACCGCTTAAAGGCCCCACCTCTCAATACTGCCACATCGGGA
ATTAAGTTTCAACATGAGTTTCGGAGGTGACAAACATTCAAACCATAGCA
TGCTGTCTCTTAAATGACTCAATAAGCTCCTGTGGCATCCACTTCTGCAT
GCCTTGGGCAGCTTTTAGACATCTGTCCATTTTCCTAGAGGGACAAGACC
ACCACCTGTGATCCTATGACCTTTTGGCTTTAGGCCTAACAAGCAGGTTA
TACCCTCACTCACTTTCAAATCATTTTTATTGTCTTGCAGACAATTTACA
CAAGTTTACACATAGAAAAGGATATGTAAATATTTATACGCTGCCGGGCG
CGGTGGCTCACGCCTGTAATCCCAGCACTTTGGGAGGCCGAGGCAGGTGG
ATCACGAGTTCAGGAGATGGAGACCATCCTGGCTAATACGATGAAACCCC
ATCTCTACTAAAAATACAAAAAATTAGCCGGGCGTGGTGACGGGTGCCTG
TAGTCCCCACTACTCGGGACGCTGAGGCAGGAGAATGGCGTGAACCCGGG
AGGCAGAGCTTGCAGTGATCCGAGATCGTGCCACTGCACTCCAGCCTGGG
TGACAGAGCGAGACTGCATCTCAAAGAAAAAAATAAATAAATAAATAAAT
ATTTATACTGCTTATAAACTAATAATAAATGCTATGGTCTGCATGTTTGT
GTCACCCCACCATTCATATGTTAAAACCTAATCACCAAAGTGATATTAGG
AGGTGGGGCCCTTGGGAGGTGATGAGGTATGAGGGTGGAGCCCATATGAT
TGGGATTAGTGCCCTTCTAAAATAGCCCAACGGAGCCCAGTGACAAGGCA
TCATCTATGAACCAGGAAACTGGCCCTCACCAGACACCAAAGCTGTTGGT
GCATTGATCTTGGATTTCCCACCCTCCAGGACTCTAAGAAACACATTTCT
ATTGTTTATAAGCCACCCAGTGGCTGGTATTTTGTTATAACATCCCAGAC
TAAGACAAATAACAAATACTTGTATCCCTGACACCAGGTTAAGAGATAGA
ATTTGTTTGTTCCTCTGGAGGCCCTTGTCTTCACCCCATCACTGCCCTGT
CCTCCCTGGAGGAATCTGCCAGCCCGAATTCTGTTCATCGTACCCTCCTT
TTCTTAGAGTTTGACCTCCTCTGTATCTCCCCCAATCCATGTATTGCTTA
TATACAAGGTATTCTGCTGTATCTGTTCTGCTATGGCTTGCCCCTTTTGT
TCAACACTGTTTTTGTGCGTCATCTGCATTGATGCATGCAGTTGTCCTTT
ATTTGTTCTCACTGCTGGATAGTATCTGGTTGGGTAAATATATCACACTG
TAAATCACACTATCCAGGTTCCTTTAGGTGACATTTGGTTGATTGCAGTG
TTCTGTTGTTACGATGGTGCTGCTGTGACTGTTCTTGTGCATGGACAGAA
GTTCCTTTCAGGTGAATTTCTCAGAATGGAATTGCTGGGCAAAGGGGCAG
CCAATAATCAACTCATTTGATGCCAAAAGTGGTGGTGCCAGTTCATCCTC
CCCTGCGAGGTATGGGTCCTGATTCACTCTTCAAGTGCTGTGGTTTGACA
GGGCCGGGGGTGACAAGGGGACACCTGGGAAGGAAAGCTGGGCTCCCTGC
TGGCCATCCAGGCCAGTCCTTACCAGGGGGTAGGCAATGATTGGGTCAAG
TGGTTCCTGACCACTGGGCCTGAGACTTCAGGCCCAGAAACTATCTAATA
TTTCCTCAAATGCATCCCATGAGCAGGCACTGTGTGAGTGAGCACACACA
TCTGAAGCCTCAAGCTAGGCAAGCCTACCATGACTTGTGGTCCAAGGGCT
CACGGGTGACCTGGAGTTAGAGGGAGACATGGCTGCCAGGTGGCTTTAGA
AAGAACACTCATCATGGCCAGGTGCGGTGGCTTACGCCTGTAATCCCAGC
ACTTTGGGAGGCCAAGGTGGGTGGATCATGAGGTCAGGAGTGAGACCAGC
CTGACCAACATGCTGAAACCTGTCTCTCCTAAAAACACAAAAATTAGCTG
GGCATGGAGGTGCACGCCTGTAATCCCAGCTACTCAGGAGGCTGAGGCAG
GAGAATCACTTGAACCCGGGAGGCGGAGGTTGCAATAAGCCTAGATTGTG
CCACTGCATTCCAGCCTGGGCAACAGAGCAAGACTCCGTCTCAGAAAAAA
AAAAAAAAAGGAAGAACACTCATCCTATGACCTTGACCTCCAAGCTTTGC
CTCCCTCAAGCAGAACAGAATGGAGCCTCCCTTAGGCAGAGGCGGAAGTT
T
>hg19_dna range = chr9:
71647062-71657262, strand = −

(SEQ ID NO: 64)

AAACTTCCGCCTCTGCCTAAGGGAGGCTCCATTCTGTTCTGCTTGAGGGA
GGCAAAGCTTGGAGGTCAAGGTCATAGGATGAGTGTTCTTCCTTTTTTTT
TTTTTTTCTGAGACGGAGTCTTGCTCTGTTGCCCAGGCTGGAATGCAGTG
GCACAATCTAGGCTTATTGCAACCTCCGCCTCCCGGGTTCAAGTGATTCT
CCTGCCTCAGCCTCCTGAGTAGCTGGGATTACAGGCGTGCACCTCCATGC
CCAGCTAATTTTTGTGTTTTTAGGAGAGACAGGTTTCAGCATGTTGGTCA
GGCTGGTCTCACTCCTGACCTCATGATCCACCCACCTTGGCCTCCCAAAG
TGCTGGGATTACAGGCGTAAGCCACCGCACCTGGCCATGATGAGTGTTCT
TTCTAAAGCCACCTGGCAGCCATGTCTCCCTCTAACTCCAGGTCACCCGT
GAGCCCTTGGACCACAAGTCATGGTAGGCTTGCCTAGCTTGAGGCTTCAG
ATGTGTGTGCTCACTCACACAGTGCCTGCTCATGGGATGCATTTGAGGAA
ATATTAGATAGTTTCTGGGCCTGAAGTCTCAGGCCCAGTGGTCAGGAACC
ACTTGACCCAATCATTGCCTACCCCCTGGTAAGGACTGGCCTGGATGGCC
AGCAGGGAGCCCAGCTTTCCTTCCCAGGTGTCCCCTTGTCACCCCCGGCC
CTGTCAAACCACAGCACTTGAAGAGTGAATCAGGACCCATACCTCGCAGG
GGAGGATGAACTGGCACCACCACTTTTGGCATCAAATGAGTTGATTATTG
GCTGCCCCTTTGCCCAGCAATTCCATTCTGAGAAATTCACCTGAAAGGAA
CTTCTGTCCATGCACAAGAACAGTCACAGCAGCACCATCGTAACAACAGA
ACACTGCAATCAACCAAATGTCACCTAAAGGAACCTGGATAGTGTGATTT
ACAGTGTGATATATTTACCCAACCAGATACTATCCAGCAGTGAGAACAAA
TAAAGGACAACTGCATGCATCAATGCAGATGACGCACAAAAACAGTGTTG
AACAAAAGGGGCAAGCCATAGCAGAACAGATACAGCAGAATACCTTGTAT
ATAAGCAATACATGGATTGGGGGAGATACAGAGGAGGTCAAACTCTAAGA
AAAGGAGGGTACGATGAACAGAATTCGGGCTGGCAGATTCCTCCAGGGAG
GACAGGGCAGTGATGGGGTGAAGACAAGGGCCTCCAGAGGAACAAACAAA
TTCTATCTCTTAACCTGGTGTCAGGGATACAAGTATTTGTTATTTGTCTT
AGTCTGGGATGTTATAACAAAATACCAGCCACTGGGTGGCTTATAAACAA
TAGAAATGTGTTTCTTAGAGTCCTGGAGGGTGGGAAATCCAAGATCAATG
CACCAACAGCTTTGGTGTCTGGTGAGGGCCAGTTTCCTGGTTCATAGATG
ATGCCTTGTCACTGGGCTCCGTTGGGCTATTTTAGAAGGGCACTAATCCC
AATCATATGGGCTCCACCCTCATACCTCATCACCTCCCAAGGGCCCCACC
TCCTAATATCACTTTGGTGATTAGGTTTTAACATATGAATGGTGGGGTGA
CACAAACATGCAGACCATAGCATTTATTATTAGTTTATAAGCAGTATAAA
TATTTATTTATTTATTTATTTTTTTCTTTGAGATGCAGTCTCGCTCTGTC
ACCCAGGCTGGAGTGCAGTGGCACGATCTCGGATCACTGCAAGCTCTGCC
TCCCGGGTTCACGCCATTCTCCTGCCTCAGCGTCCCGAGTAGTGGGGACT
ACAGGCACCCGTCACCACGCCCGGCTAATTTTTTGTATTTTTAGTAGAGA
TGGGGTTTCATCGTATTAGCCAGGATGGTCTCCATCTCCTGAACTCGTGA
TCCACCTGCCTCGGCCTCCCAAAGTGCTGGGATTACAGGCGTGAGCCACC
GCGCCCGGCAGCGTATAAATATTTACATATCCTTTTCTATGTGTAAACTT
GTGTAAATTGTCTGCAAGACAATAAAAATGATTTGAAAGTGAGTGAGGGT
ATAACCTGCTTGTTAGGCCTAAAGCCAAAAGGTCATAGGATCACAGGTGG
TGGTCTTGTCCCTCTAGGAAAATGGACAGATGTCTAAAAGCTGCCCAAGG
CATGCAGAAGTGGATGCCACAGGAGCTTATTGAGTCATTTAAGAGACAGC
ATGCTATGGTTTGAATGTTTGTCACCTCCGAAACTCATGTTGAAACTTAA
TTCCCGATGTGGCAGTATTGAGAGGTGGGGCCTTTAAGCGGTGACTGGGT
CATGAAGGTTCTTCCCTCAATAATGGATTAATCCATTCATAGATTAATGG
GTTATCATGGGAGTGAAACTAGTTAGGCTTTATAAGAAAAGAAAAAGACA
CCTGAGGTAGCACACTCAGCCCCTTACCATATGACGCCCGGTGCCGCCTC
AATACTCTGCAAAGTCCCCACCAGCAAGAAGGGACTTGACTTTGGACTTC
TCAGCCTCCAGAGCAGTAAGAAGAAAATTTTTTACCATATAAATTGCCCA
GTTTCAGGTATTCTGTCACAAGCAACAGAAAATGGACTAAGAAACATCCA
GTTATCTCCTTCTCGAAGCTGTCTCCGGTATTTAAGAGGAAACGTTAAAG
ACGCAGCCCCGTCACATCCCTCCATCAAATGGACTAAGACACATCCAGTT
ATCTCCTCCTCGAAGCTGTCTCCAGTATTTAAGAGGAAACGTTAAAGATG
CAGCCCCTGTCACATCCCTCCATCAAAAAACATAGGCTGCCTCGTGGAAT
CAGAGTCCTTCCCAGCCTCCAGGTAGGTAAGGCTGGGGCTGGGGGCTGTG
GGGCTGTGCTTTTAAAAGCCGTTTCTGTGGATTCTTGGTTCTGCTGAGTA
GCCAGATCTGAGAACCAATGATCAGGTACTAAATATGCTGTCCCATGTGG
TGGTCACCAGCCCCCGTGGCTACTGAGCACTAGAAATGTGGCTAATGGGA
CTGAGGACCTGGATTTTCAACTTCATTTAATTTTAATTATTTGAGGTGAT
AATAAAGTAATAAAGTTATTAGGAGCCTTTTTTTTTTTTTTGAGACGGAG
TCTCGCTCTGTCGCCCAGGTTGGAAGGCAGTGGTGCGATCTCGGCTCACT
GCAACCTCCACCACCTCCCGGGTTCAAGCAATTATCCTGCCTCAGCCTCC
TGAGTAGCTGGGATTACAGGTGAGTGCCACCACATCCAGCTAATTTTTGT
ATTTTTAGTAGAGACGGGGTTTCACCATGTTGGCCAGGCTGGTCTCAAAC
TCCTGACCTCAGGTGATCCGCCCATCTCAGCCTCCCAAAGTGCTGGGATT
ACAGGTGTGAGCAACCACACCCAGCCTAGGAGACATTTAAGTGTGTTTGG
AACAACTTGACTATGTGAACCTTCATTTTTAATTGTGAATTTTATAAAAG
TAAATACAGATAGAGCATTTACTTAGAAATTGTGAATTGAGATATGCTGG
AAGTATATGAGTACGAAAAGAGTATGCAAGATATCTTATTACTGATTTTT
TTTGTATGTCACAGAATCCCCCAAAATCTTGGCTCACTGCAACCTCTGCC
TCCTGGGCTCAAGTGATTCTCATGCCTCAGCCTCCCGAGTAGCTGGGACT
ACAGGCACCCACCACCATGCTGAGCTAGTTTTTAGTAGAGATGGGGTTTC
ACCATGTTGGCCAGGCTGGTCTTGAACTCCTGATCTCAGATGATCCACTC
GCCTCGGCCTCCCAAAGTCCTGGGATTACAGTCGTGAGCCACCGTGCCTG
GTCTCATTACTGATTTTTAAAATACTGATTACATGTTGGAATGATAATAT
TTTGTATGTACTAGGTTGAATAAATATATTGTTGAAAATGATTTTTTTTT
CTTTTTAACTAGAAAATGTAGAATTATGTGTGTGGCTCCCATTCTGTTTC
TGTTGAACAGCTGCTTTAGAAGTAGATGCAAGGGGTGGAGAAAAGGGTGG
GGAAGAGGCCTTCAAGTGAACTCAGTTTCTGATGAATTTTGGAGACCAGG
GGGAGCTTAGGGTCAATCCAGGACAGTCAGGGCTTTAAAATAATTAAACA
GGATGGTGGCCCCAGTTGCCACTGCCACCCACCCAACTCCTCAAATACCC
AAGATGTGCAAGGGAACTATGGAACAGACAGGTTAAACAAATGTGATTAA
TTCAGTATAGAAAAAAAGAGGCCCAGCTGGTGACAGAGGTTGCTAACCAT
CTATCCAGAAACCCAGTGTCCACCTCCTGGGCGGCAGCGCCAAGTTGGGT
GTGTTTGTCAGTCAATAGCCCTTAACAGCCACCAGGTACCATCTAAAAAA
CTGTACCACCTCTTGTCATTCTCCTAAATTAAAAACTCTGCTGATGGCCA
GACGCGGTGGCTCACGCCTGTAATTCCAGCACTTTGGGAGGGCGAGGTGG
GCGGATCACCTGAGGTTGGGAGTTCGAGACCAGCCTGACCAACATGGAGA
AATCTGTCTCTACTAAAAATACAAAATTAGCCGATAATCCCAGCTACTCG
GGAGGCTGAGGCAGGAGAATCACTTGAACCCAGGAGGCAGAGGTTGCAGT
GAGCTGAGACTGAGCCACTGCACTCCAGCCTGGGCAACAAGAGGGAAACT
CTGTCTCAAAAAAACAAAACAAAACACAACTCTGCTGACAACCCATGCTG
TCCACACAGGCAGGGGTGGAAGCCCAATACGTGGCAGCTCAGATAGTGCA
CAGAAGCCAAGTAATAAATGTCTGCTTTCCTAGAGGAGATCTAAGGACCA
TCATGGCCACACTTGCCTATTTTTCCAGAGATGCTGGGAAATCCATTTTA
TTTTTTATTTTTATTTATTATTATTATTTTTTGAGACGGAGTCTTGCTCT
GTCGCCCAGGCCGGAGTGCATTGGGCGATCTTGGCTTAATGCAACCTCTG
CCTCCCGGGCTCAAGCGATTCTCCTGCCGCAGCCTCTGGAGTAGCTGGGA
TTACAGGCGCGCGACACCACGCCCGGCTAACTTTTCTTTATTTTCTTCTT
CTTCTTCTTCTTTTTTTTTTTTTTTTGTATTTTTTAGTAGATACTGGGTT
TCACCATGTTGGCCAGGTTAGTCTTGAACTCCGGACCTCAGGTGATCCAC
CTTCCTAGGCCTCCCAAAGTGCTGAGATTATGGGCATGAGCCACCGCGTC
CTGCCAGGAAATCCATTTTCTAAGTCCTAACTTTTAAGCACTGGCAACCA
ATCCCAAAGTTTCTTCAAACACAATGTGGGCCAAATAACACGTGTGGGAA
GTTCAAGCCTAAAGTACAAACTCCGGAGAGCAACACAAATATGGCTTGGA
CGTGGCCTGCCTCTTTCATCTCCCCTAATACATGCGGCGTACCAGCCACT
CTGAAGGGATCCCCTTCCGCCTTCCTGGCCTTTGCCCAGACGGTTCCCTC
CTCGTGAAACACCCTCTACCACTTCCTTGGCATCTTCAAGACCCTCATTA
AATACCATGTCCTCCCCTTGAGGAATCTTCCTCATCCCCACAGCCATTCT
TTGGGTTTCCTCCTTTCAAGCCGTGGCGTAACTGGGTCAGATATTTCTTT
GTACCCCCCAAAGGAAGAAAGGGGAACTAATTTGAAGTTCTGAAGACTTT
ATCTATGTTATTCCCATTTAATCCTCACACCAGGTCCGCAAAATGGGCGT
CACCTTTATCTTCTCCCTACAGAAAACAAAGCCACGTCTCAGAGAGGTTA
GGGGAATCCCCCAAGGTCACACAGCTCTGCGGAGTGGGGCAGAATCTGGA
ATAAAGGTCGGGTCAGTTTCCAAAAGCCAGGGCCCCTGCTGCTCTAGCTA
TTCTGCAGCTCTGAAAGTTTCACCTCGTTCCAGGAAAGCATTTCGTTCAA
AAAAATCCAGCCTTGAAAATATTAGGTGTGCGCGCGCCCGTGCGCGCAAA
CACACACACACAACACACACGCTATATACAGATACACACAGACACACACA
TTATGTGTATGTATATACTAGCAGATACCATCTATTAATATTACTGAAAC
AAGTTATCGCAGAGAAGTGACAAGCATGGAGACAGCCGCACACCCCTCGG
AACCGGTCCCCTCCACCAGCCTGCCCTGCCTTCGCCCGTGCCTTGACCCG
CAGTCGCACCGCAGGACAAAATGTCCCCTTTTCCTTCGGAAAGCAGAAGC
CAGTGTAAATGCAACCGGGAGAACCAGAGAAGGGAGTTGCAAGGCCGCTT
CCGCCGGGCCGCCCTGAGAAGGAGCGGGGTGAGCTAGTCCAGCGCGCGTA
CCCGGAGCGACCCCGGCGTGCGCGGCGCCTCCCTGCGCAGGCGTGCGGCG
TGCGGCCCGCGGCGTGCGGCCCGCGGCTGTTCCCGGCGCGGATACTTACT
GCGCGGCGGGGCGTGCAGGTCGCATCGATGTCGGTGCGCAGGCCACGGCG
GCCGCAGAGTGGGGCCAACTCTGCCGGCCGCGGGACCCGGGTGAGGGTCT
GGGCCTGGGCTGGGCTGGGTGACGCCAGGAGGCCGGCTACTGCGCGGCGC
CCGAGAGTCCACATGCTGCTCCGGGTCTGCCGCCCGCTCCGCCCTCCAGC
GCTGGGTGCTGCGGCGACCCCTGGTGGCCACTGGCCGCAGGCACTCTTCT
GTGGGGGAGCAGCTAGAGGTTAGACCTCAGGAAGAACTTCCCAGCTTAGC
ACTATTCGTGCATTTAACAAAAATGGAGAGCCTGCTTTGTGCAAAGCACG
GAGTGCAACCAGGACCCCTGACCCAAGGGAGACTGCAGCCTGGTGGCCCG
CCGCTTCTAAAATTCTAAACAGGAGGAACTTGGGAGCTGCTGTCTTGCTG
GGAAGTGGGTGCATGCACAAATTGAGGCTGCTTGGCCGCCGGTATGGGTT
TACAGCAGTTGGGTATGTGGGGCCAGGAGACGGATGCCTTGTGTAAATCT
GTACTCTGCCCACTTCCTAGCTGTGTGACCAGAGGGCAAGTTAATTAACC
TCTCTGTGCCTTGTTTATAAAATGCGTTTTATAAAAGTTCCTACTTCATA
GGATTGTGGTAAGGATAAAATGAGTTAAGCCCTGTAAAGTTTTTTTTGGA
CAGGGCATGTTCCTGTTAAGTGCACAATAAATATTGTTAATAGATTTTAT
TGATTGATTGATTGATTGAGACGGAGTCTCGCTCTGTCGCCCAGGCTGGA
GGGCAGTGGTGCGATCTCGGCTCACTGCAAGCTCTGCCTCCCGGTTTCAC
GCCATTCTCCTGCCTCAGCCTCCCGAGTAACTGGGACTACAGGCGCCCGC
CACCATGCCTGGCTAATTTTTTGTATTTTTTTTTTTTTTTAGTAGAGACG
GGTTTCACCGTGTTAGCCAGGATGGTCTCGATCTACTGACCTCGTGATCC
ACCCGCCTCATTTATTTGTTTGTTTGAGACAGAGTCTCACTCTGTTGCCC
AGGCTGGAGTGCAGTGGTGTGGTCCTGGCTTACTGCAACTTCTCCCTCCC
GGGCTCAAGCAGTCTTCCCATTTCAGCCTTCCAAGTAGCTGGGACTACAG
GTGTGCACCACCACACCCAGCTAGTTTTTGTATTTTTTGTAGAAAGGGGG
TTTCGCTATGTTGCCCAGGGTGGTCTTGAACTTCTAGGCTCAAACGATCT
GCCCGCCTGGGCCTCACAAAGTGCTGAGACGACAGGCGTGAGCCACCGTG
CCTTGCCTATTGTTGATAGATTTTAAAAGAGAATTTTATTTCTGTTACAT
ATTACAAAGGGATGCAAATTGTGAAAGTTTTCTTCACATTTTATATATGC
TTTAGAGGATGTCAAATGTGAGCCGAGTGTGGTAGTGCATGCCTGTGATC
TCAGCTACTCAGGAGGCTGAGGCGGGAGGATCACTTGAGCCTGGGAGCTC
AAGGCTGCAGTGAGCTATGATTGTGCCTGTGAATAGCCACAGCATTCCAG
CCCGGCCATCATAGCAAGATACTGTCTTAAAAAAAAAGAAAATGTCAGTT
GTGTACACTTTCCATGGCAGAGAAGATGGGAAGGTACTAAAGTTCTTCCA
AAGCCACTTTGTGAATCGTGAAGAGAGACATGTTTACATACTTAGGATGC
TAAATGACAAGTGTCAAGAGGAATGTAAACTAACAGGTGTGAGTGTTGAG
TGGGAGATTTATATAGAGCTAGGGATGGGGGAATCAAGGAAGTATTCATG
GAAGAAGTGGTGTTGGCACAGGGCCTTCAAAAATAGGGTGGAGGAGGTGG
GATCTGATTGCCTATCAAAGAGGTGGTCCAGGGCATTCTGGATGAGAGAC
ATCCTTCTGTGTTTAATTAATTCTAGGCAGCTGCTTAAATACTGTTATGT
ATATGCCAGGCCTGGAGAAATCATTTTCCCCCTGCAAAGTATCTGGAGAT
GCTTTTGATTGTCACAACAGGCAGCTGTGGGGTGGGTATGTTCTACTGGC
ATCTAGTAGGTAGAGGCCAGGGATGAAACATCCTGTGATGTGCAGGACAG
CCCCCCACCTGTGCAACAAAGAACTATCCAGTCCAAAATTCTGCCAAGAT
TGAGAAGCCCTCCTTGAAACCATAAAGGGAGAGCCCATAGGCATCAAAGG
GTAGTGTTTTCATTGCCAGCCATGGTCTCAGACTCTGCCACACCAACAGT
GAGGCCCTTCCTCACATCATCAGAACTGCATTATATACTCAGAGAGAGAA
AGCTGCATCATTAGTTATTATTATTATTATTATTTGAGACAGAGTCTTGC
TTTGTTGCCCAGGCTGGAATGCAGTGGTGCCATCTCTGCTCACTGCAATC
TCCACCTCCTGGGTTCAAGAGATTCTGTCTCAGCCTCCAGAGTAGCTGGG
ATTGCAGGTGCCCACCACCACGCCTGGCTAATTTTTGTATTTTTAGTAGA
GATGGGGTTTCATCATGTTGGCCAGGCTGGTCTCGAACTCCTGACCTCAG
GTGATCCGCCCGCCTCGGCCGCCCAAAGTGCTGAGATTACAGGCGTGAGC
CACCGCACCCTGTGCTACATCATTATTAGCTAGCATGCTTAGCAAGTTCT
GGCATAGAAAGGTCACTAGCAAATGCTCCAGCTCCCTGCAAAGTTGATAT
CATTGCCCCATCTTTTTGGCCAAAGAAATAGACATAGAATAGCATATCAG
AGAAGACGAAGGCTTCGCAAGAACTTGTCTTTCTCCACTTCCTTGGAGGT
AGTCAAGAGGAATCAGGCACACTTGGTAAGAAGCTGGGGCACCGTGGCGT
GGGCAAAGATGTTGAGGGACTCTGTGGTACACTGTAGCTGTTCCTCTCTT
GGTTGCTTTTAGCCTCAAGTAGGCAGATCCTAATAGCTGTCAGGTTATGG
GTAGGGGAGGATATCACCAGAATTCCCTTGCTCTGCCATTAGGATATTTT
GATAGGATTTATCAGAGGGCTATGATTATCAAATGTGGTCGCTGGGCCAG
CAGCCTCAGCATCACCTGGAAACTTGTTAGAAATACAAATTCTCAGGCCC
TATTCTCGATCTACTGAATCAGAATCTCTGGGGTTTCAAGGCCCAGCAAT
CCATGGTTTAACAAGACCTCCAGGTGATTCTAATGTGCTTTAAAGTTTCA
GAAGCACTGTGGTAGCATAGGCCTTGGGCAAAATTGTGAGTTAAGCCAGT
GTCACCAGGGTGATCATTTTAAATCTAAAAATCTAAATCTCAAATGTGAA
TCTAACCATAGCCCTCTGCTTTCACCACCATCAAGATAAATTCCAACTTT
CTTAGCATGACAGAGGAAGCTCTCTTGCCCACCTTAGCTCTCCCAGCGCC
CTTACCACTCTCTTCTGTACACCCCACTCTGCATATTGGGAGGCATCTAG
ACATCTTTGCTTCTTCAAGCTATTTGTAGTTCCTAGAACAAGTGATGCTT
TTTCCCTCTCTGGCTATCCCTCATGTTGTTCCTTAGTCTTCCATCCCTTT
GCCTGGTATGGAAGATTGGATCTTCCAAAGAAGGCCACACCAATATTTGT
CACATGTCATATGCTGTTTGGGACTTTGACATTCCTGTCACCCAGGGATG
TTATCTATTCTCCTCCCTTCGAATTCGGGTTTGGGCTACAACTATTCCAA
CCAACAGGGTACTGCAGAAGTGCTGCTTGGTGACTTCCAAGTCCAGGTCA
TAAAAATGATGCATCTTCCTTCTAACTCCCTCTCTCTCTCTTTTTTTTTT
T
>hg19_dna range = chr9:
71647062-71651966 strand = +repeat

(SEQ ID NO: 65)

AAAAAAAAAAAGAGAGAGAGAGGGAGTTAGAAGGAAGATGCATCATTTTT
ATGACCTGGACTTGGAAGTCACCAAGCAGCACTTCTGCAGTACCCTGTTG
GTTGGAATAGTTGTAGCCCAAACCCGAATTCGAAGGGAGGAGAATAGATA
ACATCCCTGGGTGACAGGAATGTCAAAGTCCCAAACAGCATATGACATGT
GACAAATATTGGTGTGGCCTTCTTTGGAAGATCCAATCTTCCATACCAGG
CAAAGGGATGGAAGACTAAGGAACAACATGAGGGATAGCCAGAGAGGGAA
AAAGCATCACTTGTTCTAGGAACTACAAATAGCTTGAAGAAGCAAAGATG
TCTAGATGCCTCCCAATATGCAGAGTGGGGTGTACAGAAGAGAGTGGTAA
GGGCGCTGGGAGAGCTAAGGTGGGCAAGAGAGCTTCCTCTGTCATGCTAA
GAAAGTTGGAATTTATCTTGATGGTGGTGAAAGCAGAGGGCTATGGTTAG
ATTCACATTTGAGATTTAGATTTTTAGATTTAAAATGATCACCCTGGTGA
CACTGGCTTAACTCACAATTTTGCCCAAGGCCTATGCTACCACAGTGCTT
CTGAAACTTTAAAGCACATTAGAATCACCTGGAGGTCTTGTTAAACCATG
GATTGCTGGGCCTTGAAACCCCAGAGATTCTGATTCAGTAGATCGAGAAT
AGGGCCTGAGAATTTGTATTTCTAACAAGTTTCCAGGTGATGCTGAGGCT
GCTGGCCCAGCGACCACATTTGATAATCATAGCCCTCTGATAAATCCTAT
CAAAATATCCTAATGGCAGAGCAAGGGAATTCTGGTGATATCCTCCCCTA
CCCATAACCTGACAGCTATTAGGATCTGCCTACTTGAGGCTAAAAGCAAC
CAAGAGAGGAACAGCTACAGTGTACCACAGAGTCCCTCAACATCTTTGCC
CACGCCACGGTGCCCCAGCTTCTTACCAAGTGTGCCTGATTCCTCTTGAC
TACCTCCAAGGAAGTGGAGAAAGACAAGTTCTTGCGAAGCCTTCGTCTTC
TCTGATATGCTATTCTATGTCTATTTCTTTGGCCAAAAAGATGGGGCAAT
GATATCAACTTTGCAGGGAGCTGGAGCATTTGCTAGTGACCTTTCTATGC
CAGAACTTGCTAAGCATGCTAGCTAATAATGATGTAGCACAGGGTGCGGT
GGCTCACGCCTGTAATCTCAGCACTTTGGGCGGCCGAGGCGGGCGGATCA
CCTGAGGTCAGGAGTTCGAGACCAGCCTGGCCAACATGATGAAACCCCAT
CTCTACTAAAAATACAAAAATTAGCCAGGCGTGGTGGTGGGCACCTGCAA
TCCCAGCTACTCTGGAGGCTGAGACAGAATCTCTTGAACCCAGGAGGTGG
AGATTGCAGTGAGCAGAGATGGCACCACTGCATTCCAGCCTGGGCAACAA
AGCAAGACTCTGTCTCAAATAATAATAATAATAATAACTAATGATGCAGC
TTTCTCTCTCTGAGTATATAATGCAGTTCTGATGATGTGAGGAAGGGCCT
CACTGTTGGTGTGGCAGAGTCTGAGACCATGGCTGGCAATGAAAACACTA
CCCTTTGATGCCTATGGGCTCTCCCTTTATGGTTTCAAGGAGGGCTTCTC
AATCTTGGCAGAATTTTGGACTGGATAGTTCTTTGTTGCACAGGTGGGGG
GCTGTCCTGCACATCACAGGATGTTTCATCCCTGGCCTCTACCTACTAGA
TGCCAGTAGAACATACCCACCCCACAGCTGCCTGTTGTGACAATCAAAAG
CATCTCCAGATACTTTGCAGGGGGAAAATGATTTCTCCAGGCCTGGCATA
TACATAACAGTATTTAAGCAGCTGCCTAGAATTAATTAAACACAGAAGGA
TGTCTCTCATCCAGAATGCCCTGGACCACCTCTTTGATAGGCAATCAGAT
CCCACCTCCTCCACCCTATTTTTGAAGGCCCTGTGCCAACACCACTTCTT
CCATGAATACTTCCTTGATTCCCCCATCCCTAGCTCTATATAAATCTCCC
ACTCAACACTCACACCTGTTAGTTTACATTCCTCTTGACACTTGTCATTT
AGCATCCTAAGTATGTAAACATGTCTCTCTTCACGATTCACAAAGTGGCT
TTGGAAGAACTTTAGTACCTTCCCATCTTCTCTGCCATGGAAAGTGTACA
CAACTGACATTTTCTTTTTTTTTAAGACAGTATCTTGCTATGATGGCCGG
GCTGGAATGCTGTGGCTATTCACAGGCACAATCATAGCTCACTGCAGCCT
TGAGCTCCCAGGCTCAAGTGATCCTCCCGCCTCAGCCTCCTGAGTAGCTG
AGATCACAGGCATGCACTACCACACTCGGCTCACATTTGACATCCTCTAA
AGCATATATAAAATGTGAAGAAAACTTTCACAATTTGCATCCCTTTGTAA
TATGTAACAGAAATAAAATTCTCTTTTAAAATCTATCAACAATAGGCAAG
GCACGGTGGCTCACGCCTGTCGTCTCAGCACTTTGTGAGGCCCAGGCGGG
CAGATCGTTTGAGCCTAGAAGTTCAAGACCACCCTGGGCAACATAGCGAA
ACCCCCTTTCTACAAAAAATACAAAAACTAGCTGGGTGTGGTGGTGCACA
CCTGTAGTCCCAGCTACTTGGAAGGCTGAAATGGGAAGACTGCTTGAGCC
CGGGAGGGAGAAGTTGCAGTAAGCCAGGACCACACCACTGCACTCCAGCC
TGGGCAACAGAGTGAGACTCTGTCTCAAACAAACAAATAAATGAGGCGGG
TGGATCACGAGGTCAGTAGATCGAGACCATCCTGGCTAACACGGTGAAAC
CCGTCTCTACTAAAAAAAAAAAAAAATACAAAAAATTAGCCAGGCATGGT
GGCGGGCGCCTGTAGTCCCAGTTACTCGGGAGGCTGAGGCAGGAGAATGG
CGTGAAACCGGGAGGCAGAGCTTGCAGTGAGCCGAGATCGCACCACTGCC
CTCCAGCCTGGGCGACAGAGCGAGACTCCGTCTCAATCAATCAATCAATC
AATAAAATCTATTAACAATATTTATTGTGCACTTAACAGGAACATGCCCT
GTCCAAAAAAAACTTTACAGGGCTTAACTCATTTTATCCTTACCACAATC
CTATGAAGTAGGAACTTTTATAAAACGCATTTTATAAACAAGGCACAGAG
AGGTTAATTAACTTGCCCTCTGGTCACACAGCTAGGAAGTGGGCAGAGTA
CAGATTTACACAAGGCATCCGTCTCCTGGCCCCACATACCCAACTGCTGT
AAACCCATACCGGCGGCCAAGCAGCCTCAATTTGTGCATGCACCCACTTC
CCAGCAAGACAGCAGCTCCCAAGTTCCTCCTGTTTAGAATTTTAGAAGCG
GCGGGCCACCAGGCTGCAGTCTCCCTTGGGTCAGGGGTCCTGGTTGCACT
CCGTGCTTTGCACAAAGCAGGCTCTCCATTTTTGTTAAATGCACGAATAG
TGCTAAGCTGGGAAGTTCTTCCTGAGGTCTAACCTCTAGCTGCTCCCCCA
CAGAAGAGTGCCTGCGGCCAGTGGCCACCAGGGGTCGCCGCAGCACCCAG
CGCTGGAGGGCGGAGCGGGCGGCAGACCCGGAGCAGCATGTGGACTCTCG
GGCGCCGCGCAGTAGCCGGCCTCCTGGCGTCACCCAGCCCAGCCCAGGCC
CAGACCCTCACCCGGGTCCCGCGGCCGGCAGAGTTGGCCCCACTCTGCGG
CCGCCGTGGCCTGCGCACCGACATCGATGCGACCTGCACGCCCCGCCGCG
CAGTAAGTATCCGCGCCGGGAACAGCCGCGGGCCGCACGCCGCGGGCCGC
ACGCCGCACGCCTGCGCAGGGAGGCGCCGCGCACGCCGGGGTCGCTCCGG
GTACGCGCGCTGGACTAGCTCACCCCGCTCCTTCTCAGGGCGGCCCGGCG
GAAGCGGCCTTGCAACTCCCTTCTCTGGTTCTCCCGGTTGCATTTACACT
GGCTTCTGCTTTCCGAAGGAAAAGGGGACATTTTGTCCTGCGGTGCGACT
GCGGGTCAAGGCACGGGCGAAGGCAGGGCAGGCTGGTGGAGGGGACCGGT
TCCGAGGGGTGTGCGGCTGTCTCCATGCTTGTCACTTCTCTGCGATAACT
TGTTTCAGTAATATTAATAGATGGTATCTGCTAGTATATACATACACATA
ATGTGTGTGTCTGTGTGTATCTGTATATAGCGTGTGTGTTGTGTGTGTGT
GTTTGCGCGCACGGGCGCGCGCACACCTAATATTTTCAAGGCTGGATTTT
TTTGAACGAAATGCTTTCCTGGAACGAGGTGAAACTTTCAGAGCTGCAGA
ATAGCTAGAGCAGCAGGGGCCCTGGCTTTTGGAAACTGACCCGACCTTTA
TTCCAGATTCTGCCCCACTCCGCAGAGCTGTGTGACCTTGGGGGATTCCC
CTAACCTCTCTGAGACGTGGCTTTGTTTTCTGTAGGGAGAAGATAAAGGT
GACGCCCATTTTGCGGACCTGGTGTGAGGATTAAATGGGAATAACATAGA
TAAAGTCTTCAGAACTTCAAATTAGTTCCCCTTTCTTCCTTTGGGGGGTA
CAAAGAAATATCTGACCCAGTTACGCCACGGCTTGAAAGGAGGAAACCCA
AAGAATGGCTGTGGGGATGAGGAAGATTCCTCAAGGGGAGGACATGGTAT
TTAATGAGGGTCTTGAAGATGCCAAGGAAGTGGTAGAGGGTGTTTCACGA
GGAGGGAACCGTCTGGGCAAAGGCCAGGAAGGCGGAAGGGGATCCCTTCA
GAGTGGCTGGTACGCCGCATGTATTAGGGGAGATGAAAGAGGCAGGCCAC
GTCCAAGCCATATTTGTGTTGCTCTCCGGAGTTTGTACTTTAGGCTTGAA
CTTCC
>hg19_dna range = chr9:
71647062-71651966 strand = −repeat

(SEQ ID NO: 66)

GGAAGTTCAAGCCTAAAGTACAAACTCCGGAGAGCAACACAAATATGGCT
TGGACGTGGCCTGCCTCTTTCATCTCCCCTAATACATGCGGCGTACCAGC
CACTCTGAAGGGATCCCCTTCCGCCTTCCTGGCCTTTGCCCAGACGGTTC
CCTCCTCGTGAAACACCCTCTACCACTTCCTTGGCATCTTCAAGACCCTC
ATTAAATACCATGTCCTCCCCTTGAGGAATCTTCCTCATCCCCACAGCCA
TTCTTTGGGTTTCCTCCTTTCAAGCCGTGGCGTAACTGGGTCAGATATTT
CTTTGTACCCCCCAAAGGAAGAAAGGGGAACTAATTTGAAGTTCTGAAGA
CTTTATCTATGTTATTCCCATTTAATCCTCACACCAGGTCCGCAAAATGG
GCGTCACCTTTATCTTCTCCCTACAGAAAACAAAGCCACGTCTCAGAGAG
GTTAGGGGAATCCCCCAAGGTCACACAGCTCTGCGGAGTGGGGCAGAATC
TGGAATAAAGGTCGGGTCAGTTTCCAAAAGCCAGGGCCCCTGCTGCTCTA
GCTATTCTGCAGCTCTGAAAGTTTCACCTCGTTCCAGGAAAGCATTTCGT
TCAAAAAAATCCAGCCTTGAAAATATTAGGTGTGCGCGCGCCCGTGCGCG
CAAACACACACACACAACACACACGCTATATACAGATACACACAGACACA
CACATTATGTGTATGTATATACTAGCAGATACCATCTATTAATATTACTG
AAACAAGTTATCGCAGAGAAGTGACAAGCATGGAGACAGCCGCACACCCC
TCGGAACCGGTCCCCTCCACCAGCCTGCCCTGCCTTCGCCCGTGCCTTGA
CCCGCAGTCGCACCGCAGGACAAAATGTCCCCTTTTCCTTCGGAAAGCAG
AAGCCAGTGTAAATGCAACCGGGAGAACCAGAGAAGGGAGTTGCAAGGCC
GCTTCCGCCGGGCCGCCCTGAGAAGGAGCGGGGTGAGCTAGTCCAGCGCG
CGTACCCGGAGCGACCCCGGCGTGCGCGGCGCCTCCCTGCGCAGGCGTGC
GGCGTGCGGCCCGCGGCGTGCGGCCCGCGGCTGTTCCCGGCGCGGATACT
TACTGCGCGGCGGGGCGTGCAGGTCGCATCGATGTCGGTGCGCAGGCCAC
GGCGGCCGCAGAGTGGGGCCAACTCTGCCGGCCGCGGGACCCGGGTGAGG
GTCTGGGCCTGGGCTGGGCTGGGTGACGCCAGGAGGCCGGCTACTGCGCG
GCGCCCGAGAGTCCACATGCTGCTCCGGGTCTGCCGCCCGCTCCGCCCTC
CAGCGCTGGGTGCTGCGGCGACCCCTGGTGGCCACTGGCCGCAGGCACTC
TTCTGTGGGGGAGCAGCTAGAGGTTAGACCTCAGGAAGAACTTCCCAGCT
TAGCACTATTCGTGCATTTAACAAAAATGGAGAGCCTGCTTTGTGCAAAG
CACGGAGTGCAACCAGGACCCCTGACCCAAGGGAGACTGCAGCCTGGTGG
CCCGCCGCTTCTAAAATTCTAAACAGGAGGAACTTGGGAGCTGCTGTCTT
GCTGGGAAGTGGGTGCATGCACAAATTGAGGCTGCTTGGCCGCCGGTATG
GGTTTACAGCAGTTGGGTATGTGGGGCCAGGAGACGGATGCCTTGTGTAA
ATCTGTACTCTGCCCACTTCCTAGCTGTGTGACCAGAGGGCAAGTTAATT
AACCTCTCTGTGCCTTGTTTATAAAATGCGTTTTATAAAAGTTCCTACTT
CATAGGATTGTGGTAAGGATAAAATGAGTTAAGCCCTGTAAAGTTTTTTT
TGGACAGGGCATGTTCCTGTTAAGTGCACAATAAATATTGTTAATAGATT
TTATTGATTGATTGATTGATTGAGACGGAGTCTCGCTCTGTCGCCCAGGC
TGGAGGGCAGTGGTGCGATCTCGGCTCACTGCAAGCTCTGCCTCCCGGTT
TCACGCCATTCTCCTGCCTCAGCCTCCCGAGTAACTGGGACTACAGGCGC
CCGCCACCATGCCTGGCTAATTTTTTGTATTTTTTTTTTTTTTTAGTAGA
GACGGGTTTCACCGTGTTAGCCAGGATGGTCTCGATCTACTGACCTCGTG
ATCCACCCGCCTCATTTATTTGTTTGTTTGAGACAGAGTCTCACTCTGTT
GCCCAGGCTGGAGTGCAGTGGTGTGGTCCTGGCTTACTGCAACTTCTCCC
TCCCGGGCTCAAGCAGTCTTCCCATTTCAGCCTTCCAAGTAGCTGGGACT
ACAGGTGTGCACCACCACACCCAGCTAGTTTTTGTATTTTTTGTAGAAAG
GGGGTTTCGCTATGTTGCCCAGGGTGGTCTTGAACTTCTAGGCTCAAACG
ATCTGCCCGCCTGGGCCTCACAAAGTGCTGAGACGACAGGCGTGAGCCAC
CGTGCCTTGCCTATTGTTGATAGATTTTAAAAGAGAATTTTATTTCTGTT
ACATATTACAAAGGGATGCAAATTGTGAAAGTTTTCTTCACATTTTATAT
ATGCTTTAGAGGATGTCAAATGTGAGCCGAGTGTGGTAGTGCATGCCTGT
GATCTCAGCTACTCAGGAGGCTGAGGCGGGAGGATCACTTGAGCCTGGGA
GCTCAAGGCTGCAGTGAGCTATGATTGTGCCTGTGAATAGCCACAGCATT
CCAGCCCGGCCATCATAGCAAGATACTGTCTTAAAAAAAAAGAAAATGTC
AGTTGTGTACACTTTCCATGGCAGAGAAGATGGGAAGGTACTAAAGTTCT
TCCAAAGCCACTTTGTGAATCGTGAAGAGAGACATGTTTACATACTTAGG
ATGCTAAATGACAAGTGTCAAGAGGAATGTAAACTAACAGGTGTGAGTGT
TGAGTGGGAGATTTATATAGAGCTAGGGATGGGGGAATCAAGGAAGTATT
CATGGAAGAAGTGGTGTTGGCACAGGGCCTTCAAAAATAGGGTGGAGGAG
GTGGGATCTGATTGCCTATCAAAGAGGTGGTCCAGGGCATTCTGGATGAG
AGACATCCTTCTGTGTTTAATTAATTCTAGGCAGCTGCTTAAATACTGTT
ATGTATATGCCAGGCCTGGAGAAATCATTTTCCCCCTGCAAAGTATCTGG
AGATGCTTTTGATTGTCACAACAGGCAGCTGTGGGGTGGGTATGTTCTAC
TGGCATCTAGTAGGTAGAGGCCAGGGATGAAACATCCTGTGATGTGCAGG
ACAGCCCCCCACCTGTGCAACAAAGAACTATCCAGTCCAAAATTCTGCCA
AGATTGAGAAGCCCTCCTTGAAACCATAAAGGGAGAGCCCATAGGCATCA
AAGGGTAGTGTTTTCATTGCCAGCCATGGTCTCAGACTCTGCCACACCAA
CAGTGAGGCCCTTCCTCACATCATCAGAACTGCATTATATACTCAGAGAG
AGAAAGCTGCATCATTAGTTATTATTATTATTATTATTTGAGACAGAGTC
TTGCTTTGTTGCCCAGGCTGGAATGCAGTGGTGCCATCTCTGCTCACTGC
AATCTCCACCTCCTGGGTTCAAGAGATTCTGTCTCAGCCTCCAGAGTAGC
TGGGATTGCAGGTGCCCACCACCACGCCTGGCTAATTTTTGTATTTTTAG
TAGAGATGGGGTTTCATCATGTTGGCCAGGCTGGTCTCGAACTCCTGACC
TCAGGTGATCCGCCCGCCTCGGCCGCCCAAAGTGCTGAGATTACAGGCGT
GAGCCACCGCACCCTGTGCTACATCATTATTAGCTAGCATGCTTAGCAAG
TTCTGGCATAGAAAGGTCACTAGCAAATGCTCCAGCTCCCTGCAAAGTTG
ATATCATTGCCCCATCTTTTTGGCCAAAGAAATAGACATAGAATAGCATA
TCAGAGAAGACGAAGGCTTCGCAAGAACTTGTCTTTCTCCACTTCCTTGG
AGGTAGTCAAGAGGAATCAGGCACACTTGGTAAGAAGCTGGGGCACCGTG
GCGTGGGCAAAGATGTTGAGGGACTCTGTGGTACACTGTAGCTGTTCCTC
TCTTGGTTGCTTTTAGCCTCAAGTAGGCAGATCCTAATAGCTGTCAGGTT
ATGGGTAGGGGAGGATATCACCAGAATTCCCTTGCTCTGCCATTAGGATA
TTTTGATAGGATTTATCAGAGGGCTATGATTATCAAATGTGGTCGCTGGG
CCAGCAGCCTCAGCATCACCTGGAAACTTGTTAGAAATACAAATTCTCAG
GCCCTATTCTCGATCTACTGAATCAGAATCTCTGGGGTTTCAAGGCCCAG
CAATCCATGGTTTAACAAGACCTCCAGGTGATTCTAATGTGCTTTAAAGT
TTCAGAAGCACTGTGGTAGCATAGGCCTTGGGCAAAATTGTGAGTTAAGC
CAGTGTCACCAGGGTGATCATTTTAAATCTAAAAATCTAAATCTCAAATG
TGAATCTAACCATAGCCCTCTGCTTTCACCACCATCAAGATAAATTCCAA
CTTTCTTAGCATGACAGAGGAAGCTCTCTTGCCCACCTTAGCTCTCCCAG
CGCCCTTACCACTCTCTTCTGTACACCCCACTCTGCATATTGGGAGGCAT
CTAGACATCTTTGCTTCTTCAAGCTATTTGTAGTTCCTAGAACAAGTGAT
GCTTTTTCCCTCTCTGGCTATCCCTCATGTTGTTCCTTAGTCTTCCATCC
CTTTGCCTGGTATGGAAGATTGGATCTTCCAAAGAAGGCCACACCAATAT
TTGTCACATGTCATATGCTGTTTGGGACTTTGACATTCCTGTCACCCAGG
GATGTTATCTATTCTCCTCCCTTCGAATTCGGGTTTGGGCTACAACTATT
CCAACCAACAGGGTACTGCAGAAGTGCTGCTTGGTGACTTCCAAGTCCAG
GTCATAAAAATGATGCATCTTCCTTCTAACTCCCTCTCTCTCTCTTTTTT
TTTTT
>hg19_dna range = chr9:
71652468-71657262 strand = +repeat

(SEQ ID NO: 67)

CTTAGATCTCCTCTAGGAAAGCAGACATTTATTACTTGGCTTCTGTGCAC
TATCTGAGCTGCCACGTATTGGGCTTCCACCCCTGCCTGTGTGGACAGCA
TGGGTTGTCAGCAGAGTTGTGTTTTGTTTTGTTTTTTTGAGACAGAGTTT
CCCTCTTGTTGCCCAGGCTGGAGTGCAGTGGCTCAGTCTCAGCTCACTGC
AACCTCTGCCTCCTGGGTTCAAGTGATTCTCCTGCCTCAGCCTCCCGAGT
AGCTGGGATTATCGGCTAATTTTGTATTTTTAGTAGAGACAGATTTCTCC
ATGTTGGTCAGGCTGGTCTCGAACTCCCAACCTCAGGTGATCCGCCCACC
TCGCCCTCCCAAAGTGCTGGAATTACAGGCGTGAGCCACCGCGTCTGGCC
ATCAGCAGAGTTTTTAATTTAGGAGAATGACAAGAGGTGGTACAGTTTTT
TAGATGGTACCTGGTGGCTGTTAAGGGCTATTGACTGACAAACACACCCA
ACTTGGCGCTGCCGCCCAGGAGGTGGACACTGGGTTTCTGGATAGATGGT
TAGCAACCTCTGTCACCAGCTGGGCCTCTTTTTTTCTATACTGAATTAAT
CACATTTGTTTAACCTGTCTGTTCCATAGTTCCCTTGCACATCTTGGGTA
TTTGAGGAGTTGGGTGGGTGGCAGTGGCAACTGGGGCCACCATCCTGTTT
AATTATTTTAAAGCCCTGACTGTCCTGGATTGACCCTAAGCTCCCCCTGG
TCTCCAAAATTCATCAGAAACTGAGTTCACTTGAAGGCCTCTTCCCCACC
CTTTTCTCCACCCCTTGCATCTACTTCTAAAGCAGCTGTTCAACAGAAAC
AGAATGGGAGCCACACACATAATTCTACATTTTCTAGTTAAAAAGAAAAA
AAAATCATTTTCAACAATATATTTATTCAACCTAGTACATACAAAATATT
ATCATTCCAACATGTAATCAGTATTTTAAAAATCAGTAATGAGACCAGGC
ACGGTGGCTCACGACTGTAATCCCAGGACTTTGGGAGGCCGAGGCGAGTG
GATCATCTGAGATCAGGAGTTCAAGACCAGCCTGGCCAACATGGTGAAAC
CCCATCTCTACTAAAAACTAGCTCAGCATGGTGGTGGGTGCCTGTAGTCC
CAGCTACTCGGGAGGCTGAGGCATGAGAATCACTTGAGCCCAGGAGGCAG
AGGTTGCAGTGAGCCAAGATTTTGGGGGATTCTGTGACATACAAAAAAAA
TCAGTAATAAGATATCTTGCATACTCTTTTCGTACTCATATACTTCCAGC
ATATCTCAATTCACAATTTCTAAGTAAATGCTCTATCTGTATTTACTTTT
ATAAAATTCACAATTAAAAATGAAGGTTCACATAGTCAAGTTGTTCCAAA
CACACTTAAATGTCTCCTAGGCTGGGTGTGGTTGCTCACACCTGTAATCC
CAGCACTTTGGGAGGCTGAGATGGGCGGATCACCTGAGGTCAGGAGTTTG
AGACCAGCCTGGCCAACATGGTGAAACCCCGTCTCTACTAAAAATACAAA
AATTAGCTGGATGTGGTGGCACTCACCTGTAATCCCAGCTACTCAGGAGG
CTGAGGCAGGATAATTGCTTGAACCCGGGAGGTGGTGGAGGTTGCAGTGA
GCCGAGATCGCACCACTGCCTTCCAACCTGGGCGACAGAGCGAGACTCCG
TCTCAAAAAAAAAAAAAAGGCTCCTAATAACTTTATTACTTTATTATCAC
CTCAAATAATTAAAATTAAATGAAGTTGAAAATCCAGGTCCTCAGTCCCA
TTAGCCACATTTCTAGTGCTCAGTAGCCACGGGGGCTGGTGACCACCACA
TGGGACAGCATATTTAGTACCTGATCATTGGTTCTCAGATCTGGCTACTC
AGCAGAACCAAGAATCCACAGAAACGGCTTTTAAAAGCACAGCCCCACAG
CCCCCAGCCCCAGCCTTACCTACCTGGAGGCTGGGAAGGACTCTGATTCC
ACGAGGCAGCCTATGTTTTTTGATGGAGGGATGTGACAGGGGCTGCATCT
TTAACGTTTCCTCTTAAATACTGGAGACAGCTTCGAGGAGGAGATAACTG
GATGTGTCTTAGTCCATTTGATGGAGGGATGTGACGGGGCTGCGTCTTTA
ACGTTTCCTCTTAAATACCGGAGACAGCTTCGAGAAGGAGATAACTGGAT
GTTTCTTAGTCCATTTTCTGTTGCTTGTGACAGAATACCTGAAACTGGGC
AATTTATATGGTAAAAAATTTTCTTCTTACTGCTCTGGAGGCTGAGAAGT
CCAAAGTCAAGTCCCTTCTTGCTGGTGGGGACTTTGCAGAGTATTGAGGC
GGCACCGGGCGTCATATGGTAAGGGGCTGAGTGTGCTACCTCAGGTGTCT
TTTTCTTTTCTTATAAAGCCTAACTAGTTTCACTCCCATGATAACCCATT
AATCTATGAATGGATTAATCCATTATTGAGGGAAGAACCTTCATGACCCA
GTCACCGCTTAAAGGCCCCACCTCTCAATACTGCCACATCGGGAATTAAG
TTTCAACATGAGTTTCGGAGGTGACAAACATTCAAACCATAGCATGCTGT
CTCTTAAATGACTCAATAAGCTCCTGTGGCATCCACTTCTGCATGCCTTG
GGCAGCTTTTAGACATCTGTCCATTTTCCTAGAGGGACAAGACCACCACC
TGTGATCCTATGACCTTTTGGCTTTAGGCCTAACAAGCAGGTTATACCCT
CACTCACTTTCAAATCATTTTTATTGTCTTGCAGACAATTTACACAAGTT
TACACATAGAAAAGGATATGTAAATATTTATACGCTGCCGGGCGCGGTGG
CTCACGCCTGTAATCCCAGCACTTTGGGAGGCCGAGGCAGGTGGATCACG
AGTTCAGGAGATGGAGACCATCCTGGCTAATACGATGAAACCCCATCTCT
ACTAAAAATACAAAAAATTAGCCGGGCGTGGTGACGGGTGCCTGTAGTCC
CCACTACTCGGGACGCTGAGGCAGGAGAATGGCGTGAACCCGGGAGGCAG
AGCTTGCAGTGATCCGAGATCGTGCCACTGCACTCCAGCCTGGGTGACAG
AGCGAGACTGCATCTCAAAGAAAAAAATAAATAAATAAATAAATATTTAT
ACTGCTTATAAACTAATAATAAATGCTATGGTCTGCATGTTTGTGTCACC
CCACCATTCATATGTTAAAACCTAATCACCAAAGTGATATTAGGAGGTGG
GGCCCTTGGGAGGTGATGAGGTATGAGGGTGGAGCCCATATGATTGGGAT
TAGTGCCCTTCTAAAATAGCCCAACGGAGCCCAGTGACAAGGCATCATCT
ATGAACCAGGAAACTGGCCCTCACCAGACACCAAAGCTGTTGGTGCATTG
ATCTTGGATTTCCCACCCTCCAGGACTCTAAGAAACACATTTCTATTGTT
TATAAGCCACCCAGTGGCTGGTATTTTGTTATAACATCCCAGACTAAGAC
AAATAACAAATACTTGTATCCCTGACACCAGGTTAAGAGATAGAATTTGT
TTGTTCCTCTGGAGGCCCTTGTCTTCACCCCATCACTGCCCTGTCCTCCC
TGGAGGAATCTGCCAGCCCGAATTCTGTTCATCGTACCCTCCTTTTCTTA
GAGTTTGACCTCCTCTGTATCTCCCCCAATCCATGTATTGCTTATATACA
AGGTATTCTGCTGTATCTGTTCTGCTATGGCTTGCCCCTTTTGTTCAACA
CTGTTTTTGTGCGTCATCTGCATTGATGCATGCAGTTGTCCTTTATTTGT
TCTCACTGCTGGATAGTATCTGGTTGGGTAAATATATCACACTGTAAATC
ACACTATCCAGGTTCCTTTAGGTGACATTTGGTTGATTGCAGTGTTCTGT
TGTTACGATGGTGCTGCTGTGACTGTTCTTGTGCATGGACAGAAGTTCCT
TTCAGGTGAATTTCTCAGAATGGAATTGCTGGGCAAAGGGGCAGCCAATA
ATCAACTCATTTGATGCCAAAAGTGGTGGTGCCAGTTCATCCTCCCCTGC
GAGGTATGGGTCCTGATTCACTCTTCAAGTGCTGTGGTTTGACAGGGCCG
GGGGTGACAAGGGGACACCTGGGAAGGAAAGCTGGGCTCCCTGCTGGCCA
TCCAGGCCAGTCCTTACCAGGGGGTAGGCAATGATTGGGTCAAGTGGTTC
CTGACCACTGGGCCTGAGACTTCAGGCCCAGAAACTATCTAATATTTCCT
CAAATGCATCCCATGAGCAGGCACTGTGTGAGTGAGCACACACATCTGAA
GCCTCAAGCTAGGCAAGCCTACCATGACTTGTGGTCCAAGGGCTCACGGG
TGACCTGGAGTTAGAGGGAGACATGGCTGCCAGGTGGCTTTAGAAAGAAC
ACTCATCATGGCCAGGTGCGGTGGCTTACGCCTGTAATCCCAGCACTTTG
GGAGGCCAAGGTGGGTGGATCATGAGGTCAGGAGTGAGACCAGCCTGACC
AACATGCTGAAACCTGTCTCTCCTAAAAACACAAAAATTAGCTGGGCATG
GAGGTGCACGCCTGTAATCCCAGCTACTCAGGAGGCTGAGGCAGGAGAAT
CACTTGAACCCGGGAGGCGGAGGTTGCAATAAGCCTAGATTGTGCCACTG
CATTCCAGCCTGGGCAACAGAGCAAGACTCCGTCTCAGAAAAAAAAAAAA
AAAGGAAGAACACTCATCCTATGACCTTGACCTCCAAGCTTTGCCTCCCT
CAAGCAGAACAGAATGGAGCCTCCCTTAGGCAGAGGCGGAAGTTT
>hg19_dna range = chr9:
71652468-71657262 strand = −repeat

(SEQ ID NO: 68)

AAACTTCCGCCTCTGCCTAAGGGAGGCTCCATTCTGTTCTGCTTGAGGGA
GGCAAAGCTTGGAGGTCAAGGTCATAGGATGAGTGTTCTTCCTTTTTTTT
TTTTTTTCTGAGACGGAGTCTTGCTCTGTTGCCCAGGCTGGAATGCAGTG
GCACAATCTAGGCTTATTGCAACCTCCGCCTCCCGGGTTCAAGTGATTCT
CCTGCCTCAGCCTCCTGAGTAGCTGGGATTACAGGCGTGCACCTCCATGC
CCAGCTAATTTTTGTGTTTTTAGGAGAGACAGGTTTCAGCATGTTGGTCA
GGCTGGTCTCACTCCTGACCTCATGATCCACCCACCTTGGCCTCCCAAAG
TGCTGGGATTACAGGCGTAAGCCACCGCACCTGGCCATGATGAGTGTTCT
TTCTAAAGCCACCTGGCAGCCATGTCTCCCTCTAACTCCAGGTCACCCGT
GAGCCCTTGGACCACAAGTCATGGTAGGCTTGCCTAGCTTGAGGCTTCAG
ATGTGTGTGCTCACTCACACAGTGCCTGCTCATGGGATGCATTTGAGGAA
ATATTAGATAGTTTCTGGGCCTGAAGTCTCAGGCCCAGTGGTCAGGAACC
ACTTGACCCAATCATTGCCTACCCCCTGGTAAGGACTGGCCTGGATGGCC
AGCAGGGAGCCCAGCTTTCCTTCCCAGGTGTCCCCTTGTCACCCCCGGCC
CTGTCAAACCACAGCACTTGAAGAGTGAATCAGGACCCATACCTCGCAGG
GGAGGATGAACTGGCACCACCACTTTTGGCATCAAATGAGTTGATTATTG
GCTGCCCCTTTGCCCAGCAATTCCATTCTGAGAAATTCACCTGAAAGGAA
CTTCTGTCCATGCACAAGAACAGTCACAGCAGCACCATCGTAACAACAGA
ACACTGCAATCAACCAAATGTCACCTAAAGGAACCTGGATAGTGTGATTT
ACAGTGTGATATATTTACCCAACCAGATACTATCCAGCAGTGAGAACAAA
TAAAGGACAACTGCATGCATCAATGCAGATGACGCACAAAAACAGTGTTG
AACAAAAGGGGCAAGCCATAGCAGAACAGATACAGCAGAATACCTTGTAT
ATAAGCAATACATGGATTGGGGGAGATACAGAGGAGGTCAAACTCTAAGA
AAAGGAGGGTACGATGAACAGAATTCGGGCTGGCAGATTCCTCCAGGGAG
GACAGGGCAGTGATGGGGTGAAGACAAGGGCCTCCAGAGGAACAAACAAA
TTCTATCTCTTAACCTGGTGTCAGGGATACAAGTATTTGTTATTTGTCTT
AGTCTGGGATGTTATAACAAAATACCAGCCACTGGGTGGCTTATAAACAA
TAGAAATGTGTTTCTTAGAGTCCTGGAGGGTGGGAAATCCAAGATCAATG
CACCAACAGCTTTGGTGTCTGGTGAGGGCCAGTTTCCTGGTTCATAGATG
ATGCCTTGTCACTGGGCTCCGTTGGGCTATTTTAGAAGGGCACTAATCCC
AATCATATGGGCTCCACCCTCATACCTCATCACCTCCCAAGGGCCCCACC
TCCTAATATCACTTTGGTGATTAGGTTTTAACATATGAATGGTGGGGTGA
CACAAACATGCAGACCATAGCATTTATTATTAGTTTATAAGCAGTATAAA
TATTTATTTATTTATTTATTTTTTTCTTTGAGATGCAGTCTCGCTCTGTC
ACCCAGGCTGGAGTGCAGTGGCACGATCTCGGATCACTGCAAGCTCTGCC
TCCCGGGTTCACGCCATTCTCCTGCCTCAGCGTCCCGAGTAGTGGGGACT
ACAGGCACCCGTCACCACGCCCGGCTAATTTTTTGTATTTTTAGTAGAGA
TGGGGTTTCATCGTATTAGCCAGGATGGTCTCCATCTCCTGAACTCGTGA
TCCACCTGCCTCGGCCTCCCAAAGTGCTGGGATTACAGGCGTGAGCCACC
GCGCCCGGCAGCGTATAAATATTTACATATCCTTTTCTATGTGTAAACTT
GTGTAAATTGTCTGCAAGACAATAAAAATGATTTGAAAGTGAGTGAGGGT
ATAACCTGCTTGTTAGGCCTAAAGCCAAAAGGTCATAGGATCACAGGTGG
TGGTCTTGTCCCTCTAGGAAAATGGACAGATGTCTAAAAGCTGCCCAAGG
CATGCAGAAGTGGATGCCACAGGAGCTTATTGAGTCATTTAAGAGACAGC
ATGCTATGGTTTGAATGTTTGTCACCTCCGAAACTCATGTTGAAACTTAA
TTCCCGATGTGGCAGTATTGAGAGGTGGGGCCTTTAAGCGGTGACTGGGT
CATGAAGGTTCTTCCCTCAATAATGGATTAATCCATTCATAGATTAATGG
GTTATCATGGGAGTGAAACTAGTTAGGCTTTATAAGAAAAGAAAAAGACA
CCTGAGGTAGCACACTCAGCCCCTTACCATATGACGCCCGGTGCCGCCTC
AATACTCTGCAAAGTCCCCACCAGCAAGAAGGGACTTGACTTTGGACTTC
TCAGCCTCCAGAGCAGTAAGAAGAAAATTTTTTACCATATAAATTGCCCA
GTTTCAGGTATTCTGTCACAAGCAACAGAAAATGGACTAAGAAACATCCA
GTTATCTCCTTCTCGAAGCTGTCTCCGGTATTTAAGAGGAAACGTTAAAG
ACGCAGCCCCGTCACATCCCTCCATCAAATGGACTAAGACACATCCAGTT
ATCTCCTCCTCGAAGCTGTCTCCAGTATTTAAGAGGAAACGTTAAAGATG
CAGCCCCTGTCACATCCCTCCATCAAAAAACATAGGCTGCCTCGTGGAAT
CAGAGTCCTTCCCAGCCTCCAGGTAGGTAAGGCTGGGGCTGGGGGCTGTG
GGGCTGTGCTTTTAAAAGCCGTTTCTGTGGATTCTTGGTTCTGCTGAGTA
GCCAGATCTGAGAACCAATGATCAGGTACTAAATATGCTGTCCCATGTGG
TGGTCACCAGCCCCCGTGGCTACTGAGCACTAGAAATGTGGCTAATGGGA
CTGAGGACCTGGATTTTCAACTTCATTTAATTTTAATTATTTGAGGTGAT
AATAAAGTAATAAAGTTATTAGGAGCCTTTTTTTTTTTTTTGAGACGGAG
TCTCGCTCTGTCGCCCAGGTTGGAAGGCAGTGGTGCGATCTCGGCTCACT
GCAACCTCCACCACCTCCCGGGTTCAAGCAATTATCCTGCCTCAGCCTCC
TGAGTAGCTGGGATTACAGGTGAGTGCCACCACATCCAGCTAATTTTTGT
ATTTTTAGTAGAGACGGGGTTTCACCATGTTGGCCAGGCTGGTCTCAAAC
TCCTGACCTCAGGTGATCCGCCCATCTCAGCCTCCCAAAGTGCTGGGATT
ACAGGTGTGAGCAACCACACCCAGCCTAGGAGACATTTAAGTGTGTTTGG
AACAACTTGACTATGTGAACCTTCATTTTTAATTGTGAATTTTATAAAAG
TAAATACAGATAGAGCATTTACTTAGAAATTGTGAATTGAGATATGCTGG
AAGTATATGAGTACGAAAAGAGTATGCAAGATATCTTATTACTGATTTTT
TTTGTATGTCACAGAATCCCCCAAAATCTTGGCTCACTGCAACCTCTGCC
TCCTGGGCTCAAGTGATTCTCATGCCTCAGCCTCCCGAGTAGCTGGGACT
ACAGGCACCCACCACCATGCTGAGCTAGTTTTTAGTAGAGATGGGGTTTC
ACCATGTTGGCCAGGCTGGTCTTGAACTCCTGATCTCAGATGATCCACTC
GCCTCGGCCTCCCAAAGTCCTGGGATTACAGTCGTGAGCCACCGTGCCTG
GTCTCATTACTGATTTTTAAAATACTGATTACATGTTGGAATGATAATAT
TTTGTATGTACTAGGTTGAATAAATATATTGTTGAAAATGATTTTTTTTT
CTTTTTAACTAGAAAATGTAGAATTATGTGTGTGGCTCCCATTCTGTTTC
TGTTGAACAGCTGCTTTAGAAGTAGATGCAAGGGGTGGAGAAAAGGGTGG
GGAAGAGGCCTTCAAGTGAACTCAGTTTCTGATGAATTTTGGAGACCAGG
GGGAGCTTAGGGTCAATCCAGGACAGTCAGGGCTTTAAAATAATTAAACA
GGATGGTGGCCCCAGTTGCCACTGCCACCCACCCAACTCCTCAAATACCC
AAGATGTGCAAGGGAACTATGGAACAGACAGGTTAAACAAATGTGATTAA
TTCAGTATAGAAAAAAAGAGGCCCAGCTGGTGACAGAGGTTGCTAACCAT
CTATCCAGAAACCCAGTGTCCACCTCCTGGGCGGCAGCGCCAAGTTGGGT
GTGTTTGTCAGTCAATAGCCCTTAACAGCCACCAGGTACCATCTAAAAAA
CTGTACCACCTCTTGTCATTCTCCTAAATTAAAAACTCTGCTGATGGCCA
GACGCGGTGGCTCACGCCTGTAATTCCAGCACTTTGGGAGGGCGAGGTGG
GCGGATCACCTGAGGTTGGGAGTTCGAGACCAGCCTGACCAACATGGAGA
AATCTGTCTCTACTAAAAATACAAAATTAGCCGATAATCCCAGCTACTCG
GGAGGCTGAGGCAGGAGAATCACTTGAACCCAGGAGGCAGAGGTTGCAGT
GAGCTGAGACTGAGCCACTGCACTCCAGCCTGGGCAACAAGAGGGAAACT
CTGTCTCAAAAAAACAAAACAAAACACAACTCTGCTGACAACCCATGCTG
TCCACACAGGCAGGGGTGGAAGCCCAATACGTGGCAGCTCAGATAGTGCA
CAGAAGCCAAGTAATAAATGTCTGCTTTCCTAGAGGAGATCTAAG

In some embodiments, an oligonucleotide comprises a sequence represented by the formula (X₁X₂X₃)ₙ, in which X is any nucleotide, and in which n is 4-20. In some embodiments, an oligonucleotide comprises a sequence represented by the formula (X₁X₂X₃X₄)ₙ, in which X is any nucleotide, and in which n is 4-20. In some embodiments, X₁X₂X₃X₄ is CCCC or GGGG. In some embodiments, an oligonucleotide comprises a sequence represented by the formula (X₁X₂X₃X₄X₅)ₙ, in which X is any nucleotide, and in which n is 4-20. In some embodiments, X₁X₂X₃X₄X₅ is ATTCT or AGAAT. In some embodiments, the oligonucleotide includes non-repeat sequences on one or both sides of the repeat sequence that are complementary to sequences adjacent to the repeat region in its genomic context.

Any gene that is regulated by a heterochromatin forming non-coding RNA may be targeted using the oligonucleotides and methods disclosed herein. In some embodiments, the target gene is selected from the group consisting of: DMPK, CNBP, CSTB, FMR1, AFF2/FMR3, DIP2B, FXN, ATXN10, ATXN8/ATXN8OS, JPH3, and PPP2R2B. Further information regarding these genes and their associated diseases is provided in Table 1 below.

TABLE 1
Repeat expansion genes and related diseases

| | | | | Normal | | |
|---|---|---|---|---|---|---|
| | Affected | | Repeat | Repeat | Symptomatic | OMIM |
| Disorder | Gene | Repeat | Location | No. | Repeat No. | No. |
| Myotonic | DMPL | CTG | 3′ UTR | 5-37 | >50->2000 | 160900 |
| dystrophy type 1 | | | | | | |
| Myotonic | CNBP | CCTG | Intron 1 | <27 | 75-11000 | 608768 |
| dystrophy type 2 | | | | | | |
| progressive | CSTB | (C)₄G(C)₄GCG | Promoter | 2-3 | 30-75 | 254800 |
| myoclonus | | | | | | |
| epilepsy type I | | | | | | |
| Fragile X | FMR1 | CGG | 5′ UTR | 6-52 | ~55->2000 | 309550 |
| syndrome | | | | | | |
| (FRAXE) Mental | AFF2/FMR3 | CCG | 5′ end | 6-25 | >200 | 309548 |
| Retardation | | | | | | |
| (FRA12A) | DIP2B | CGG | 5′ UTR | 6-23 | | 136630 |
| Mental | | | | | | |
| Retardation | | | | | | |
| Freidreich's | FXN | GAA | Intron 1 | 7-22 | >66->900 | 229300 |
| ataxia | | | | | | |
| (SCA10) | ATXN10 | ATTCT | Intron 9 | 10-29 | 280-4500 | 603516 |
| spinocerebellar | | | | | | |
| ataxia | | | | | | |
| (SCA8) | ATXN8OS | CTG | Non- | 6-37 | ~107-250 | 603680 |
| spinocerebellar | | | coding | | | |
| ataxia | | | transcript | | | |
| (HDL-2) | JPH3 | CAG/CTG | | <50 | >50 | 606438 |
| Huntington | | | | | | |
| disease-like 2 | | | | | | |
| (SCA12) | PPP2R2B | CAG/CTG | | <66 | >66 | 604326 |
| spinocerebellar | | | | | | |
| ataxia | | | | | | |

In some embodiments, the target gene is FXN. In a small percentage of Freidreich's ataxia patients the GAA repeat is not pure (e.g., may contain GGA or other similar sequences). Accordingly, in some embodiments, the oligonucleotide sequence may be adjusted to target impure GAA repeats (e.g., by incorporating GGA or other similar sequences into the oligonucleotide).

### Oligonucleotides

In some embodiments, methods are provided for producing candidate oligonucleotides that are useful for eliminating or reversing heterochromatin at a gene and thereby activating or inducing expression the gene. Generally, the oligonucleotides are complementary to sequences in a genomic region encoding a heterochromatin forming non-coding RNA that regulates expression of the gene.

Typically, the oligonucleotides are designed by determining a genomic location of a target gene within which is expressed a heterochromatin forming non-coding RNA that regulates the target gene; producing an oligonucleotide that has a region of complementarity that is complementary with a plurality of (e.g., at least 5) contiguous nucleotides of the heterochromatin forming non-coding RNA or a reverse complementary sequence thereof; and determining whether administering the oligonucleotide to a cell in which the gene is silenced or downregulated due to heterochromatin formation results in induction of expression of the gene and/or reduction or elimination of the heterochromatin at the gene.

In some embodiments, methods are provided for obtaining one or more oligonucleotides for increasing expression of a target gene that further involve producing a plurality of different oligonucleotides, in which each oligonucleotide has a region of complementarity that is complementary with a plurality of (e.g., at least 5) contiguous nucleotides in a heterochromatin forming RNA or complement thereof; subjecting each of the different oligonucleotides to an assay that assesses whether delivery of an oligonucleotide to a cell harboring the target gene results in increased expression of the target gene in the cell; and obtaining one or more oligonucleotides that increase expression of the target gene in the assay.

In some embodiments, the oligonucleotide is not complementary to a sequence of FAST-1 antisense RNA. In some embodiments, the oligonucleotide is not complementary to the sequence in International Patent Application Publication WO12170771A1 that is identified as SEQ ID NO: 2.

### Oligonucleotides for Increasing Gene Expression

In one aspect, the invention relates to methods for increasing gene expression in a cell for research purposes (e.g., to study the function of the gene in the cell that is silenced or downregulated due to heterochromatin formation). In another aspect, the invention relates to methods for increasing gene expression in a cell for therapeutic purposes. The cells can be in vitro, ex vivo, or in vivo (e.g., in a subject in need thereof, such a as a subject who has a disease resulting from reduced expression or activity of a target gene). In some embodiments, methods for increasing gene expression in a cell comprise delivering an oligonucleotide as described herein. In some embodiments, gene expression is increased by at least 5%, 10%, 20%, 30%, 40%, 50%, 60%, 70%, 80%, 90%, 100%, 200% or more greater than gene expression in a control cell or control subject. An appropriate control cell or subject may be a cell, tissue or subject to which an oligonucleotide has not been delivered or to which a negative control has been delivered (e.g., a scrambled oligo, a carrier, etc.). In some embodiments, gene expression includes an increase of protein expression by at least 5%, 10%, 20%, 30%, 40%, 50%, 60%, 70%, 80%, 90%, 100%, 200%, or more, higher than the amount of a protein in the subject (e.g., in a cell or tissue of the subject) before administering an oligonucleotide or in a control subject which has not been administered the oligonucleotide or that has been administered a negative control (e.g., a scrambled oligo, a carrier, etc.).

In some embodiments, methods are provided for treating a disease associated with repeat expansion in a gene. Typically, the methods involve administering to a subject an effective amount of an oligonucleotide for increasing expression of the gene. In some embodiments, the oligonucleotide is a gapmer that is complementary to a repetitive sequence in a non-coding RNA or a complement thereof, the repetitive sequence being a repeating set of nucleotides wherein the set is 3-5 nucleotides in length and includes at least 2, at least 4, at least 6, at least 8, or at least 10 repeats.

In some embodiments, the disease associated with heterochromatin regulation (e.g., due to repetitive sequences) is selected from Angelman syndrome, myotonic dystrophy type 1, Friedreich's ataxia, fragile x syndrome, Prader-Willi syndrome and cancer associated with heterochromatin silencing of tumor suppressor genes.

It is understood that any reference to uses of compounds throughout the description contemplates use of the compound in preparation of a pharmaceutical composition or medicament for use in the treatment of condition or a disease. Thus, as one non-limiting example, this aspect of the invention includes use of such oligonucleotides in the preparation of a medicament for use in the treatment of disease associated with heterochromatin regulation.

It should be appreciated that oligonucleotides provided herein for increasing gene expression may be single stranded or double stranded. Single stranded oligonucleotides may include secondary structures, e.g., a loop or helix structure, and thus may have one or more double stranded portions under certain physiochemical conditions. In some embodiments, the oligonucleotide comprises at least one modified nucleotide or modified internucleoside linkage as described herein.

Oligonucleotides provided herein may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches may have increased non-specific binding and/or off-target effects, compared with oligonucleotides that do not have guanosine nucleotide stretches.

Oligonucleotides provided herein may have a sequence that has less than a threshold level of sequence identity with every sequence of nucleotides, of equivalent length, that map to a genomic position encompassing or in proximity to an off-target gene. For example, an oligonucleotide may be designed to ensure that it does not have a sequence that maps to genomic positions encompassing or in proximity with all known genes (e.g., all known protein coding genes) other than a target gene. The threshold level of sequence identity may be 50%, 60%, 70%, 80%, 85%, 90%, 95%, 99% or 100% sequence identity.

Oligonucleotides provided herein may have a sequence that is has greater than 30% G-C content, greater than 40% G-C content, greater than 50% G-C content, greater than 60% G-C content, greater than 70% G-C content, or greater than 80% G-C content. The oligonucleotide may have a sequence that has up to 100% G-C content, up to 95% G-C content, up to 90% G-C content, or up to 80% G-C content. In some embodiments in which the oligonucleotide is 8 to 10 nucleotides in length, all but 1, 2, 3, 4, or 5 of the nucleotides are cytosine or guanosine nucleotides. In some embodiments, the sequence of the mRNA to which the oligonucleotide is complementary comprises no more than 3 nucleotides selected from adenine and uracil.

Oligonucleotides provided herein may be complementary to a target gene of multiple different species (e.g., human, mouse, rat, rabbit, goat, monkey, etc.). Oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of an oligonucleotide is complementary with at least 5 to 15, 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a heterochromatin forming non-coding RNA or reverse complementary sequence thereof. In some embodiments, the region of complementarity is complementary with at least 5 or at least 8 consecutive nucleotides of a heterochromatin forming non-coding RNA or reverse complementary sequence thereof. In some embodiments, oligonucleotide comprises a region of complementarity that hybridizes with an RNA transcript or DNA strand, or a portion of either one, said portion having a length of about 5 to 40, or about 8 to 40, or about 5 to 15, or about 5 to 30, or about 5 to 40, or about 5 to 50 contiguous nucleotides.

Complementary, as the term is used in the art, refers to the capacity for precise pairing between two nucleotides. For example, if a nucleotide at a certain position of an oligonucleotide is capable of hydrogen bonding with a nucleotide at the same position of a target nucleic acid (e.g., an RNA transcript, DNA strand), then the oligonucleotide and the target nucleic acid are considered to be complementary to each other at that position. The oligonucleotide and the target nucleic acid are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide and its target nucleic acid. For example, if a base at one position of an oligonucleotide is capable of hydrogen bonding with a base at the corresponding position of a target nucleic acid, then the bases are considered to be complementary to each other at that position. 100% complementarity is not required.

The oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a target nucleic acid. In some embodiments the oligonucleotide may contain 1, 2 or 3 base mismatches compared to the portion of the consecutive nucleotides of a target nucleic acid. In some embodiments the oligonucleotide may have up to 3 mismatches over 15 bases, or up to 2 mismatches over 10 bases.

It is understood in the art that a complementary nucleotide sequence need not be 100% complementary to that of its target nucleic acid to be specifically hybridizable or specific for a target nucleic acid. In some embodiments, a complementary nucleic acid sequence for purposes of the present disclosure is specifically hybridizable or specific for the target nucleic when binding of the sequence to the target nucleic acid (e.g., RNA transcript, DNA strand) results in increased expression of a target gene and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, the oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides in length. In a preferred embodiment, the oligonucleotide is 8 to 30 nucleotides in length.

Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, GC content of the oligonucleotide is preferably between about 30-60%. Contiguous runs of three or more Gs or Cs may not be preferable in some embodiments. Accordingly, in some embodiments, the oligonucleotide does not comprise a stretch of three or more guanosine nucleotides.

It is to be understood that any oligonucleotide provided herein can be excluded.

In some embodiments, it has been found that oligonucleotides disclosed herein may increase expression of a target gene by at least about 50% (i.e. 150% of normal or 1.5 fold), or by about 2 fold to about 5 fold. In some embodiments, expression may be increased by at least about 15 fold, 20 fold, 30 fold, 40 fold, 50 fold or 100 fold, or any range between any of the foregoing numbers.

The oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not mediate alternative splicing; are not immune stimulatory; are nuclease resistant; have improved cell uptake compared to unmodified oligonucleotides; are not toxic to cells or mammals; or have improved endosomal exit.

Any of the oligonucleotides disclosed herein may be linked to one or more other oligonucleotides disclosed herein by a linker, e.g., a cleavable linker.

Oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleoside linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom.

Any of the modified chemistries or formats of oligonucleotides described herein can be combined with each other, and that one, two, three, four, five, or more different types of modifications can be included within the same molecule.

In some embodiments, an oligonucleotide may comprise one or more modified nucleotides (also referred to herein as nucleotide analogs). In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often the oligonucleotide has one or more nucleotide analogues. For example, the oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. The oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues.

The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

Preferably the oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligonucleotides of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligonucleotide comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO/2008/043753 and include compounds of the following general formula.

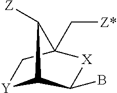

where X and Y are independently selected among the groups —O—,

—S—, —N(H)—, N(R)—, —CH₂— or —CH— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

In some embodiments, the LNA used in the oligonucleotides described herein comprises at least one LNA unit according any of the formulas

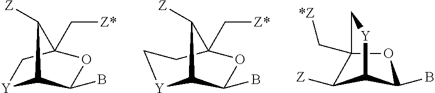

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the invention comprises internucleoside linkages selected from -0-P(O)₂—O—, —O—P(O,S)—O—, —0-P(S)₂—O—, —S—P(O)₂—O—, —S—P(O,S)—O—, —S—P(S)₂—O—, —0-P(O)₂—S—, —O—P(O,S)—S—, —S—P(O)₂—S—, —O—PO(R^{H})—O—, 0-PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown below:

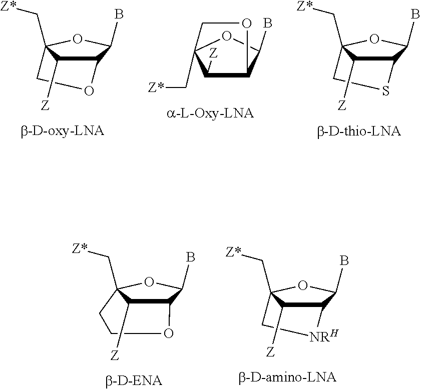

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃OCH₃, OCH₃O(CH₂)nCH₃, O(CH₂)nNH₂ or O(CH₂)nCH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH2; heterocycloalkyl; heterocycloalkaryl; amino alkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy[2′-O—CH₂CH₂OCH₃, also known as 2′-O-(2-methoxyethyl)] (Martin et al, Helv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S— tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, oligonucleotide modification includes modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of the oligonucleotide. In some embodiments, the oligonucleotide comprises a biotin moiety conjugated to the 5′ nucleotide.

In some embodiments, the oligonucleotide comprises locked nucleic acids (LNA), ENA modified nucleotides, 2′-O-methyl nucleotides, or 2′-fluoro-deoxyribonucleotides. In some embodiments, the oligonucleotide comprises alternating deoxyribonucleotides and 2′-fluoro-deoxyribonucleotides. In some embodiments, the oligonucleotide comprises alternating deoxyribonucleotides and 2′-O-methyl nucleotides. In some embodiments, the oligonucleotide comprises alternating deoxyribonucleotides and ENA modified nucleotides. In some embodiments, the oligonucleotide comprises alternating deoxyribonucleotides and locked nucleic acid nucleotides. In some embodiments, the oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides.

It should be appreciated that the oligonucleotide can have any combination of modifications as described herein.

In some embodiments, an oligonucleotide described herein may be a mixmer or comprise a mixmer sequence pattern. The term ‘mixmer’ refers to oligonucleotides which comprise both naturally and non-naturally occurring nucleotides or comprise two different types of non-naturally occurring nucleotides. Mixmers are generally known in the art to have a higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule. Accordingly, in some embodiments, an oligonucleotide provided herein may be cleavage promoting (e.g., an siRNA or gapmer) or not cleavage promoting (e.g., a mixmer, siRNA, single stranded RNA or double stranded RNA).

In some embodiments, the mixmer comprises or consists of a repeating pattern of nucleotide analogues and naturally occurring nucleotides, or one type of nucleotide analogue and a second type of nucleotide analogue. However, it is to be understood that the mixmer need not comprise a repeating pattern and may instead comprise any arrangement of nucleotide analogues and naturally occurring nucleotides or any arrangement of one type of nucleotide analogue and a second type of nucleotide analogue. The repeating pattern, may, for instance be every second or every third nucleotide is a nucleotide analogue, such as LNA, and the remaining nucleotides are naturally occurring nucleotides, such as DNA, or are a 2′ substituted nucleotide analogue such as 2′MOE or 2′ fluoro analogues, or any other nucleotide analogues described herein. It is recognised that the repeating pattern of nucleotide analogues, such as LNA units, may be combined with nucleotide analogues at fixed positions—e.g. at the 5′ or 3′ termini.

In some embodiments, the mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive nucleotide analogues, such as at least two consecutive LNAs. In some embodiments, the mixmer comprises at least a region consisting of at least three consecutive nucleotide analogue units, such as at least three consecutive LNAs.

In some embodiments, the mixmer does not comprise a region of more than 7, more than 6, more than 5, more than 4, more than 3, or more than 2 consecutive nucleotide analogues, such as LNAs. It is to be understood that the LNA units may be replaced with other nucleotide analogues, such as those referred to herein.

In some embodiments, the mixmer comprises at least one nucleotide analogue in one or more of six consecutive nucleotides. The substitution pattern for the nucleotides may be selected from the group consisting of Xxxxxx, xXxxxx, xxXxxx, xxxXxx, xxxxXx and xxxxxX, wherein “X” denotes a nucleotide analogue, such as an LNA, and “x” denotes a naturally occurring nucleotide, such as DNA or RNA.

In some embodiments, the mixmer comprises at least two nucleotide analogues in one or more of six consecutive nucleotides. The substitution pattern for the nucleotides may be selected from the group consisting of XXxxxx, XxXxxx, XxxXxx, XxxxXx, XxxxxX, xXXxxx, xXxXxx, xXxxXx, xXxxxX, xxXXxx, xxXxXx, xxXxxX, xxxXXx, xxxXxX and xxxxXX, wherein “X” denotes a nucleotide analogue, such as an LNA, and “x” denotes a naturally occurring nucleotide, such as DNA or RNA. In some embodiments, the substitution pattern for the nucleotides may be selected from the group consisting of XxXxxx, XxxXxx, XxxxXx, XxxxxX, xXxXxx, xXxxXx, xXxxxX, xxXxXx, xxXxxX and xxxXxX. In some embodiments, the substitution pattern is selected from the group consisting of xXxXxx, xXxxXx, xXxxxX, xxXxXx, xxXxxX and xxxXxX. In some embodiments, the substitution pattern is selected from the group consisting of xXxXxx, xXxxXx and xxXxXx. In some embodiments, the substitution pattern for the nucleotides is xXxXxx.

In some embodiments, the mixmer comprises at least three nucleotide analogues in one or more of six consecutive nucleotides. The substitution pattern for the nucleotides may be selected from the group consisting of XXXxxx, xXXXxx, xxXXXx, xxxXXX, XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx, wherein “X” denotes a nucleotide analogue, such as an LNA, and “x” denotes a naturally occurring nucleotide, such as DNA or RNA. In some embodiments, the substitution pattern for the nucleotides is selected from the group consisting of XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx. In some embodiments, the substitution pattern for the nucleotides is selected from the group consisting of xXXxXx, xXXxxX, xxXXxX, xXxXXx, xXxxXX, xxXxXX and xXxXxX. n some embodiments, the substitution pattern for the nucleotides is xXxXxX or XxXxXx. In some embodiments, the substitution pattern for the nucleotides is xXxXxX.

In some embodiments, the mixmer comprises at least four nucleotide analogues in one or more of six consecutive nucleotides. The substitution pattern for the nucleotides may be selected from the group consisting of xXXXX, xXxXXX, xXXxXX, xXXXxX, xXXXXx, XxxXXX, XxXxXX, XxXXxX, XxXXXx, XXxxXX, XXxXxX, XXxXXx, XXXxxX, XXXxXx and XXXXxx, wherein “X” denotes a nucleotide analogue, such as an LNA, and “x” denotes a naturally occurring nucleotide, such as DNA or RNA.

In some embodiments, the mixmer comprises at least five nucleotide analogues in one or more of six consecutive nucleotides. The substitution pattern for the nucleotides may be selected from the group consisting of xXXXXX, XxXXXX, XXxXXX, XXXxXX, XXXXxX and XXXXXx, wherein “X” denotes a nucleotide analogue, such as an LNA, and “x” denotes a naturally occurring nucleotide, such as DNA or RNA.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXXXX, (X)XXXXXX, (X)XXXXXX, (X)XXXXXX, (X)XXXXXX, (X)XXXXXX, (X)XXXXXX, (X)XXXXXX, (X)XXXXXX, (X)XXXXXX, (X)XXXXXX, (X)XXXXXX (X)XXXXXX, (X)XXXXXX, (X)XXXXXX, (X)XXXXXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXXXXX, (X)XXXXXX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

In some embodiments, the mixmer contains a modified nucleotide, e.g., an LNA, at the 5′ end. In some embodiments, the mixmer contains a modified nucleotide, e.g., an LNA, at the first two positions, counting from the 5′ end.

In some embodiments, the mixmer is incapable of recruiting RNAseH. Oligonucleotides that are incapable of recruiting RNAseH are well known in the literature, in example see WO2007/112754, WO2007/112753, or PCT/DK2008/000344. Mixmers may be designed to comprise a mixture of affinity enhancing nucleotide analogues, such as in non-limiting example LNA nucleotides and 2′-O-methyl nucleotides. In some embodiments, the mixmer comprises modified internucleoside linkages (e.g., phosphorothioate internucleoside linkages or other linkages) between at least two, at least three, at least four, at least five or more nucleotides.

A mixmer may be produced using any method known in the art or described herein. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, the oligonucleotide is a gapmer. A gapmer oligonucleotide generally has the formula 5′-X—Y—Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAseH. Without wishing to be bound by theory, it is thought that the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., 1-6 modified nucleotides. Exemplary modified oligonucleotides include, but are not limited to, 2′ MOE or 2′OMe or Locked Nucleic Acid bases (LNA). The flanks X and Z may be have a of length 1-20 nucleotides, preferably 1-8 nucleotides and even more preferred 1-5 nucleotides. The flanks X and Z may be of similar length or of dissimilar lengths. The gap-segment Y may be a nucleotide sequence of length 5-20 nucleotides, preferably 6-12 nucleotides and even more preferred 6-10 nucleotides. In some aspects, the gap region of the gapmer oligonucleotides of the invention may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using any method known in the art or described herein. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

In some embodiments, oligonucleotides provided herein may be in the form of small interfering RNAs (siRNA), also known as short interfering RNA or silencing RNA. SiRNA, is a class of RNA molecules (e.g., double stranded), typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective.

Following selection of an appropriate target RNA sequence, siRNA molecules that comprise a nucleotide sequence complementary to all or a portion of the target sequence, i.e. an antisense sequence, can be designed and prepared using any method known in the art (see, e.g., PCT Publication Nos. WO08124927A1 and WO 2004/016735; and U.S. Patent Publication Nos. 2004/0077574 and 2008/0081791). A number of commercial packages and services are available that are suitable for use for the preparation of siRNA molecules. These include the in vitro transcription kits available from Ambion (Austin, Tex.) and New England Biolabs (Beverly, Mass.) as described above; viral siRNA construction kits commercially available from Invitrogen (Carlsbad, Calif.) and Ambion (Austin, Tex.), and custom siRNA construction services provided by Ambion (Austin, Tex.), Qiagen (Valencia, Calif.), Dharmacon (Lafayette, Colo.) and Sequitur, Inc (Natick, Mass.). A target sequence can be selected (and a siRNA sequence designed) using computer software available commercially (e.g. OligoEngine™ (Seattle, Wash.); Dharmacon, Inc. (Lafayette, Colo.); Target Finder from Ambion Inc. (Austin, Tex.) and the siRNA Design Tool from QIAGEN, Inc. (Valencia, Calif.)). In some embodiments, an siRNA may be designed or obtained using the RNAi atlas (available at the RNAiAtlas website), the siRNA database (available at the Stockholm Bioinformatics Website), or using DesiRM (available at the Institute of Microbial Technology web site).

The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). A spacer sequence is may be an unrelated nucleotide sequence that is situated between two complementary nucleotide sequence regions which, when annealed into a double-stranded nucleic acid, comprise a shRNA.

The overall length of the siRNA molecules can vary from about 14 to about 200 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 200 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present invention comprises 3′ overhangs of about 1 to about 3 nucleotides on both ends of the molecule.

In some embodiments, an oligonucleotide may be a microRNA (miRNA). MicroRNAs (referred to as “miRNAs”) are small non-coding RNAs, belonging to a class of regulatory molecules found in plants and animals that control gene expression by binding to complementary sites on a target RNA transcript. miRNAs are generated from large RNA precursors (termed pri-miRNAs) that are processed in the nucleus into approximately 70 nucleotide pre-miRNAs, which fold into imperfect stem-loop structures (Lee, Y., et al., Nature (2003) 425(6956):415-9). The pre-miRNAs undergo an additional processing step within the cytoplasm where mature miRNAs of 18-25 nucleotides in length are excised from one side of the pre-miRNA hairpin by an RNase III enzyme, Dicer (Hutvagner, G., et al., Science (2001) 12:12 and Grishok, A., et al., Cell (2001) 106(1):23-34).

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides, although miRNAs of up to 2000 nucleotides can be utilized. In a preferred embodiment the size range of the miRNA is from 70 to 170 nucleotides in length. In another preferred embodiment, mature miRNAs of from 21 to 25 nucleotides in length can be used.

In some embodiments, the miRNA may be a miR-30 precursor. As used herein, an “miR-30 precursor”, also called an miR-30 hairpin, is a precursor of the human microRNA miR-30, as it is understood in the literature (e.g., Zeng and Cullen, 2003; Zeng and Cullen, 2005; Zeng et al., 2005; United States Patent Application Publication No. US 2004/005341), where the precursor could be modified from the wild-type miR-30 precursor in any manner described or implied by that literature, while retaining the ability to be processed into an miRNA. In some embodiments, a miR-30 precursor is at least 80 nucleotides long and comprises a stem-loop structure. In some embodiments, the miR-30 precursor further comprises a first miRNA sequence of 20-22 nucleotides on the stem of the stem-loop structure complementary to a portion of a first target sequence.

A miRNA may be isolated from a variety of sources or may be synthesized according to methods well known in the art (see, e.g., Current Protocols in Molecular Biology, Wiley Online Library; U.S. Pat. No. 8,354,384; and Wahid et al. MicroRNAs: synthesis, mechanism, function, and recent clinical trials. Biochim Biophys Acta. 2010; 1803(11):1231-43). In some embodiments, a miRNA is expressed from a vector as known in the art or described herein. In some embodiments, the vector may include a sequence encoding a mature miRNA. In some embodiments, the vector may include a sequence encoding a pre-miRNA such that the pre-miRNA is expressed and processed in a cell into a mature miRNA. In some embodiments, the vector may include a sequence encoding a pri-miRNA. In this embodiment, the primary transcript is first processed to produce the stem-loop precursor miRNA molecule. The stem-loop precursor is then processed to produce the mature microRNA.

In some embodiments, oligonucleotides provided herein may be in the form of aptamers. An “aptamer” is any nucleic acid that binds specifically to a target, such as a small molecule, protein, nucleic acid, cell, tissue or organism. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof.

Selection of nucleic acid aptamers may be accomplished by any suitable method known in the art, including an optimized protocol for in vitro selection, known as SELEX (Systemic Evolution of Ligands by Exponential enrichment). Many factors are important for successful aptamer selection. For example, the target molecule should be stable and easily reproduced for each round of SELEX, because the SELEX process involves multiple rounds of binding, selection, and amplification to enrich the nucleic acid molecules. In addition, the nucleic acids that exhibit specific binding to the target molecule have to be present in the initial library. Thus, it is advantageous to produce a highly diverse nucleic acid pool. Because the starting library is not guaranteed to contain aptamers to the target molecule, the SELEX process for a single target may need to be repeated with different starting libraries. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′,5′-phosphate diester to a 2′,3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

In some embodiments, the oligonucleotide does not comprise a pseudoisocytosine. In some embodiments, the oligonucleotide does not comprise a PNA. In some embodiments, the oligonucleotide does not comprise a LNA. In some embodiments, the oligonucleotide does not consists of all PNAs or all LNAs. In some embodiments, the oligonucleotide is not a morpholino.

### Formulation, Delivery, And Dosing

The oligonucleotides described herein can be formulated for administration to a subject for treating a condition associated with decreased levels of a target gene due to heterochromatin formation (e.g., resulting from non-coding RNAs containing repetitive sequences). It should be understood that the formulations, compositions and methods can be practiced with any of the oligonucleotides disclosed herein.

The formulations may conveniently be presented in unit dosage form and may be prepared by any methods well known in the art of pharmacy. The amount of active ingredient (e.g., an oligonucleotide or compound of the invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g. tumor regression.

Pharmaceutical formulations of this invention can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated oligonucleotide composition can assume a variety of states. In some examples, the composition is at least partially crystalline, uniformly crystalline, and/or anhydrous (e.g., less than 80, 50, 30, 20, or 10% water). In another example, the oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide preparation can be formulated or administered (together or separately) in combination with another agent, e.g., another therapeutic agent or an agent that stabilizes an oligonucleotide, e.g., a protein that complexes with the oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the oligonucleotide preparation includes another oligonucleotide, e.g., a second oligonucleotide that modulates expression of a second gene or a second oligonucleotide that modulates expression of the first gene. Still other preparation can include at least 3, 5, ten, twenty, fifty, or a hundred or more different oligonucleotide species. Such oligonucleotides can mediated gene expression with respect to a similar number of different genes. In one embodiment, the oligonucleotide preparation includes at least a second therapeutic agent (e.g., an agent other than an oligonucleotide).

### Route of Delivery

A composition that includes an oligonucleotide can be delivered to a subject by a variety of routes. Exemplary routes include: intrathecal, intraneural, intracerebral, intramuscular, oral, intravenous, intradermal, topical, rectal, parenteral, anal, intravaginal, intranasal, pulmonary, or ocular. The term “therapeutically effective amount” is the amount of oligonucleotide present in the composition that is needed to provide the desired level of gene expression in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

The oligonucleotide molecules of the invention can be incorporated into pharmaceutical compositions suitable for administration. Such compositions typically include one or more species of oligonucleotide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

In some embodiments, the oligonucleotide is prepared in a pharmaceutical composition at a concentration of less than 5 mg/ml. In some embodiments, the oligonucleotide is prepared in a pharmaceutical composition at a concentration of greater than 50 mg/ml. In some embodiments, the oligonucleotide is prepared in a pharmaceutical composition at a concentration in a range of greater than 50 mg/ml to 500 mg/ml or more.

The route and site of administration may be chosen to enhance targeting. For example, to target muscle cells, intramuscular injection into the muscles of interest would be a logical choice. Lung cells might be targeted by administering the oligonucleotide in aerosol form. The vascular endothelial cells could be targeted by coating a balloon catheter with the oligonucleotide and mechanically introducing the oligonucleotide. Targeting of neuronal cells could be accomplished by intrathecal, intraneural, intracerebral administration.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, oligonucleotides administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the oligonucleotide can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A pharmaceutical composition of oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the oligonucleotides described herein can be administered to ocular tissue. For example, the compositions can be applied to the surface of the eye or nearby tissue, e.g., the inside of the eyelid. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The oligonucleotide can also be administered to the interior of the eye, and can be introduced by a needle or other delivery device which can introduce it to a selected area or structure.

Pulmonary delivery compositions can be delivered by inhalation by the patient of a dispersion so that the composition, preferably oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. A oligonucleotide composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an oligonucleotide, e.g., a device can release insulin.

In one embodiment, unit doses or measured doses of a composition that includes oligonucleotide are dispensed by an implanted device. The device can include a sensor that monitors a parameter within a subject. For example, the device can include pump, e.g., and, optionally, associated electronics.

Tissue, e.g., cells or organs can be treated with an oligonucleotide, ex vivo and then administered or implanted in a subject. The tissue can be autologous, allogeneic, or xenogeneic tissue. E.g., tissue can be treated to reduce graft v. host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, the oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

### Dosage

In one aspect, the invention features a method of administering an oligonucleotide (e.g., as a compound or as a component of a composition) to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or disorder, e.g., a disease or disorder associated with a reduced level of a target gene. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of an oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. In some embodiments, the oligonucleotide is administered to a subject at a concentration of less than 0.1 mg/kg. In some embodiments, the oligonucleotide is administered to a subject at a concentration of greater than 0.6 mg/kg. In some embodiments, the oligonucleotide is administered to a subject at a concentration of greater than 0.6 mg/kg to 100 mg/kg.

Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, oligonucleotide pharmaceutical compositions are provided that include a plurality of oligonucleotides. In some embodiments, oligonucleotides in the plurality have sequences that are non-overlapping and non-adjacent to other oligonucleotides in the plurality with respect to a target gene sequence. In some embodiments, the plurality contains oligonucleotides specific for different target genes. In some embodiments, the plurality contains oligonucleotides that are allele specific.

In some cases, a patient is treated with an oligonucleotide in conjunction with other therapeutic modalities.

Following successful treatment, it may be desirable to have the patient undergo maintenance therapy to prevent the recurrence of the disease state, wherein the compound of the invention is administered in maintenance doses, ranging from 0.0001 mg to 100 mg per kg of body weight.

The concentration of the oligonucleotide composition is an amount sufficient to be effective in treating or preventing a disorder or to regulate a physiological condition in humans. The concentration or amount of oligonucleotide administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an oligonucleotide can include a single treatment or, preferably, can include a series of treatments. It will also be appreciated that the effective dosage of an oligonucleotide used for treatment may increase or decrease over the course of a particular treatment. For example, the subject can be monitored after administering an oligonucleotide composition. Based on information from the monitoring, an additional amount of the oligonucleotide composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of gene expression levels in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that are engineered to express a human gene. In another embodiment, the composition for testing includes an oligonucleotide that is complementary, at least in an internal region, to a sequence that is conserved between gene in the animal model and the corresponding gene in a human.

In one embodiment, the administration of the oligonucleotide composition is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The composition can be provided in measured doses or in a dispenser which delivers a metered dose. Selected modes of delivery are discussed in more detail below.

### Kits

In certain aspects of the invention, kits are provided, comprising a container housing a composition comprising an oligonucleotide. In some embodiments, the composition is a pharmaceutical composition comprising an oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present invention is further illustrated by the following Examples, which in no way should be construed as further limiting.

### EXAMPLES

### Example 1

### Materials and Methods

### Real Time PCR

RNA analysis, cDNA synthesis and QRT-PCR was done with Life Technologies Cells-to-Ct kit and StepOne Plus instrument. Baseline levels were also determined for mRNA of various housekeeping genes which are constitutively expressed. A “control” housekeeping gene with approximately the same level of baseline expression as the target gene was chosen for comparison purposes. FXN and control (ACTIN) Taqman primers were purchased from Life Technologies.

### Cell Lines

Cells were cultured using conditions known in the art (see, e.g. Current Protocols in Cell Biology). Details of the cell lines used in the experiments described herein are provided in Table 2.

TABLE 2
Cell lines

| | Clinically | | # of GAA | |
|---|---|---|---|---|
| Cell lines | affected | Cell type | repeats | Notes |
| GM15850 | Y | B-lymphoblast | 650 & 1030 | 13 yr old white |
| | | | | male, brother |
| | | | | to GM15851 |
| GM15851 | N | B-lymphoblast | <20 for both | 14 yr old white |
| | | | | male, brother |
| | | | | to GM15850 |
| GM16209 | Y | B-lymphoblast | 800 for both | 41 yr old white |
| | | | | female, half-sister |
| | | | | to GM16222 |
| GM16228 | Y | B-lymphoblast | 830 and 670 | 21 yr old white |
| | | | | female |
| GM03816 | Y | Fibroblast | 330 and 380 | 36 yr old white |
| | | | | female |

### Identification of RNA Transcripts in the First FXN Intron

RNA sequencing was performed on RNA extracted from each of the cell lines GM15850, GM15851, GM16209, and GM16228. The sequencing was done using the Illumina Hi-Seq system with 100 nt paired reads. The quality filtered data was aligned with Tophat using the human hg19 reference genome with and without supplemented GAA-repeat track in the mutation location in the FXN first intron. The differences in alignment between the references with and without GAA-repeats were quantified.

### Oligonucleotide Design

Gapmer oligonucleotides were designed to target the GAA repeat region present in the first intron of the FXN gene. Specifically, gapmer oligonucleotides were designed to target the sense GAA repeat sequence and the anti-sense TTC repeat sequence. The sequence and structure of each gapmer oligonucleotide is shown in Table 3. Table 4 provides a description of the nucleotide analogs, modifications and intranucleotide linkages used for certain oligonucleotides tested and described in Table 3.

TABLE 3
Oligonucleotides designed to target
the GAA repeat region

| SEQ | | | | |
|---|---|---|---|---|
| ID NO | Base sequence | Gene | Species | Formatted sequence |
| 1 | GAAGAAGA | FXN | Human | lnaGs; lnaAs; |
| | AGAAGAA | | | lnaAs; dGs; |
| | | | | dAs; dAs; |
| | | | | dGs; dAs; |
| | | | | dAs; dGs; |
| | | | | dAs; dAs; |
| | | | | lnaGs; lnaAs; |
| | | | | lnaA-Sup |
| 2 | TTCTTCTTCT | FXN | Human | lnaTs; lnaTs; |
| | TCTTC | | | lnaCs; dTs; |
| | | | | dTs; dCs; |
| | | | | dTs; dTs; |
| | | | | dCs; dTs; |
| | | | | dTs; dCs; |
| | | | | lnaTs; lnaTs; |
| | | | | lnaC-Sup |

TABLE 4
Oligonucleotide Modifications

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| lnaAs | LNA w/3′ thiophosphate |
| lnaCs | LNA w/3′ thiophosphate |
| lnaGs | LNA w/3′ thiophosphate |
| lnaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| lnaAs-Sup | LNA w/3′ thiophosphate at 3′ terminus |
| lnaCs-Sup | LNA w/3′ thiophosphate at 3′ terminus |
| lnaGs-Sup | LNA w/3′ thiophosphate at 3′ terminus |
| lnaTs-Sup | LNA w/3′ thiophosphate at 3′ terminus |
| lnaA-Sup | LNA w/3′ OH at 3′ terminus |
| lnaC-Sup | LNA w/3′ OH at 3′ terminus |
| lnaG-Sup | LNA w/3′ OH at 3′ terminus |
| lnaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |

In Vitro Transfection of Cells with Oligonucleotides

Cells were seeded into each well of 96- and 6-well plates at a density of 5000 cells per 500 uL and 100000 cells per 2 ml, respectively, and transfections were performed with Lipofectamine 2000 and the single stranded oligonucleotides. Control wells contained Lipofectamine alone. RNA isolation and analyses were done with the Cells-to-Ct kit (Life Technologies) for the 96-wells, and Trizol (Sigma) for the 6-well experiments. The percent induction of target mRNA expression by each oligonucleotide was determined by normalizing mRNA levels in the presence of the oligonucleotide to the mRNA levels in the presence of control (Lipofectamine alone). ELISA for FXN was done using 6-well cell lysates following manufacturer's (Abcam) instructions.

### Results:

The frataxin (FXN) gene was selected as a candidate to determine if heterochromatin formation could be targeted using oligonucleotides in order to cause upregulation of FXN expression. Friedreich's Ataxia (FRDA) is an autosomal recessive disease characterized by onset of a progressive degenerative neuromuscular disorder. Frataxin, the gene implicated in FRDA, is highly expressed in heart, brain, spinal cord and voluntary skeletal muscle. FRDA patients have a GAA repeat expansion in FXN intron. It is believed that this GAA repeat expansion results in reduced transcription of FXN due to heterochromatic silencing and that this silencing is involved in the pathology of FRDA. As the FXN exons are normal in patients with FRDA, increased expression of the endogenous gene are expected to curative.

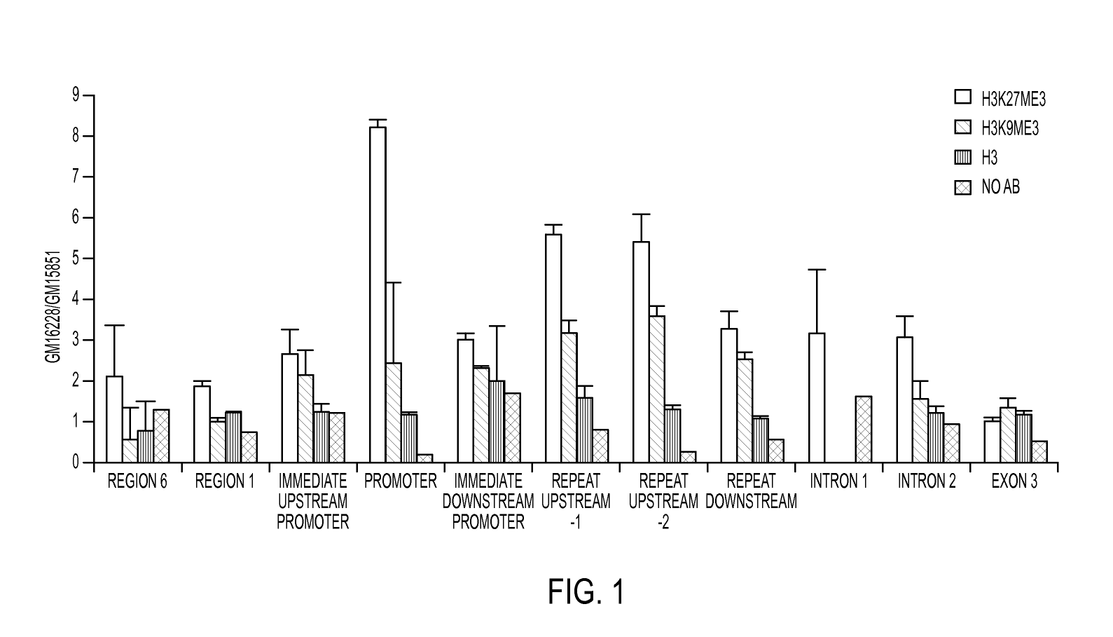

Cells from FRDA patients express heterochromatin markers characteristic of gene silencing. In the present study, the heterochromatin formation throughout the FXN gene locus was examined. It was found that heterochromatin-like structures occurred around the GAA repeat region in FRDA patient cells (FIG. 1).

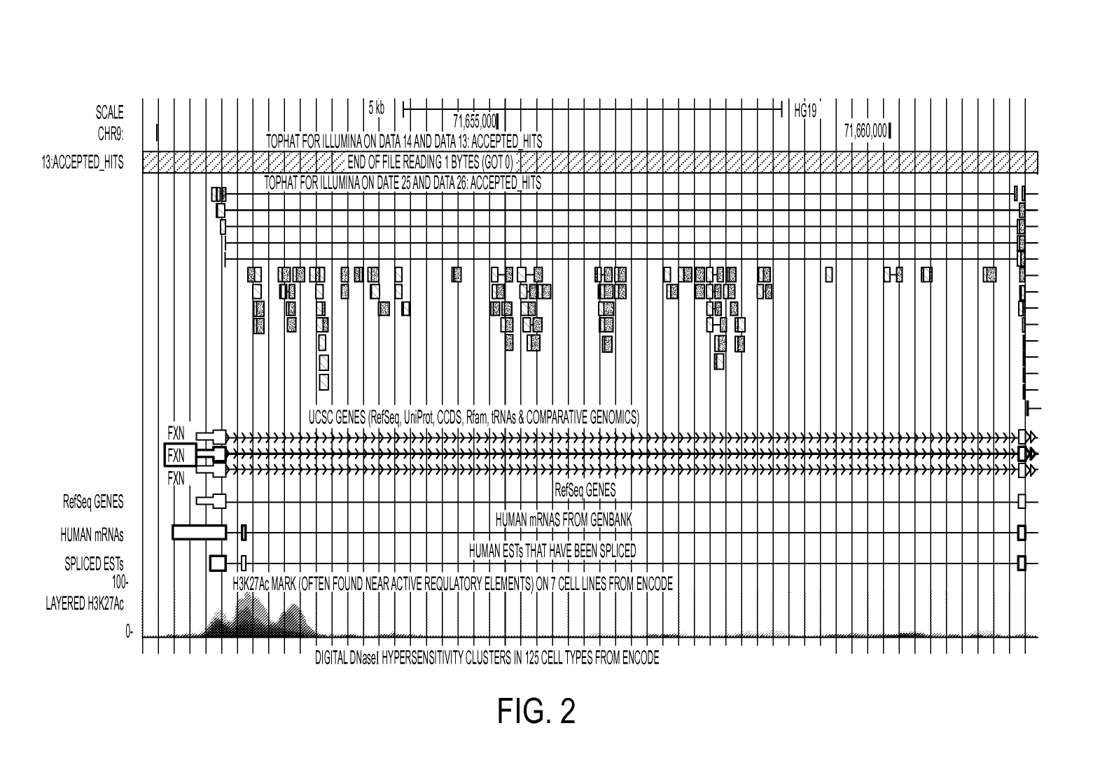

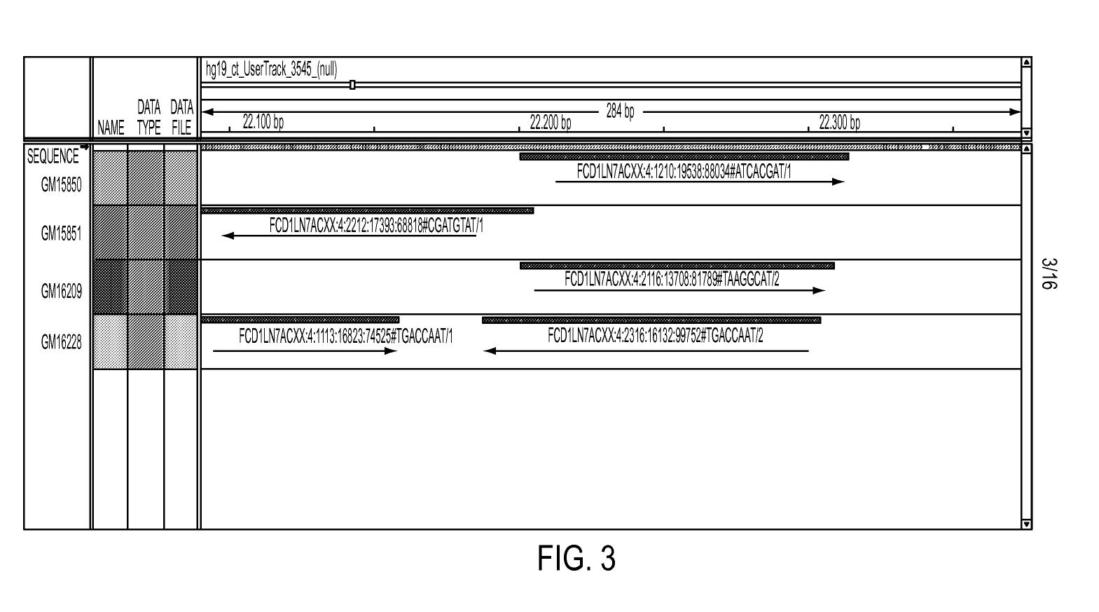

It was hypothesized that the observed heterochromatin formation at the FXN locus was RNAi-mediated heterochromatin formation. RNAi-mediated heterochromatin formation was believed to involve recruitment of an Argonaute-containing RITS complex, which then recruits a histone methyltransferase. Double-stranded RNAs are thought to be processed by Dicer to produce siRNAs. These siRNAs then bind to an RNA transcript and recruit the RITS complex. This recruitment results in H3 K9 methylation of the genomic DNA. To determine if such a mechanism could cause heterochromatin formation and subsequent inhibition of FXN expression at the FXN locus, the FXN gene was examined for the presence of RNA transcripts transcribed at or near the first intron. It was predicted that an RNA transcript was transcribed in the first intron of FXN based on RNA sequencing data generated from normal cells and cells from FRDA patients (FIGS. 2 and 3).

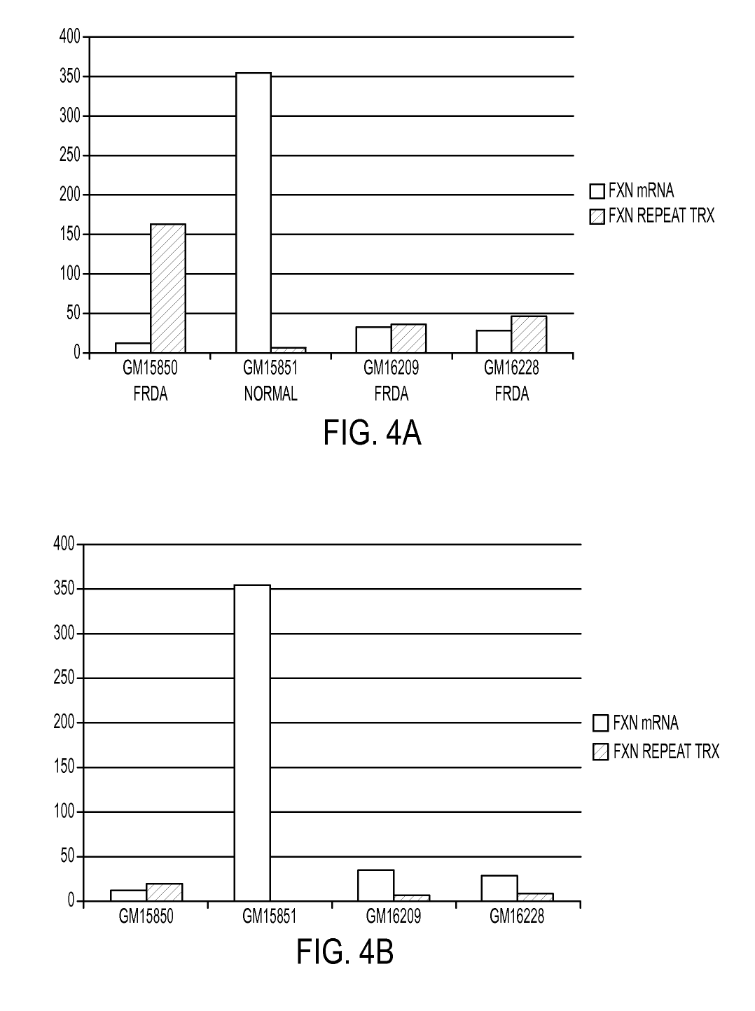

To further verify if RNA transcripts were transcribed at or near the first intron of FXN, qRT-PCR was performed to determine if an RNA containing the GAA repeat sequence was transcribed within the FXN gene. It was determined that an RNA transcript containing the GAA repeat was upregulated in cells from FRDA patients, but not in control cells (FIG. 4). Additionally, the GAA repeat RNA transcription levels and the FXN mRNA levels appeared to be inversely related. The inverse correlation suggested that GAA repeat RNA transcription may inhibit FXN mRNA transcription.

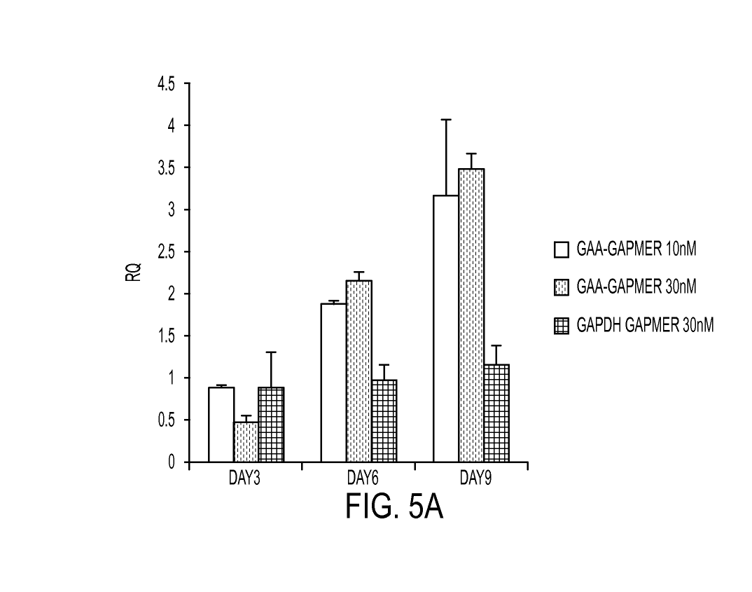

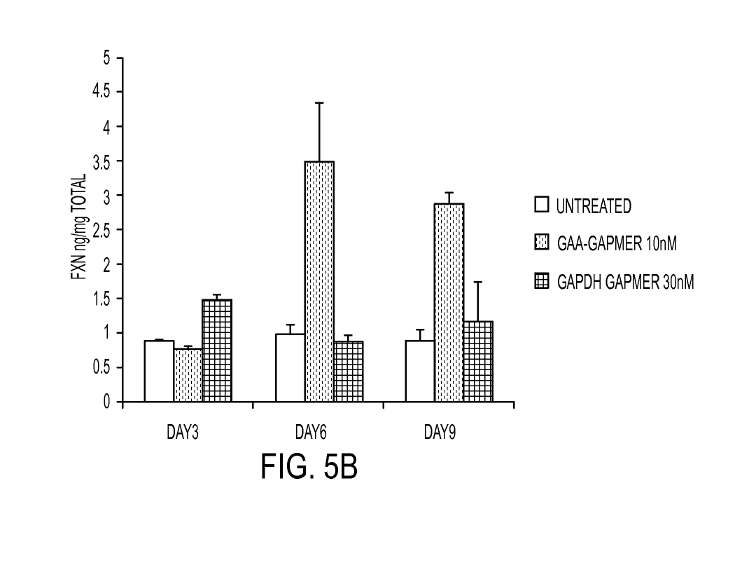

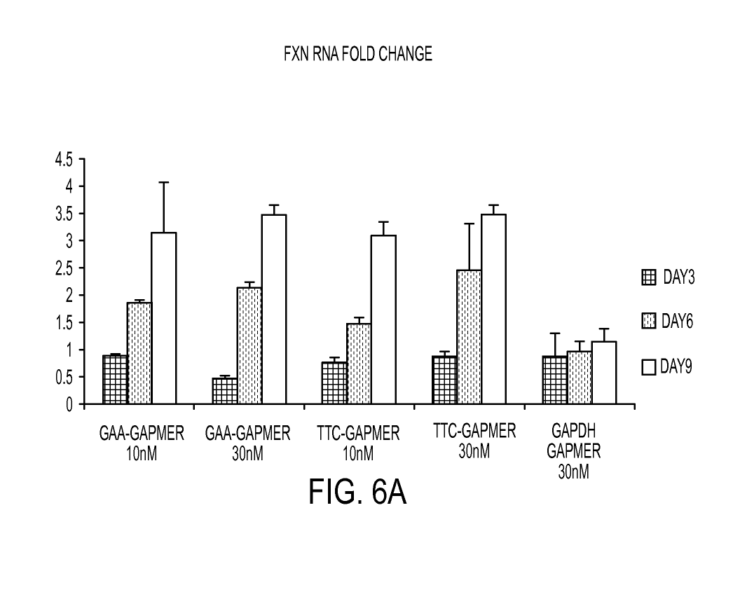

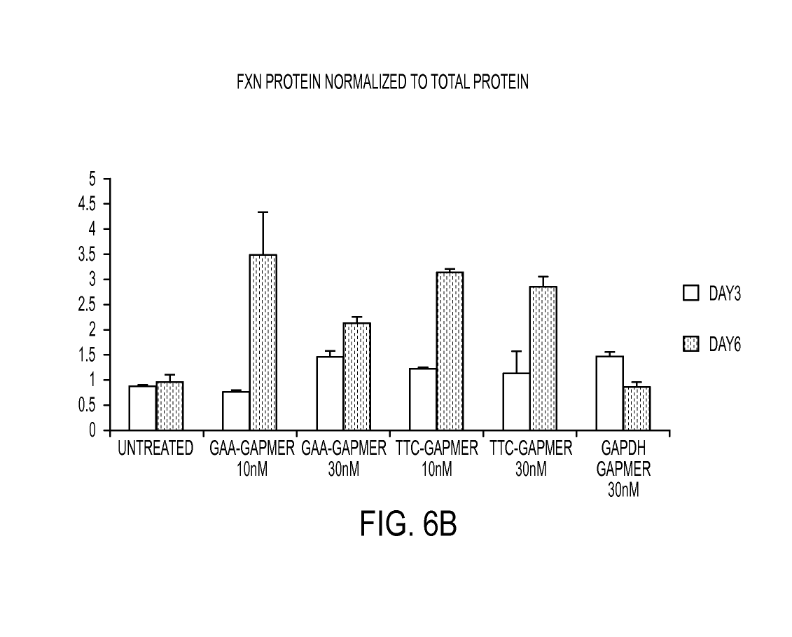

To determine if GAA repeat transcription caused inhibition of FXN mRNA, gapmers were designed to target the GAA repeat sequence and the anti-sense TTC repeat sequence. It was hypothesized that the gapmers would degrade the GAA repeat RNA transcript and/or cause steric hindrance by blocking the binding of the GAA repeat RNA to a complementary FXN intronic sequence. It was demonstrated that gapmers specific for the GAA repeat and the TTC repeat increased FXN mRNA levels and FXN protein levels (FIGS. 5 and 6). This data indicates that the GAA repeat RNA transcript present in the first intron inhibits FXN mRNA transcription, as treatment of FRDA cells with gapmers to the GAA repeat or the TTC repeat relieved the inhibition of FXN mRNA transcription. This data also supports the hypothesis that heterochromatin-mediated repression of a gene can be reversed by targeting an RNA transcript that may be involved in RNAi-mediated heterochromatin formation.

### Example 2

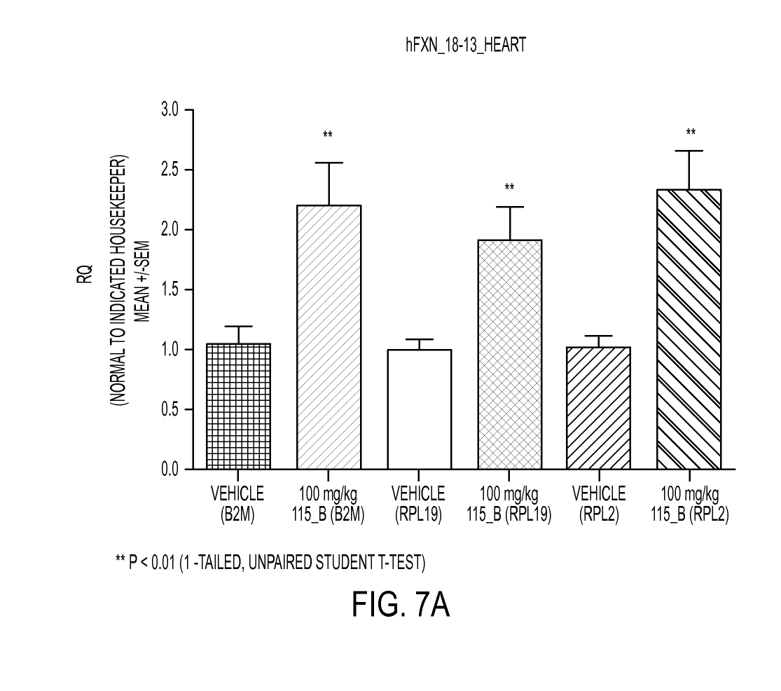

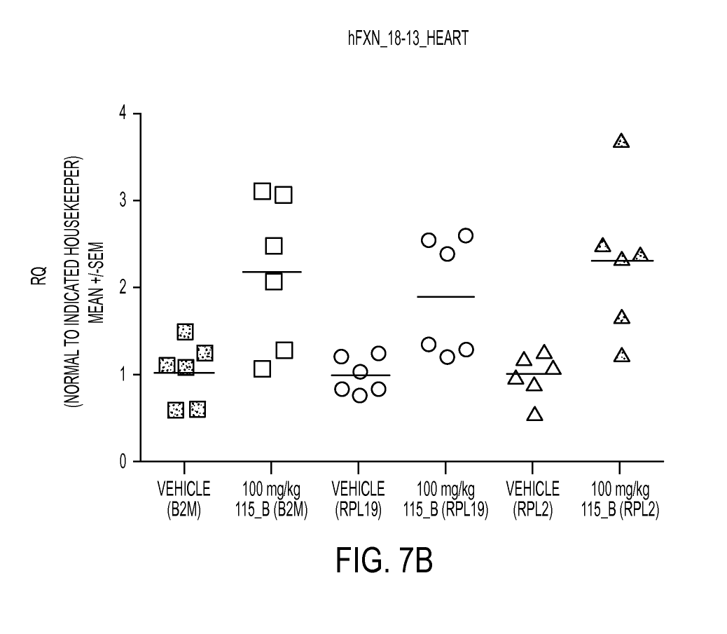

A GAA-repeat gapmer in Table 5 (FXN-115 m08, SEQ ID NO: 56, referred to as 115_B in FIGS. 7A and 7B) was used in the Sarsero mouse model of Friedreich's ataxia to measure upregulation of FXN in vivo.

The GAA-repeat gapmer was dissolved in PBS. The treatment group was injected subcutaneously with 100 mg/kg of the gapmer. The control group (vehicle) was injected with PBS. Both the treatment and vehicle groups had 6 mice each. The animals were 10-12 weeks old at the beginning of the study. The treatment period was 8-weeks, with administration of gapmer or vehicle on days 1, 2, 3 and then every 2nd week on days 15, 29, 43 & 57). Hearts from animals were collected 24 hours after the last dose. Human FXN RNA levels were measured using real-time PCR as described in Example 1 and normalized to three housekeepers (B2M, RPL19 & RPL2). FIG. 7A shows that the treatment group had elevated levels of FXN in the heart compared to the level of FXN in the vehicle group. FIG. 7B shows the level of FXN in each animal from the treatment or vehicle group. Most of the animals in the treatment group had an elevated level of FXN compared to the vehicle group. These data show that the effects demonstrated in Example 1 could also be achieved in vivo.

### Example 3

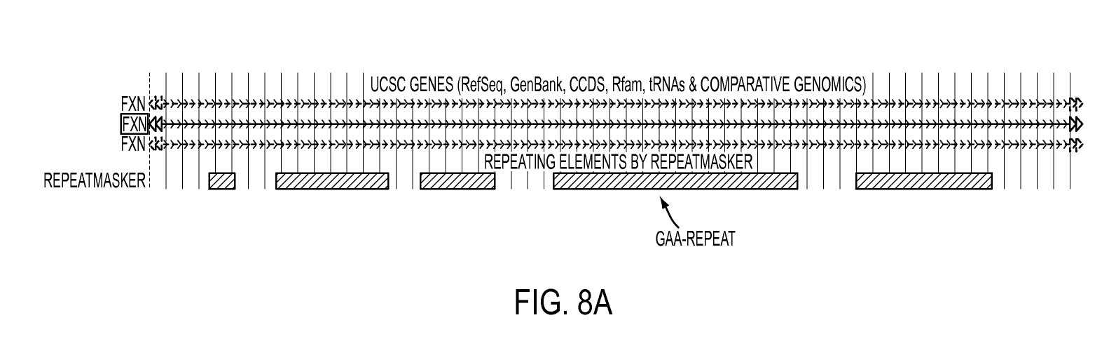

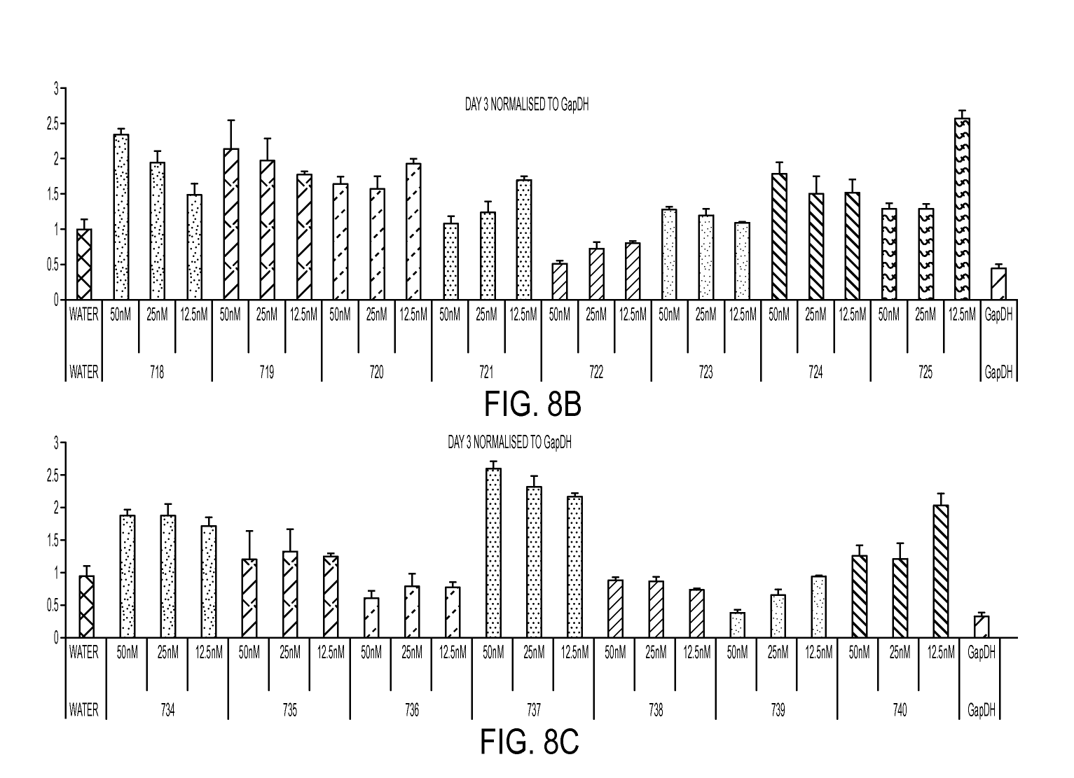

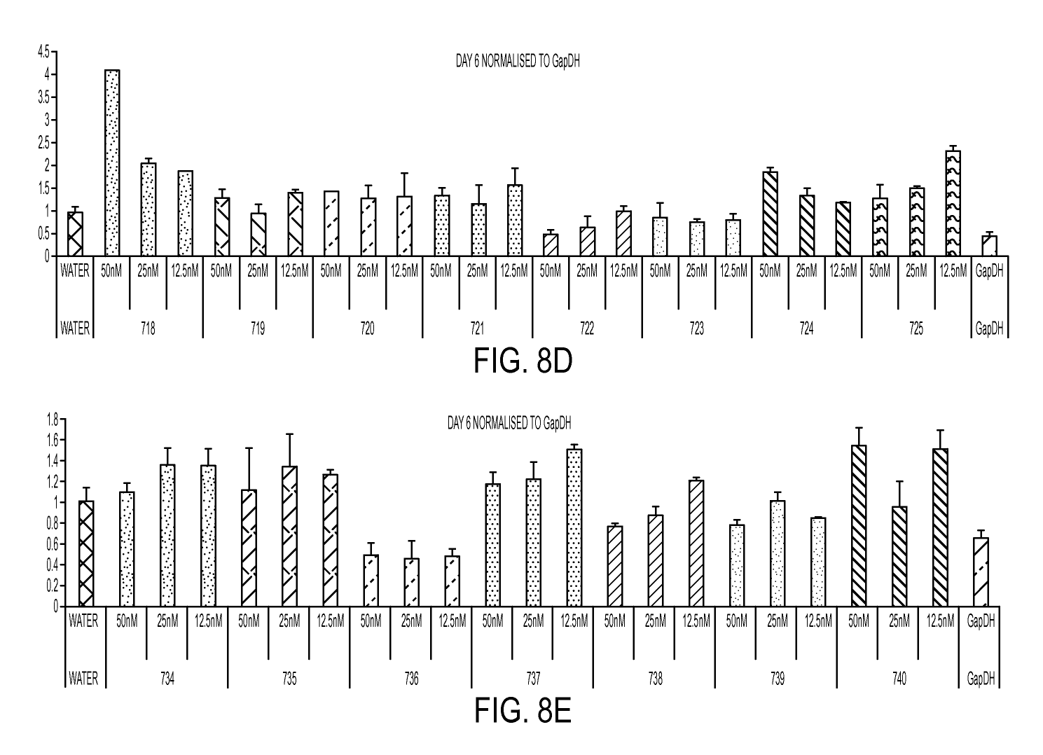

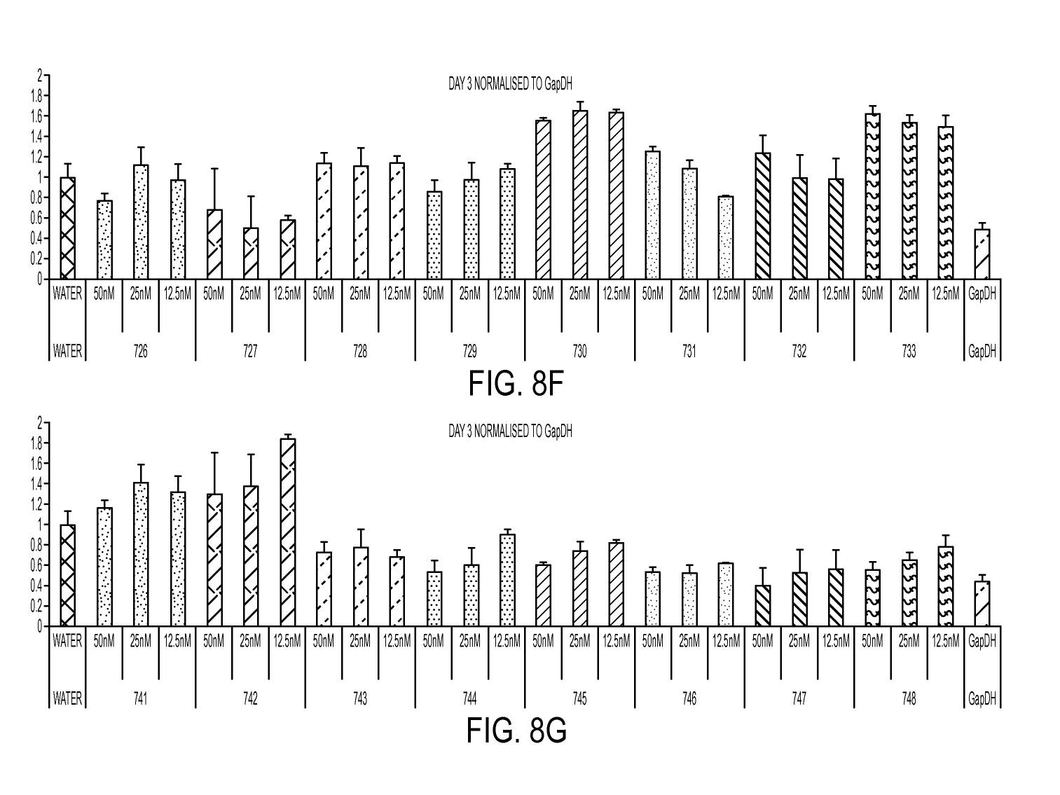

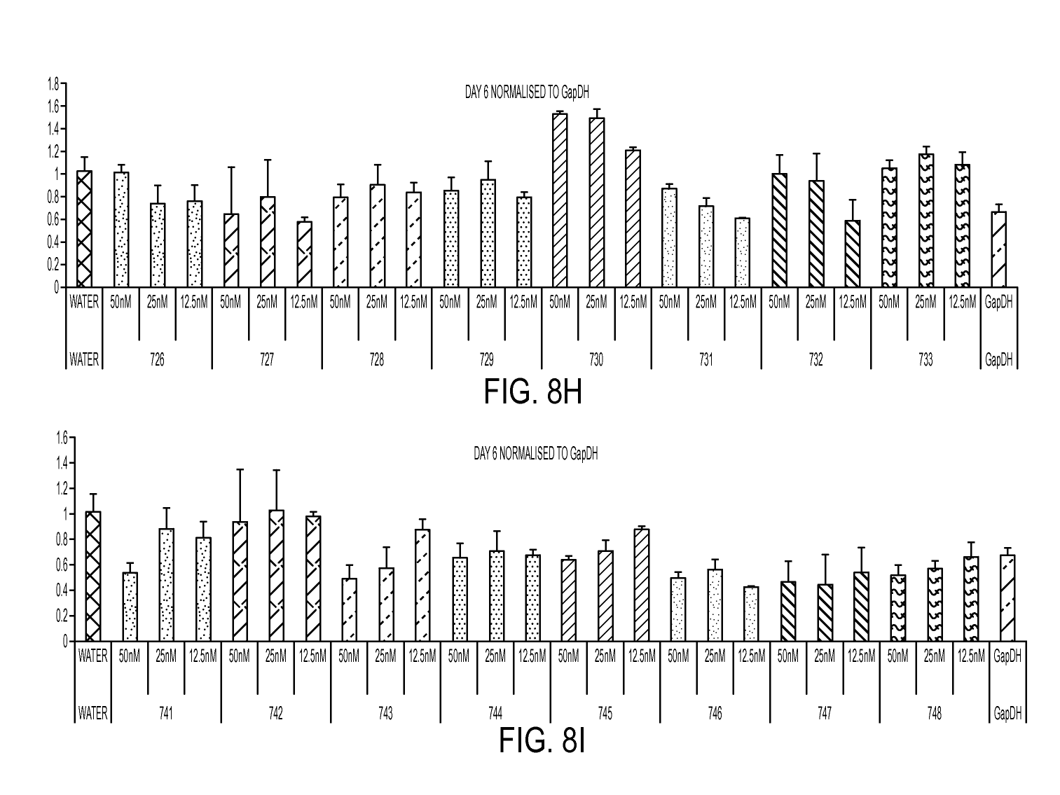

Further gapmer and mixmer oligonucleotides were designed to target the repeat regions present in the first intron of the FXN gene or the nucleic acid regions flanking the repeat regions present in the first intron of the FXN gene (FIG. 8A shows the location of the repeat region). The sequence and structure of each gapmer and mixmer oligonucleotide is shown in Table 5. Table 4 provides a description of the nucleotide analogs, modifications and intranucleotide linkages used for certain oligonucleotides tested and described in Table 5.

TABLE 5
Further gapmer and mixmer oligonucleotides

| SEQ | | | | | |
|---|---|---|---|---|---|
| ID | | | | | |
| NO | Oligo name | Base sequence | Gene | Species | Formatted Sequence |
| 3 | FXN-718 m08 | GGGATCCCTTCAGAG | FXN | Human | lnaGs; lnaGs; |
| | | | | | lnaGs; dAs; |
| | | | | | dTs; dCs; |
| | | | | | dCs; dCs; |
| | | | | | dTs; dTs; |
| | | | | | dCs; dAs; |
| | | | | | lnaGs; lnaAs; |
| | | | | | lnaG-Sup |
| 4 | FXN-719 m08 | TGGCTGGTACGCCGC | FXN | Human | lnaTs; lnaGs; |
| | | | | | lnaGs; dCs; |
| | | | | | dTs; dGs; |
| | | | | | dGs; dTs; |
| | | | | | dAs; dCs; |
| | | | | | dGs; dCs; |
| | | | | | lnaCs; lnaGs; |
| | | | | | lnaC-Sup |
| 5 | FXN-720 m08 | ACGCCGCATGTATTA | FXN | Human | lnaAs; lnaCs; |
| | | | | | lnaGs; dCs; |
| | | | | | dCs; dGs; |
| | | | | | dCs; dAs; |
| | | | | | dTs; dGs; |
| | | | | | dTs; dAs; |
| | | | | | lnaTs; lnaTs; |
| | | | | | lnaA-Sup |
| 6 | FXN-721 m08 | AGATGAAAGAGGCAG | FXN | Human | lnaAs; lnaGs; |
| | | | | | lnaAs; dTs; |
| | | | | | dGs; dAs; |
| | | | | | dAs; dAs; |
| | | | | | dGs; dAs; |
| | | | | | dGs; dGs; |
| | | | | | lnaCs; lnaAs; |
| | | | | | lnaG-Sup |
| 7 | FXN-722 m08 | GCCACGTCCAAGCCA | FXN | Human | lnaGs; lnaCs; |
| | | | | | lnaCs; dAs; |
| | | | | | dCs; dGs; |
| | | | | | dTs; dCs; |
| | | | | | dCs; dAs; |
| | | | | | dAs; dGs; |
| | | | | | lnaCs; lnaCs; |
| | | | | | lnaA-Sup |
| 8 | FXN-723 m08 | TATTTGTGTTGCTCT | FXN | Human | lnaTs; lnaAs; |
| | | | | | lnaTs; dTs; |
| | | | | | dTs; dGs; |
| | | | | | dTs; dGs; |
| | | | | | dTs; dTs; |
| | | | | | dGs; dCs; |
| | | | | | lnaTs; lnaCs; |
| | | | | | lnaT-Sup |
| 9 | FXN-724 m08 | CCGGAGTTTGTACTT | FXN | Human | lnaCs; lnaCs; |
| | | | | | lnaGs; dGs; |
| | | | | | dAs; dGs; |
| | | | | | dTs; dTs; |
| | | | | | dTs; dGs; |
| | | | | | dTs; dAs; |
| | | | | | lnaCs; lnaTs; |
| | | | | | lnaT-Sup |
| 10 | FXN-725 m08 | TAGGCTTGAACTTCC | FXN | Human | lnaTs; lnaAs; |
| | | | | | lnaGs; dGs; |
| | | | | | dCs; dTs; |
| | | | | | dTs; dGs; |
| | | | | | dAs; dAs; |
| | | | | | dCs; dTs; |
| | | | | | lnaTs; lnaCs; |
| | | | | | lnaC-Sup |
| 11 | FXN-726 m08 | CACACGTGTTATTTG | FXN | Human | lnaCs; lnaAs; |
| | | | | | lnaCs; dAs; |
| | | | | | dCs; dGs; |
| | | | | | dTs; dGs; |
| | | | | | dTs; dTs; |
| | | | | | dAs; dTs; |
| | | | | | lnaTs; lnaTs; |
| | | | | | lnaG-Sup |
| 12 | FXN-727 m08 | GCCCACATTGTGTTT | FXN | Human | lnaGs; lnaCs; |
| | | | | | lnaCs; dCs; |
| | | | | | dAs; dCs; |
| | | | | | dAs; dTs; |
| | | | | | dTs; dGs; |
| | | | | | dTs; dGs; |
| | | | | | lnaTs; lnaTs; |
| | | | | | lnaT-Sup |
| 13 | FXN-728 m08 | GAAGAAACTTTGGGA | FXN | Human | lnaGs; lnaAs; |
| | | | | | lnaAs; dGs; |
| | | | | | dAs; dAs; |
| | | | | | dAs; dCs; |
| | | | | | dTs; dTs; |
| | | | | | dTs; dGs; |
| | | | | | lnaGs; lnaGs; |
| | | | | | lnaA-Sup |
| 14 | FXN-729 m08 | TTGGTTGCCAGTGCT | FXN | Human | lnaTs; lnaTs; |
| | | | | | lnaGs; dGs; |
| | | | | | dTs; dTs; |
| | | | | | dGs; dCs; |
| | | | | | dCs; dAs; |
| | | | | | dGs; dTs; |
| | | | | | lnaGs; lnaCs; |
| | | | | | lnaT-Sup |
| 15 | FXN-730 m08 | TAAAAGTTAGGACTT | FXN | Human | lnaTs; lnaAs; |
| | | | | | lnaAs; dAs; |
| | | | | | dAs; dGs; |
| | | | | | dTs; dTs; |
| | | | | | dAs; dGs; |
| | | | | | dGs; dAs; |
| | | | | | lnaCs; lnaTs; |
| | | | | | lnaT-Sup |
| 16 | FXN-731 m08 | AGAAAATGGATTTCC | FXN | Human | lnaAs; lnaGs; |
| | | | | | lnaAs; dAs; |
| | | | | | dAs; dAs; |
| | | | | | dTs; dGs; |
| | | | | | dGs; dAs; |
| | | | | | dTs; dTs; |
| | | | | | lnaTs; lnaCs; |
| | | | | | lnaC-Sup |
| 17 | FXN-732 m08 | TGGCAGGACGCGGTG | FXN | Human | lnaTs; lnaGs; |
| | | | | | lnaGs; dCs; |
| | | | | | dAs; dGs; |
| | | | | | dGs; dAs; |
| | | | | | dCs; dGs; |
| | | | | | dCs; dGs; |
| | | | | | lnaGs; lnaTs; |
| | | | | | lnaG-Sup |
| 18 | FXN-733 m08 | TTAGATCTCCTCTAG | FXN | Human | lnaTs; lnaTs; |
| | | | | | lnaAs; dGs; |
| | | | | | dAs; dTs; |
| | | | | | dCs; dTs; |
| | | | | | dCs; dCs; |
| | | | | | dTs; dCs; |
| | | | | | lnaTs; lnaAs; |
| | | | | | lnaG-Sup |
| 19 | FXN-734 m08 | GAAAGCAGACATTTA | FXN | Human | lnaGs; lnaAs; |
| | | | | | lnaAs; dAs; |
| | | | | | dGs; dCs; |
| | | | | | dAs; dGs; |
| | | | | | dAs; dCs; |
| | | | | | dAs; dTs; |
| | | | | | lnaTs; lnaTs; |
| | | | | | lnaA-Sup |
| 20 | FXN-735 m08 | TTACTTGGCTTCTGT | FXN | Human | lnaTs; lnaTs; |
| | | | | | lnaAs; dCs; |
| | | | | | dTs; dTs; |
| | | | | | dGs; dGs; |
| | | | | | dCs; dTs; |
| | | | | | dTs; dCs; |
| | | | | | lnaTs; lnaGs; |
| | | | | | lnaT-Sup |
| 21 | FXN-736 m08 | CACTATCTGAGCTGC | FXN | Human | lnaCs; lnaAs; |
| | | | | | lnaCs; dTs; |
| | | | | | dAs; dTs; |
| | | | | | dCs; dTs; |
| | | | | | dGs; dAs; |
| | | | | | dGs; dCs; |
| | | | | | lnaTs; lnaGs; |
| | | | | | lnaC-Sup |
| 22 | FXN-737 m08 | CACGTATTGGGCTTC | FXN | Human | lnaCs; lnaAs; |
| | | | | | lnaCs; dGs; |
| | | | | | dTs; dAs; |
| | | | | | dTs; dTs; |
| | | | | | dGs; dGs; |
| | | | | | dGs; dCs; |
| | | | | | lnaTs; lnaTs; |
| | | | | | lnaC-Sup |
| 23 | FXN-738 m08 | CACCCCTGCCTGTGT | FXN | Human | lnaCs; lnaAs; |
| | | | | | lnaCs; dCs; |
| | | | | | dCs; dCs; |
| | | | | | dTs; dGs; |
| | | | | | dCs; dCs; |
| | | | | | dTs; dGs; |
| | | | | | lnaTs; lnaGs; |
| | | | | | lnaT-Sup |
| 24 | FXN-739 m08 | GGACAGCATGGGTTG | FXN | Human | lnaGs; lnaGs; |
| | | | | | lnaAs; dCs; |
| | | | | | dAs; dGs; |
| | | | | | dCs; dAs; |
| | | | | | dTs; dGs; |
| | | | | | dGs; dGs; |
| | | | | | lnaTs; lnaTs; |
| | | | | | lnaG-Sup |
| 25 | FXN-740 m08 | GTCAGCAGAGTTGTG | FXN | Human | lnaGs; lnaTs; |
| | | | | | lnaCs; dAs; |
| | | | | | dGs; dCs; |
| | | | | | dAs; dGs; |
| | | | | | dAs; dGs; |
| | | | | | dTs; dTs; |
| | | | | | lnaGs; lnaTs; |
| | | | | | lnaG-Sup |
| 26 | FXN-741 m08 | TGGATTTCCCAGCAT | FXN | Human | lnaTs; lnaGs; |
| | | | | | lnaGs; dAs; |
| | | | | | dTs; dTs; |
| | | | | | dTs; dCs; |
| | | | | | dCs; dCs; |
| | | | | | dAs; dGs; |
| | | | | | lnaCs; lnaAs; |
| | | | | | lnaT-Sup |
| 27 | FXN-742 m08 | TAGGCAAGTGTGGCC | FXN | Human | lnaTs; lnaAs; |
| | | | | | lnaGs; dGs; |
| | | | | | dCs; dAs; |
| | | | | | dAs; dGs; |
| | | | | | dTs; dGs; |
| | | | | | dTs; dGs; |
| | | | | | lnaGs; lnaCs; |
| | | | | | lnaC-Sup |
| 28 | FXN-743 m08 | TGGCCATGATGGTCC | FXN | Human | lnaTs; lnaGs; |
| | | | | | lnaGs; dCs; |
| | | | | | dCs; dAs; |
| | | | | | dTs; dGs; |
| | | | | | dAs; dTs; |
| | | | | | dGs; dGs; |
| | | | | | lnaTs; lnaCs; |
| | | | | | lnaC-Sup |
| 29 | FXN-744 m08 | CCGGAGTTCAAGACT | FXN | Human | lnaCs; lnaCs; |
| | | | | | lnaGs; dGs; |
| | | | | | dAs; dGs; |
| | | | | | dTs; dTs; |
| | | | | | dCs; dAs; |
| | | | | | dAs; dGs; |
| | | | | | lnaAs; lnaCs; |
| | | | | | lnaT-Sup |
| 30 | FXN-745 m08 | AACCCAGTATCTACT | FXN | Human | lnaAs; lnaAs; |
| | | | | | lnaCs; dCs; |
| | | | | | dCs; dAs; |
| | | | | | dGs; dTs; |
| | | | | | dAs; dTs; |
| | | | | | dCs; dTs; |
| | | | | | lnaAs; lnaCs; |
| | | | | | lnaT-Sup |
| 31 | FXN-746 m08 | GTTAGCCGGGCGTGG | FXN | Human | lnaGs; lnaTs; |
| | | | | | lnaTs; dAs; |
| | | | | | dGs; dCs; |
| | | | | | dCs; dGs; |
| | | | | | dGs; dGs; |
| | | | | | dCs; dGs; |
| | | | | | lnaTs; lnaGs; |
| | | | | | lnaG-Sup |
| 32 | FXN-747 m08 | TGTAATCCCAGCTAC | FXN | Human | lnaTs; lnaGs; |
| | | | | | lnaTs; dAs; |
| | | | | | dAs; dTs; |
| | | | | | dCs; dCs; |
| | | | | | dCs; dAs; |
| | | | | | dGs; dCs; |
| | | | | | lnaTs; lnaAs; |
| | | | | | lnaC-Sup |
| 33 | FXN-748 m08 | TCCAGAGGCTGCGGC | FXN | Human | lnaTs; lnaCs; |
| | | | | | lnaCs; dAs; |
| | | | | | dGs; dAs; |
| | | | | | dGs; dGs; |
| | | | | | dCs; dTs; |
| | | | | | dGs; dCs; |
| | | | | | lnaGs; lnaGs; |
| | | | | | lnaC-Sup |
| 34 | FXN-115 m01 | GAAGAAGAAGAAGAA | FXN | human | lnaGs; omeAs; |
| | | | | | lnaAs; omeGs; |
| | | | | | lnaAs; omeAs; |
| | | | | | lnaGs; omeAs; |
| | | | | | lnaAs; omeGs; |
| | | | | | lnaAs; omeAs; |
| | | | | | lnaGs; omeAs; |
| | | | | | lnaA-Sup |
| 35 | FXN-116 m12 | GAAGAAGAAGAAGAA | FXN | human | lnaGs; dAs; |
| | | | | | lnaAs; dGs; |
| | | | | | lnaAs; dAs; |
| | | | | | lnaGs; dAs; |
| | | | | | lnaAs; dGs; |
| | | | | | lnaAs; dAs; |
| | | | | | lnaGs; dAs; |
| | | | | | lnaA-Sup |
| 36 | FXN-117 m01 | TTCTTCTTCTTCTTC | FXN | human | lnaTs; omeUs; |
| | | | | | lnaCs; omeUs; |
| | | | | | lnaTs; omeCs; |
| | | | | | lnaTs; omeUs; |
| | | | | | lnaCs; omeUs; |
| | | | | | lnaTs; omeCs; |
| | | | | | lnaTs; omeUs; |
| | | | | | lnaC-Sup |
| 37 | FXN-117 m12 | TTCTTCTTCTTCTTC | FXN | human | lnaTs; dTs; |
| | | | | | lnaCs; dTs; |
| | | | | | lnaTs; dCs; |
| | | | | | lnaTs; dTs; |
| | | | | | lnaCs; dTs; |
| | | | | | lnaTs; dCs; |
| | | | | | lnaTs; dTs; |
| | | | | | lnaC-Sup |
| 38 | FXN-119 m01 | CTTCTTCTTCTTCTT | FXN | human | lnaCs; omeUs; |
| | | | | | lnaTs; omeCs; |
| | | | | | lnaTs; omeUs; |
| | | | | | lnaCs; omeUs; |
| | | | | | lnaTs; omeCs; |
| | | | | | lnaTs; omeUs; |
| | | | | | lnaCs; omeUs; |
| | | | | | lnaT-Sup |
| 39 | FXN-119 m09 | CTTCTTCTTCTTCTT | FXN | human | lnaCs; dTs; |
| | | | | | lnaTs; dCs; |
| | | | | | lnaTs; dTs; |
| | | | | | lnaCs; dTs; |
| | | | | | lnaTs; dCs; |
| | | | | | lnaTs; dTs; |
| | | | | | lnaCs; dTs; |
| | | | | | lnaT-Sup |
| 40 | FXN-121 m09 | GAAGAAGA | FXN | human | lnaGs; lnaAs; |
| | | | | | lnaAs; lnaGs; |
| | | | | | lnaAs; lnaAs; |
| | | | | | lnaGs; lnaA-Sup |
| 41 | FXN-122 m09 | AAGAAGAA | FXN | human | lnaAs; lnaAs; |
| | | | | | lnaGs; lnaAs; |
| | | | | | lnaAs; lnaGs; |
| | | | | | lnaAs; lnaA-Sup |
| 42 | FXN-123 m09 | AGAAGAAG | FXN | human | lnaAs; lnaGs; |
| | | | | | lnaAs; lnaAs; |
| | | | | | lnaGs; lnaAs; |
| | | | | | lnaAs; lnaG-Sup |
| 43 | FXN-124 m09 | TTCTTCTT | FXN | human | lnaTs; lnaTs; |
| | | | | | lnaCs; lnaTs; |
| | | | | | lnaTs; lnaCs; |
| | | | | | lnaTs; lnaT-Sup |
| 44 | FXN-125 m09 | CTTCTTCT | FXN | human | lnaCs; lnaTs; |
| | | | | | lnaTs; lnaCs; |
| | | | | | lnaTs; lnaTs; |
| | | | | | lnaCs; lnaT-Sup |
| 45 | FXN-320 m01 | AAGAAGAAGAAGAAG | FXN | human | lnaAs; omeAs; |
| | | | | | lnaGs; omeAs; |
| | | | | | lnaAs; omeGs; |
| | | | | | lnaAs; omeAs; |
| | | | | | lnaGs; omeAs; |
| | | | | | lnaAs; omeGs; |
| | | | | | lnaAs; omeAs; |
| | | | | | lnaG-Sup |
| 46 | FXN-321 m01 | AGAAGAAGAAGAAGA | FXN | human | lnaAs; omeGs; |
| | | | | | lnaAs; omeAs; |
| | | | | | lnaGs; omeAs; |
| | | | | | lnaAs; omeGs; |
| | | | | | lnaAs; omeAs; |
| | | | | | lnaGs; omeAs; |
| | | | | | lnaAs; omeGs; |
| | | | | | lnaA-Sup |
| 47 | FXN-322 m01 | TCTTCTTCTTCTTCT | FXN | human | lnaTs; omeCs; |
| | | | | | lnaTs; omeUs; |
| | | | | | lnaCs; omeUs; |
| | | | | | lnaTs; omeCs; |
| | | | | | lnaTs; omeUs; |
| | | | | | lnaCs; omeUs; |
| | | | | | lnaTs; omeCs; |
| | | | | | lnaT-Sup |
| 48 | FXN-115 m08 | GAAGAAGAAGAAGAA | FXN | human | lnaGs; lnaAs; |
| | | | | | lnaAs; dGs; |
| | | | | | dAs; dAs; |
| | | | | | dGs; dAs; |
| | | | | | dAs; dGs; |
| | | | | | dAs; dAs; |
| | | | | | lnaGs; lnaAs; |
| | | | | | lnaA-Sup |
| 49 | FXN-117 m08 | TTCTTCTTCTTCTTC | FXN | human | lnaTs; lnaTs; |
| | | | | | lnaCs; dTs; |
| | | | | | dTs; dCs; |
| | | | | | dTs; dTs; |
| | | | | | dCs; dTs; |
| | | | | | dTs; dCs; |
| | | | | | lnaTs; lnaTs; |
| | | | | | lnaC-Sup |
| 50 | FXN-121 m12 | GAAGAAGA | FXN | human | lnaGs; dAs; |
| | | | | | lnaAs; dGs; |
| | | | | | lnaAs; dAs; |
| | | | | | lnaGs; dA-Sup |
| 51 | FXN-122 m12 | AAGAAGAA | FXN | human | lnaAs; dAs; |
| | | | | | lnaGs; dAs; |
| | | | | | lnaAs; dGs; |
| | | | | | lnaAs; dA-Sup |
| 52 | FXN-123 m12 | AGAAGAAG | FXN | human | lnaAs; dGs; |
| | | | | | lnaAs; dAs; |
| | | | | | lnaGs; dAs; |
| | | | | | lnaAs; dG-Sup |
| 53 | FXN-124 m12 | TTCTTCTT | FXN | human | lnaTs; dTs; |
| | | | | | lnaCs; dTs; |
| | | | | | lnaTs; dCs; |
| | | | | | lnaTs; dT-Sup |
| 54 | FXN-125 m12 | CTTCTTCT | FXN | human | lnaCs; dTs; |
| | | | | | lnaTs; dCs; |
| | | | | | lnaTs; dTs; |
| | | | | | lnaCs; dT-Sup |
| 55 | FXN-323 m12 | TCTTCTTC | FXN | human | lnaTs; dCs; |
| | | | | | lnaTs; dTs; |
| | | | | | lnaCs; dTs; |
| | | | | | lnaTs; dC-Sup |
| 56 | FXN-115 m08 | GAAGAAGAAGAAGAA | FXN | human | lnaGs; lnaAs; |
| | | | | | lnaAs; dGs; |
| | | | | | dAs; dAs; |
| | | | | | dGs; dAs; |
| | | | | | dAs; dGs; |
| | | | | | dAs; dAs; |
| | | | | | lnaGs; lnaAs; |
| | | | | | lnaA-Sup |
| 57 | FXN-117 m08 | TTCTTCTTCTTCTTC | FXN | human | lnaTs; lnaTs; |
| | | | | | lnaCs; dTs; |
| | | | | | dTs; dCs; |
| | | | | | dTs; dTs; |
| | | | | | dCs; dTs; |
| | | | | | dTs; dCs; |
| | | | | | lnaTs; lnaTs; |
| | | | | | lnaC-Sup |
| 58 | FXN-320 m08 | AAGAAGAAGAAGAAG | FXN | human | lnaAs; lnaAs; |
| | | | | | lnaGs; dAs; |
| | | | | | dAs; dGs; |
| | | | | | dAs; dAs; |
| | | | | | dGs; dAs; |
| | | | | | dAs; dGs; |
| | | | | | lnaAs; lnaAs; |
| | | | | | lnaG-Sup |
| 59 | FXN-321 m08 | AGAAGAAGAAGAAGA | FXN | human | lnaAs; lnaGs; |
| | | | | | lnaAs; dAs; |
| | | | | | dGs; dAs; |
| | | | | | dAs; dGs; |
| | | | | | dAs; dAs; |
| | | | | | dGs; dAs; |
| | | | | | lnaAs; lnaGs; |
| | | | | | lnaA-Sup |
| 60 | FXN-322 m08 | TCTTCTTCTTCTTCT | FXN | human | lnaTs; lnaCs; |
| | | | | | lnaTs; dTs; |
| | | | | | dCs; dTs; |
| | | | | | dTs; dCs; |
| | | | | | dTs; dTs; |
| | | | | | dCs; dTs; |
| | | | | | lnaTs; lnaCs; |
| | | | | | lnaT-Sup |
| 61 | FXN-119 m08 | CTTCTTCTTCTTCTT | FXN | human | lnaCs; lnaTs; |
| | | | | | lnaTs; dCs; |
| | | | | | dTs; dTs; |
| | | | | | dCs; dTs; |
| | | | | | dTs; dCs; |
| | | | | | dTs; dTs; |
| | | | | | lnaCs; lnaTs; |
| | | | | | lnaT-Sup |
| 62 | FXN-115 m08 | GAAGAAGAAGAAGAA | FXN | human | lnaGs; lnaAs; |
| | | | | | lnaAs; dGs; |
| | | | | | dAs; dAs; |
| | | | | | dGs; dAs; |
| | | | | | dAs; dGs; |
| | | | | | dAs; dAs; |
| | | | | | lnaGs; lnaAs; |
| | | | | | lnaA-Sup |

31 oligos from Table 5 were screened in GM03816 fibroblast cell lines by transfection at three concentrations (50 nM, 25 nM, 12.5 nM). Collections were done at day 3 and day 6 post transfection. FIG. 8B-I show FXN mRNA upregulation at day 3 and day 6 following treatment with the various oligos. Oligos FXN-718 and 724 gave dose dependent FXN mRNA upregulation at day 3 and day 6. Oligos FXN-719, 730, 734 and 737 gave dose-dependent FXN mRNA upregulation at day 3 and/or at day 6.

### Example 4

Argonaute (Ago) recruitment to the FXN gene locus was examined in FRDA diseased (GM15850, GM16209) cells relative to normal (GM15851) cells. Ago is a component of the RNA-induced silencing complex (RISC). Without wishing to be bound by theory, RNAs guide Ago to nucleic acid regions through sequence complementarity, which typically leads to silencing of the target.

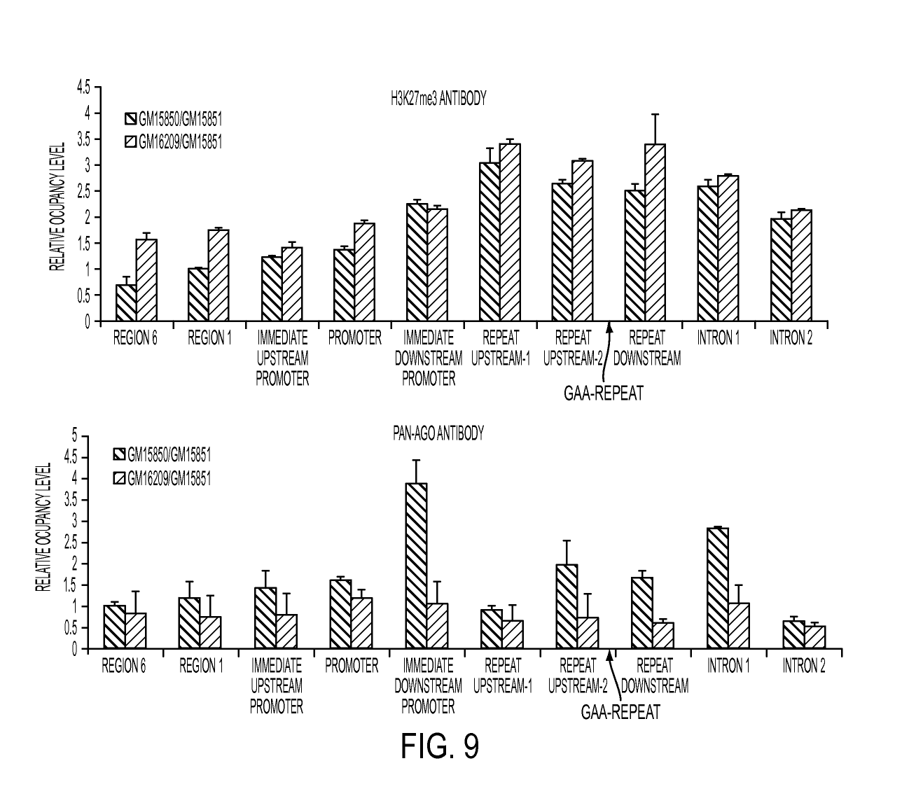

H3K27me3 and Pan-Ago chromatin immunoprecipitations (ChIP) were done side-by-side. The antibodies used were H3K27me3 (Abcam ab6002) and pan-Ago (Millipore 03-248). ChIP with the H3K27me3 antibody showed the expected pattern of H3K27me3 localization around the repeat region of FXN (FIG. 9). Ago enrichment level was found to be potentially higher around heterochromatin border regions of FXN than within the heterochromatic region in GM15850 cells (FIG. 9). This finding supports Ago involvement in FXN epigenetic state in diseased cells.

Without further elaboration, it is believed that one skilled in the art can, based on the description provided herein, utilize the present invention to its fullest extent. The specific embodiments are, therefore, to be construed as merely illustrative, and not limitative of the remainder of the disclosure in any way whatsoever. All publications cited herein are incorporated by reference for the purposes or subject matter referenced herein.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1.-53. (canceled)

54. A method for increasing expression of a target gene in cells, the method comprising:
delivering to the cells an oligonucleotide complementary to a heterochromatin forming non-coding RNA associated with the target gene, wherein the oligonucleotide comprises a region of complementarity that is complementary with a position adjacent to a repeat region of the heterochromatin forming non-coding RNA.

55. The method of claim 54, wherein the oligonucleotide further comprises a region of complementarity that is complementary with a position within the repeat region of the heterochromatin forming non-coding RNA.

56. The method of claim 54, wherein the oligonucleotide is a cleavage promoting oligonucleotide.

57. The method of claim 54, wherein the RNA is a long non-coding RNA (lncRNA).

58. The method of claim 57, wherein the lncRNA is antisense to the gene.

59. The method of claim 54, wherein the repeat region comprises triplet repeats.

60. The method of claim 54, wherein the oligonucleotide comprises a region of complementarity that is complementary with a position within 5 kb from an end of the repeat region.

61. The method of claim 54, wherein the oligonucleotide is single stranded.

62. The method of claim 61, wherein the oligonucleotide comprises at least one modified nucleotide.

63. The method of claim 62, wherein the modified nucleotide is a bridged nucleotide.

64. The method of claim 63, wherein the bridge nucleotide is a locked nucleic acid (LNA) nucleotide, a constrained ethyl (cEt) nucleotide, or an ethylene bridged nucleic acid (ENA) nucleotide.

65. The method of claim 62, wherein the modified nucleotide is 2′ O-methyl nucleotide.

66. The method of claim 56, wherein the oligonucleotide is a gapmer.
